(19) United States
(12) Patent Application Publication
(10) Pub. No.: US 2003/0206887 A1
(43) Pub. Date: Nov. 6, 2003

(54) RNA interference mediated inhibition of hepatitis B virus (HBV) using short interfering nucleic acid (siNA)

(76) Inventors: David Morrissey, Boulder, CO (US); James A. McSwiggen, Boulder, CO (US); Leonid Beigelman, Longmont, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/244,647

(22) Filed: Sep. 16, 2002

Related U.S. Application Data
(63) Continuation-in-part of application No. PCT/US02/09187, filed on Mar. 26, 2002.
(63) Continuation-in-part of application No. 09/877,478, filed on Jun. 8, 2001, now abandoned.
Application No. 09/877,478 is a continuation-in-part of application No. 09/696,347, filed on Oct. 24, 2000, now abandoned.
Application No. 09/696,347 is a continuation-in-part of application No. 09/636,385, filed on Aug. 9, 2000, now abandoned.
Application No. 09/636,385 is a continuation-in-part of application No. 09/531,025, filed on Mar. 20, 2000, now abandoned.
Application No. 09/531,025 is a continuation-in-part of application No. 09/436,430, filed on Nov. 8, 1999.
Application No. 09/436,430 is a continuation of application No. 08/193,627, filed on Feb. 7, 1994, now Pat. No. 6,017,756.
Application No. 08/193,627 is a continuation of application No. 07/882,712, filed on May 14, 1992, now abandoned.
(60) Provisional application No. 60/296,876, filed on Jun. 8, 2001.
(60) Provisional application No. 60/335,059, filed on Oct. 24, 2001.
(60) Provisional application No. 60/337,055, filed on Dec. 5, 2001.
(60) Provisional application No. 60/358,580, filed on Feb. 20, 2002.
(60) Provisional application No. 60/363,124, filed on Mar. 11, 2002.
(60) Provisional application No. 60/386,782, filed on Jun. 6, 2002.
(60) Provisional application No. 60/406,784, filed on Aug. 29, 2002.
(60) Provisional application No. 60/408,378, filed on Sep. 5, 2002.
(60) Provisional application No. 60/409,293, filed on Sep. 9, 2002.

(51) Int. Cl. C07H 21/02; A61K 48/00

(52) U.S. Cl. 424/93.2; 514/44; 536/23.1

(57) ABSTRACT

The present invention concerns methods and reagents useful in modulating hepatitis B virus (HBV) gene expression in a variety of applications, including use in therapeutic, diagnostic, target validation, and genomic discovery applications. Specifically, the invention relates to short interfering nucleic acid (siNA) or short interfering RNA (siRNA) molecules capable of mediating RNA interference (RNAi) against against hepatitis B virus (HBV).

### PRIORITY

This application claims the benefit of U.S. application Ser. Nos. 60/358,580, filed Feb. 20, 2002, and 60/393,924, filed Jul. 3, 2002. This application also claims priority to PCT US02/09187, filed Mar. 26, 2002, which claims the benefit of U.S. application Ser. No. 60/296,876, filed Jun. 8, 2001.

### BACKGROUND OF THE INVENTION

The present invention concerns methods and reagents useful in modulating hepatitis B virus (HBV) gene expression and activity in a variety of applications, including use in therapeutic, diagnostic, target validation, and genomic discovery applications. Specifically, the invention relates to short interfering nucleic acid (siNA) molecules capable of mediating RNA interference (RNAi) against HBV expression.

The following is a discussion of relevant art pertaining to RNAi. The discussion is provided only for understanding of the invention that follows. The summary is not an admission that any of the work described below is prior art to the claimed invention.

RNA interference refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., 2001, Nature, 409, 363). Short interfering RNAs derived from dicer activity are typically about 21-23 nucleotides in length and comprise about 19 base pair duplexes. Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal di-nucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the siRNA guide sequence (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two 2-nucleotide 3′-overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836, both suggest that siRNA “may include modifications to either the phosphate-sugar backbone or the nucleoside. . . to include at least one of a nitrogen or sulfur heteroatom”; however, neither application teaches to what extent these modifications are tolerated in siRNA molecules nor provides any examples of such modified siRNA. Kreutzer and Limmer, Canadian Patent Application No. 2,359,180, also describe certain chemical modifications for use in dsRNA constructs in order to counteract activation of double-stranded-RNA-dependent protein kinase PKR, specifically 2′-amino or 2′-O-methyl nucleotides, and nucleotides containing a 2′-O or 4′-C methylene bridge. However, Kreutzer and Limmer similarly fail to show to what extent these modifications are tolerated in siRNA molecules nor do they provide any examples of such modified siRNA.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that “RNAs with two [phosphorothioate]-modified bases also had substantial decreases in effectiveness as RNAi triggers (data not shown); [phosphorothioate] modification of more than two residues greatly destabilized the RNAs in vitro and we were not able to assay interference activities.” Id. at 1081. The authors also tested certain modifications at the 2′-position of the nucleotide sugar in the long siRNA transcripts and observed that substituting deoxynucleotides for ribonucleotides “produced a substantial decrease in interference activity,” especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. They found that whereas 4-thiouracil and 5-bromouracil were all well tolerated, inosine “produced a substantial decrease in interference activity” when incorporated in either strand. Incorporation of 5-iodouracil and 3-(aminoallyl)uracil in the antisense strand resulted in substantial decrease in RNAi activity as well.

Beach et al., International PCT Publication No. WO 01/68836, describe specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due “to the danger of activating interferon response.” Li et al., International PCT Publication No. WO 00/44914, describe the use of specific dsRNAs for use in attenuating the expression of certain target genes. Zernicka-Goetz et al., International PCT Publication No. WO 01/36646, describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA-mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells. Driscoll et al., International PCT Publication No. WO 01/49844, describe specific DNA constructs for use in facilitating gene silencing in targeted organisms. Parrish et al., 2000, Molecular Cell, 6, 1977-1087, describe specific chemically-modified siRNA constructs targeting the unc-22 gene of C. elegans. Grossniklaus, International PCT Publication No. WO 01/38551, describes certain methods for regulating polycomb gene expression in plants. Churikov et al., International PCT Publication No. WO 01/42443, describes certain methods for modifying genetic characteristics of an organism. Cogoni et al., International PCT Publication No. WO 01/53475, describes certain methods for isolating a Neurospora silencing gene and uses thereof. Reed et al., International PCT Publication No. WO 01/68836, describes certain methods for gene silencing in plants. Honer et al., International PCT Publication No. WO 01/70944, describe certain methods of drug screening using transgenic nematodes as Parkinson's Disease models. Deak et al., International PCT Publication No. WO 01/72774, describe certain Drosophila-derived gene products. Arndt et al., International PCT Publication No. WO 01/92513, describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences. Echeverri et al., International PCT Publication No. WO 02/38805, describe certain C. elegans genes identified via RNAi. Kreutzer et al., International PCT Publications Nos. WO 02/055692 and WO 02/055693, describe certain methods for inhibiting gene expression using RNAi.

Chronic hepatitis B is caused by an enveloped virus, commonly known as the hepatitis B virus or HBV. HBV is transmitted via infected blood or other body fluids, especially saliva and semen, during delivery of a child, sexual activity, or sharing of needles contaminated by infected blood. Individuals can be “carriers” and transmit the infection to others without ever having experienced symptoms of the disease. Persons at highest risk are those with multiple sex partners, those with a history of sexually transmitted diseases, parenteral drug users, infants born to infected mothers, “close” contacts of or sexual partners of infected persons, and healthcare personnel or other service employees who have contact with blood. Transmission is also possible via tattooing, ear or body piercing, and acupuncture; the virus is also stable on razors, toothbrushes, baby bottles, eating utensils, and some hospital equipment such as respirators, scopes, and instruments. There is no evidence that HbsAg (an HBV surface antigen)-positive food handlers pose a health risk in an occupational setting; hence, they should not be excluded from the workplace. Hepatitis B has never been documented as being a food-borne disease. The average incubation period is 60 to 90 days, with a range of 45 to 180 days; the number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of severity include: (1) the size of the dose to which the person was exposed; (2) the person's age, with younger patients experiencing a milder form of the disease; (3) the status of the immune system, with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and tiredness/loss of energy. Jaundice is not experienced in all cases; however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement can occur accompanied by right upper-quadrant pain. The acute phase of the disease can be accompanied by severe depression, meningitis, Guillain-Barre syndrome, myelitis, encephalitis, agranulocytosis, and/or thrombocytopenia.

Hepatitis B is generally self-limiting and will resolve in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life become carriers.

Hepatitis B virus (HBV) infects over 300 million people worldwide (Imperial, 1999, Gastroenterol. Hepatol., 14 (suppl), S1-5). In the United States, approximately 1.25 million individuals are chronic carriers of HBV as evidenced by the fact that they have measurable hepatitis B virus surface antigen HBsAg in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation is the only treatment option for patients with end-stage liver disease from HBV.

The natural progression of chronic HBV infection over a 10 to 20 year period leads to cirrhosis in 20-to-50% of patients and progression of HBV infection to hepatocellular carcinoma has been well documented. There have been no studies that have determined sub-populations that are most likely to progress to cirrhosis and/or hepatocellular carcinoma; thus all patients have equal risk of progression.

It is important to note that the survival for patients diagnosed with hepatocellular carcinoma is only 0.9 to 12.8 months from initial diagnosis (Takahashi et al., 1993, American Journal of Gastroenterology, 88, 240-243). Treatment of hepatocellular carcinoma with chemotherapeutic agents has not proven effective and only 10% of patients will benefit from surgery due to extensive tumor invasion of the liver (Trinchet et al., 1994, Presse Medicine, 23, 831-833). Given the aggressive nature of primary hepatocellular carcinoma, the only viable treatment alternative to surgery is liver transplantation (Pichlmayr et al., 1994, Hepatology., 20, 33S-40S).

Upon progression to cirrhosis, patients with chronic HBV infection present with clinical features that are common to clinical cirrhosis regardless of the initial cause (D'Amico et al., 1986, Digestive Diseases and Sciences, 31, 468-475). These clinical features can include: bleeding esophageal varices, ascites, jaundice, and encephalopathy (Zakim D, Boyer T D. Hepatology: A Textbook of Liver Disease, Second Edition, Volume 1, 1990, W. B. Saunders Company, Philadelphia). In the early stages of cirrhosis, patients are classified as compensated, meaning that although liver tissue damage has occurred, the patient's liver is still able to detoxify metabolites in the bloodstream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic- and viral-associated cirrhosis (D'Amico, supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% with bleeding, and 16% with encephalopathy. Hepatocellular carcinoma was observed in 6 (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and bleeding in 13% (D'Amico, supra).

Hepatitis B virus is a double-stranded circular DNA virus. It is a member of the Hepadnaviridae family. The virus is 42 nm in diameter, consisting of a central core that contains a core antigen (HBcAg) surrounded by an envelope containing a surface protein/surface antigen (HBsAg). It also contains an e antigen (HBeAg) that, along with HBcAg and HBsAg, is helpful in identifying this disease.

In HBV virions, the genome is found in an incomplete double-stranded form. HBV uses a reverse transcriptase to transcribe a positive-sense full-length RNA version of its genome back into DNA. This reverse transcriptase also contains DNA polymerase activity with which it begins replicating the newly-synthesized minus-sense DNA strand. However, it appears that the core protein encapsidates the reverse-transcriptase/polymerase before it completes replication.

From the free-floating form, the virus must first attach itself specifically to a host cell membrane. Viral attachment is one of the crucial steps that determine host and tissue specificity. Currently there are no in vitro cell lines that can be infected by HBV. There are, however, some cell lines, such as HepG2, which can support viral replication only upon transient or stable transfection using HBV DNA.

Cell Culture Models

As previously mentioned HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA could be co-transfected with ribozymes in cell culture. Such an approach has been used to report intracellular ribozyme activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV. In such cells, only the delivery of ribozymes would be required; however, a delivery screen would need to be performed.

Phenotypic Assays

Intracellular HBV gene expression can be assayed either by a Taqman® assay for HBV RNA or by ELISA for HBV protein. Extracellular virus can be assayed either by PCR for DNA or ELISA for protein. Antibodies are commercially available for HBV surface antigen and core protein. A secreted alkaline phosphatase expression plasmid can be used to normalize for differences in transfection efficiency and sample recovery.

Animal Models

There are several small animal models available to study HBV replication. One is the transplantation of HBV-infected liver tissue into irradiated mice. Viremia (as evidenced by measuring HBV DNA by PCR) is first detected 8 days after transplantation and peaks between 18- 25 days (Ilan et al., 1999, Hepatology, 29, 553-562).

Transgenic mice that express HBV have also been used as a model to evaluate potential anti-virals. HBV DNA is detectable in both liver and serum of the transgenic mice (Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen are also detected in the circulation of tumor-bearing mice (Yao et al., 1996, J. Viral Hepat., 3, 19-22).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose-dependent reduction in virus (50% reduction after two daily treatments at the highest dose) (Hurwitz et al., 1998. Antimicrob. Agents Chemother., 42, 2804-2809).

Therapeutic Approaches

Current therapeutic goals of treatment are three-fold: (1) to eliminate infectivity and transmission of HBV to others; (2) to arrest the progression of liver disease and improve the clinical prognosis; and (3) to prevent the development of hepatocellular carcinoma (HCC).

Interferon alpha (IFN-alpha) is the most common therapeutic for treating HBV infection; however, the FDA has recently approved Lamivudine (3TC®) as a therapeutic for treating chronic HBV infection. The standard duration of IFN-alpha therapy is 16 weeks; however, the optimal treatment length is still poorly defined. A complete response (where patients become both HBV DNA-negative and HbeAg-negative) occurs in approximately 25% of patients. Several factors have been identified that predict a favorable response to therapy, including: high ALT, low HBV DNA, being female, and heterosexual orientation.

There is also a risk of reactivation of the hepatitis B virus even after a successful response; this occurs in around 5% of responders and normally occurs within 1 year.

Side effects resulting from treatment with type 1 interferons can be divided into four general categories: (1) influenza-like symptoms, (2) neuropsychiatric side effects, (3) laboratory abnormalities, and (4) other miscellaneous side effects. Examples of influenza-like symptoms include, fatigue, fever, myalgia, malaise, appetite loss, tachycardia, rigors, headache and arthralgias. The influenza-like symptoms are usually short-lived and tend to abate after the first four weeks of dosing (Dusheiko et al., 1994, Journal of Viral Hepatitis, 1, 3-5). Neuropsychiatric side effects include irritability, apathy, mood changes, insomnia, cognitive changes, and depression. Laboratory abnormalities include the reduction of myeloid cells, including granulocytes, platelets, and, to a lesser extent, red blood cells. These changes in blood cell counts rarely lead to any significant clinical sequellae. In addition, increases in triglyceride concentrations and elevations in serum alaine and aspartate aminotransferase concentrations have been observed. Finally, thyroid abnormalities have been reported. These thyroid abnormalities are usually reversible after cessation of interferon therapy and can be controlled with appropriate medication during therapy. Miscellaneous side effects include nausea, diarrhea, abdominal and back pain, pruritus, alopecia, and rhinorrhea. In general, most side effects will abate after 4 to 8 weeks of therapy (Dushieko et al., supra).

Lamivudine (3TC®) is a nucleoside analogue, which is a very potent and specific inhibitor of HBV DNA synthesis, and has recently been approved for the treatment of chronic hepatitis B. Unlike treatment with interferon, treatment with 3TC® does not eliminate the HBV from the patient. Rather, viral replication is controlled and chronic administration results in improvements in liver histology in over 50% of patients. Phase III studies with 3TC®, showed that treatment for one year was associated with reduced liver inflammation and a delay in scarring of the liver. In addition, patients treated with 3TC® (100 mg per day) had a 98% reduction in hepatitis B DNA and a significantly higher rate of seroconversion, suggesting disease improvements after completion of therapy. However, cessation of therapy resulted in a reactivation of HBV replication in most patients. In addition, recent reports have documented 3TC® resistance in approximately 30% of patients.

Therefore, current therapies for treating HBV infection, including interferon and nucleoside analogues, such as IFN-alpha and 3TC®, are only partially effective. In addition, drug resistance to nucleoside analogues is now emerging, making treatment of chronic hepatitis B more difficult. Thus, a need exists for effective treatment of this disease that utilizes antiviral inhibitors that work by mechanisms other than those currently utilized in the treatment of both acute and chronic hepatitis B infections.

### SUMMARY OF THE INVENTION

This invention relates to compounds, compositions, and methods useful for modulating expression of genes, such as those genes associated with the development or maintenance of HBV infection, by RNA interference (RNAi) using short interfering nucleic acid (siNA). In particular, the instant invention features siNA molecules and methods to modulate the expression of HBV. A siNA of the invention can be unmodified or chemically modified. A siNA of the instant invention can be chemically synthesized, expressed from a vector, or enzymatically synthesized. The instant invention also features various chemically-modified synthetic short interfering nucleic acid (siNA) molecules capable of modulating HBV gene expression/activity in cells by RNA inference (RNAi). The use of chemically-modified siNA is expected to improve various properties of native siNA molecules through increased resistance to nuclease degradation in vivo and/or improved cellular uptake. The siNA molecules of the instant invention provide useful reagents and methods for a variety of therapeutic, diagnostic, target validation, genomic discovery, genetic engineering and pharmacogenomic applications.

In one embodiment, the invention features one or more siNA molecules and methods that independently or in combination modulate the expression of gene(s) encoding hepatitis B virus. Specifically, the present invention features siNA molecules that modulate the expression of HBV genes, for example genes encoding sequence referred to by Genbank Accession No. AB073834 or sequences referred to by Genbank Accession Nos. shown in Table I and/or homologous sequences thereof.

The description below of the various aspects and embodiments of the invention is provided with reference to the exemplary hepatitis B virus, including components or subunits thereof. However, the various aspects and embodiments are also directed to other genes that express other proteins associated with HBV infection, such as cellular proteins that are utilized in the HBV life-cycle. Those additional genes can be analyzed for target sites using the methods described for HBV herein. Thus, the inhibition and the effects of such inhibition of the other genes can be performed as described herein.

In one embodiment, the invention features a siNA molecule which down-regulates expression of a HBV gene, for example, wherein the HBV gene comprises HBV encoding sequence.

In one embodiment, the invention features a siNA molecule having RNAi activity against HBV RNA, wherein the siNA molecule comprises a sequence complementary to any RNA having HBV encoding sequence, for example sequence referred to by Genbank Accession No. AB073834 or sequences referred to by Genbank Accession Nos. in Table I and/or homologous sequences thereof.

In another embodiment, the invention features a siNA molecule comprising sequences selected from the group consisting of SEQ ID NOs.: 1-1524. In another embodiment, the invention features a siNA molecule having an antisense region complementary to any sequence having SEQ ID NOs.: 1-646. In another embodiment, the invention features a siNA molecule having an antisense region having any of SEQ ID NOs.: 647-1292, 1506, 1508, 1510, 1512, 1514, 1516, 1518, 1520, 1522, or 1524. In another embodiment, the invention features a siNA molecule having a sense region having any of SEQ ID NOs. 1-646, 1505, 1507, 1509, 1511, 1513, 1515, 1517, 1519, 1521, or 1523. The sequences shown in SEQ ID NOs.: 1-1524 are not limiting. A siNA molecule of the invention can comprise any contiguous HBV sequence (e.g., wherein the sense region of the siNA comprises about 19 contiguous HBV nucleotides and the antisense region comprises sequence complementary to about 19 contiguous HBV nucleotides). In yet another embodiment, the invention features a siNA molecule comprising a sequence, for example the antisense sequence of the siNA construct, complementary to a sequence or portion of sequence comprising Genbank Accession No. AB073834 or Genbank Accession Nos. in Table I and/or homologous sequences thereof.

Due to the high sequence variability of the HBV genome, selection of siNA molecules for broad therapeutic applications would likely involve the conserved regions of the HBV genome. Specifically, the present invention describes siNA molecules that target the conserved regions of the HBV genome.

In one embodiment, a siNA molecule of the invention has RNAi activity that modulates expression of RNA encoded by HBV genes, for example genes required for viral replication including genes required for HBV protein synthesis, such as the 5′-most 1500 nucleotides of the HBV pregenomic mRNA. This region controls the translational expression of the core protein (C), X protein (X), and DNA polymerase (P) genes, and plays a role in the replication of the viral DNA by serving as a template for reverse transcriptase. Disruption of this region in the RNA results in deficient protein synthesis as well as incomplete DNA synthesis (and inhibition of transcription from the defective genomes). Target sequences 5′ of the encapsidation site can result in the inclusion of the disrupted 3′ RNA within the core virion structure, and targeting sequences 3′ of the encapsidation site can result in the reduction in protein expression from both the 3′ and 5′ fragments. Alternative regions outside of the 5′-most 1500 nucleotides of the pregenomic mRNA also make suitable targets of siNA-mediated inhibition of HBV replication. Such targets include the mRNA regions that encode the viral S gene. Selection of particular target regions will depend upon the secondary structure of the pregenomic mRNA. Targets in the minor mRNAs can also be used, especially when folding or accessibility assays in these other RNAs reveal additional target sequences that are unavailable in the pregenomic mRNA species. A desirable target in the pregenomic RNA is a proposed bipartite stem-loop structure in the 3′-end of the pregenomic RNA that is believed to be critical for viral replication (Kidd and Kidd-Ljunggren, 1996. Nuc. Acid Res. 24:3295-3302). The 5′-end of the HBV pregenomic RNA carries a cis-acting encapsidation signal, which has inverted repeat sequences that are thought to form a bipartite stem-loop structure. Due to a terminal redundancy in the pregenomic RNA, the putative stem-loop also occurs at the 3′-end. While it is the 5′ copy that functions in polymerase binding and encapsidation, reverse transcription actually begins from the 3′ stem-loop. To start reverse transcription, a 4 nucleotide primer that is covalently attached to the polymerase is made, using a bulge in the 5′ encapsidation signal as template. This primer is then shifted, by an unknown mechanism, to the DRI primer binding site in the 3′ stem-loop structure, and reverse transcription proceeds from that point. The 3′ stem-loop, and especially the DRI primer binding site, appear to be highly effective targets for siNA mediated intervention. Sequences of the pregenomic RNA are shared by the mRNAs for surface, core, polymerase, and X proteins. Due to the overlapping nature of the HBV transcripts, all share a common 3′-end. Therefore, siNA targeting of this common 3′-end will thus disrupt the pregenomic RNA as well as all of the mRNAs for surface, core, polymerase and X proteins.

In one embodiment of the invention a siNA molecule is adapted for use to treat human hepatitis B virus infections, which include productive virus infection, latent or persistent virus infection, and HBV-induced hepatocyte transformation. The utility can be extended to other species of HBV that infect non-human animals where such infections are of veterinary importance. A siNA molecule can comprise a sense region and an antisense region, wherein said antisense region can comprise sequence complementary to an RNA sequence encoding HBV and the sense region can comprise sequence complementary to the antisense region. A siNA molecule can be assembled from two nucleic acid fragments wherein one fragment can comprise the sense region and the second fragment can comprise the antisense region of said siNA molecule. The sense region and antisense region can be covalently connected via a linker molecule. The linker molecule can be a polynucleotide or non-nucleotide linker. The sense region of a siNA molecule of the invention can comprise a 3′-terminal overhang and the antisense region can comprise a 3′-terminal overhang. The 3′-terminal overhangs each can comprise about 2 nucleotides. The antisense region 3′-terminal nucleotide overhang can be complementary to RNA encoding HBV. The sense region can comprise a terminal cap moiety at the 3′-end, 5′-end, and/or both the 3′ and the 5′-ends of the sense region. The antisense region can also comprise a terminal cap moiety at the 3′-end, 5′-end, and/or both the 3′ and the 5′-ends of the antisense region.

In one embodiment, the invention features one or more siNA molecules and methods that independently or in combination modulate the expression of genes representing cellular targets for HBV infection, such as cellular receptors, cell surface molecules, cellular enzymes, cellular transcription factors, and/or cytokines, second messengers, and cellular accessory molecules including but not limited to interferon regulatory factors (IRFs such as Genbank Accession No. AF082503.1), cellular PKR protein kinase (such as Genbank Accession No. XM_{—}002661.7), human eukaryotic initiation factors 2B (elF2Bgamma, such as Genbank Accession No. AF256223 and/or elF2gamma, such as Genbank Accession No. NM_{—}006874.1), human DEAD Box protein DDX3 (such as Genbank Accession No. XM_{—}018021.2), and cellular proteins that are essential for the maintenance of persistent infection of hepatocytes, such as proteins that interact with the HBV-encoded HBx regulatory protein.

In one embodiment, nucleic acid molecules of the invention that act as mediators of the RNA interference gene silencing response are double-stranded nucleic acid molecules. In another embodiment, the siNA molecules of the invention consist of duplexes containing about 19 base pairs between oligonucleotides comprising about 19 to about 25 nucleotides. In yet another embodiment, siNA molecules of the invention comprise duplexes with overhanging ends of about 1-3 (e.g., about 1, 2, or 3) nucleotides, for example about 21-nucleotide duplexes with about 19 base pairs and a 3′-terminal mononucleotide, dinucleotide, or trinucleotide overhang.

In one embodiment, the invention features one or more chemically-modified siNA constructs having specificity for HBV expressing nucleic acid molecules. Non-limiting examples of such chemical modifications include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds. Furthermore, contrary to the data published by Parrish et al., supra, applicant demonstrates that multiple (greater than one) phosphorothioate substitutions are well-tolerated and confer substantial increases in serum stability for modified siNA constructs.

The antisense region of a siNA molecule of the invention can comprise a phosphorothioate internucleotide linkage at the 3′-end of said antisense region. The antisense region can comprise between about one and about five phosphorothioate internucleotide linkages at the 5′-end of said antisense region. The 3′-terminal nucleotide overhangs of a siNA molecule of the invention can comprise ribonucleotides or deoxyribonucleotides that are chemically modified at a nucleic acid sugar, base, or backbone. The 3′-terminal nucleotide overhangs can comprise one or more universal base ribonucleotides. The 3′-terminal nucleotide overhangs can comprise one or more acyclic nucleotides.

In a non-limiting example, the introduction of chemically-modified nucleotides into nucleic acid molecules will provide a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example when compared to an all RNA nucleic acid molecule, the overall activity of the modified nucleic acid molecule can be greater than the native molecule due to improved stability and/or delivery of the molecule. Unlike native unmodified siNA, chemically-modified siNA can also minimize the possibility of activating interferon activity in humans.

One embodiment of the invention provides an expression vector comprising a nucleic acid sequence encoding at least one siNA molecule of the invention in a manner that allows expression of the nucleic acid molecule. Another embodiment of the invention provides a mammalian cell comprising such an expression vector. The mammalian cell can be a human cell. The siNA molecule of the expression vector can comprise a sense region and an antisense region and the antisense region can comprise sequence complementary to a RNA sequence encoding HBV and the sense region can comprise sequence complementary to the antisense region. The siNA molecule can comprise two distinct strands having complementary sense and antisense regions. The siNA molecule can comprise a single strand having complementary sense and antisense regions.

In one embodiment, the invention features a chemically-modified short interfering nucleic acid (siNA) molecule capable of mediating RNA interference (RNAi) against HBV inside a cell or reconstituted in vitro system, wherein the chemical modification comprises one or more (e.g., about 1, 2, 3, 4, 5, 6, 7, 8, 9, 10, or more) nucleotides comprising a backbone modified internucleotide linkage having Formula I:

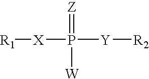

wherein each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y, and Z are optionally not all O.

The chemically-modified internucleotide linkages having Formula I, for example wherein any Z, W, X, and/or Y independently comprises a sulphur atom, can be present in one or both oligonucleotide strands of the siNA duplex, for example in the sense strand, the antisense strand, or both strands. The siNA molecules of the invention can comprise one or more (e.g., about 1, 2, 3, 4, 5, 6, 7, 8, 9, 10, or more) chemically-modified internucleotide linkages having Formula I at the 3′-end, the 5′-end, or both of the 3′- and 5′-ends of the sense strand, the antisense strand, or both strands. For example, an exemplary siNA molecule of the invention can comprise between about 1 and about 5 or more (e.g., about 1, 2, 3, 4, 5, or more) chemically-modified internucleotide linkages having Formula I at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine nucleotides with chemically-modified internucleotide linkages having Formula I in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine nucleotides with chemically-modified internucleotide linkages having Formula I in the sense strand, the antisense strand, or both strands. In another embodiment, a siNA molecule of the invention having internucleotide linkage(s) of Formula I also comprises a chemically-modified nucleotide or non-nucleotide having any of Formulae I-VII.

In one embodiment, the invention features a chemically-modified short interfering nucleic acid (siNA) molecule capable of mediating RNA interference (RNAi) against HBV inside a cell or reconstituted in vitro system, wherein the chemical modification comprises one or more (e.g., about 1, 2, 3, 4, 5, 6, 7, 8, 9, 10, or more) nucleotides or non-nucleotides having Formula II:

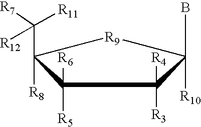

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula II can be present in one or both oligonucleotide strands of the siNA duplex, for example in the sense strand, the antisense strand, or both strands. The siNA molecules of the invention can comprise one or more chemically-modified nucleotide or non-nucleotide of Formula II at the 3′-end, the 5′-end, or both of the 3′- and 5′-ends of the sense strand, the antisense strand, or both strands. For example, an exemplary siNA molecule of the invention can comprise between about 1 and about 5 or more (e.g., about 1, 2, 3, 4, 5, or more) chemically-modified nucleotide or non-nucleotide of Formula II at the 5′-end of the sense strand, the antisense strand, or both strands. In anther non-limiting example, an exemplary siNA molecule of the invention can comprise between about 1 and about 5 or more (e.g., about 1, 2, 3, 4, 5, or more) chemically-modified nucleotide or non-nucleotide of Formula II at the 3′-end of the sense strand, the antisense strand, or both strands.

In one embodiment, the invention features a chemically-modified short interfering nucleic acid (siNA) molecule capable of mediating RNA interference (RNAi) against HBV inside a cell or reconstituted in vitro system, wherein the chemical modification comprises one or more (e.g., about 1, 2, 3, 4, 5, 6, 7, 8, 9, 10, or more) nucleotides or non-nucleotides having Formula III:

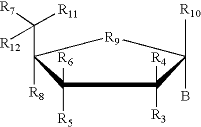

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula III can be present in one or both oligonucleotide strands of the siNA duplex, for example in the sense strand, the antisense strand, or both strands. The siNA molecules of the invention can comprise one or more chemically-modified nucleotide or non-nucleotide of Formula III at the 3′-end, the 5′-end, or both of the 3′- and 5′-ends of the sense strand, the antisense strand, or both strands. For example, an exemplary siNA molecule of the invention can comprise between about 1 and about 5 or more (e.g., about 1, 2, 3, 4, 5, or more) chemically-modified nucleotide or non-nucleotide of Formula III at the 5′-end of the sense strand, the antisense strand, or both strands. In anther non-limiting example, an exemplary siNA molecule of the invention can comprise between about 1 and about 5 or more (e.g., about 1, 2, 3, 4, 5, or more) chemically-modified nucleotide or non-nucleotide of Formula III at the 3′-end of the sense strand, the antisense strand, or both strands.

In another embodiment, a siNA molecule of the invention comprises a nucleotide having Formula II or III, wherein the nucleotide having Formula II or III is in an inverted configuration. For example, the nucleotide having Formula II or III is connected to the siNA construct in a 3′,3′; 3′-2′, 2′-3′; or 5′,5′ configuration, such as at the 3′-end, 5′-end, or both 3′ and 5′-ends of one or both siNA strands.

In one embodiment, the invention features a chemically-modified short interfering nucleic acid (siNA) molecule capable of mediating RNA interference (RNAi) against HBV inside a cell or reconstituted in vitro system, wherein the chemical modification comprises a 5′-terminal phosphate group having Formula IV:

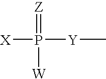

wherein each X and Y is independently O, S, N, alkyl, substituted alkyl, or alkylhalo; each Z and W is independently O, S, N, alkyl, substituted alkyl, O-alkyl, S-alkyl, alkaryl, aralkyl, or alkylhalo; and wherein W, X, Y and Z are not all O.

In one embodiment, the invention features a siNA molecule having a 5′-terminal phosphate group having Formula IV on the target-complementary strand, for example a strand complementary to a target RNA, wherein the siNA molecule comprises an all RNA siNA molecule. In another embodiment, the invention features a siNA molecule having a 5′-terminal phosphate group having Formula IV on the target-complementary strand wherein the siNA molecule also comprises 1-3 (e.g., 1, 2, or 3) nucleotide 3′-terminal nucleotide overhangs having between about 1 and about 4 (e.g., about 1, 2, 3, or 4) deoxyribonucleotides on the 3′-end of one or both strands. In another embodiment, a 5′-terminal phosphate group having Formula IV is present on the target-complementary strand of a siNA molecule of the invention, for example a siNA molecule having chemical modifications having any of Formulae I-VII.

In one embodiment, the invention features a chemically-modified short interfering nucleic acid (siNA) molecule capable of mediating RNA interference (RNAi) against HBV inside a cell or reconstituted in vitro system, wherein the chemical modification comprises one or more phosphorothioate internucleotide linkages. For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′- and 5′-ends of the sense strand, the antisense strand, or both strands. For example, an exemplary siNA molecule of the invention can comprise between about 1 and about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the antisense strand, or both strands.

In one embodiment, the invention features a siNA molecule, wherein the sense strand comprises one or more, for example about 1, 2, 3, 4, 5, 6, 7, 8, 9, 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base-modified nucleotides, and optionally a terminal cap molecule at the 3′-end, the 5′-end, or both of the 3′- and 5′-ends of the sense strand; and wherein the antisense strand comprises any of between 1 and 10 or more, specifically about 1, 2, 3, 4, 5, 6, 7, 8, 9, 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base-modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more, pyrimidine nucleotides of the sense and/or antisense siNA stand are chemically modified with 2′-deoxy, 2′-O-methyl and/or 2′-deoxy-2′-fluoro nucleotides, with or without one or more, for example about 1, 2, 3, 4, 5, 6, 7, 8, 9, 10 or more phosphorothioate internucleotide linkages and/or a terminal cap molecule at the 3′-end, the 5′-end, or both of the 3′- and 5′-ends, being present in the same or different strand.

In another embodiment, the invention features a siNA molecule, wherein the sense strand comprises between about 1 and about 5, specifically about 1, 2, 3, 4, or 5 phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base-modified nucleotides, and optionally a terminal cap molecule at the 3′-end, the 5′-end, or both of the 3′- and 5′-ends of the sense strand; and wherein the antisense strand comprises any of between about 1 and about 5 or more, specifically about 1, 2, 3, 4, 5, or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base-modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense siNA stand are chemically modified with 2′-deoxy, 2′-O-methyl and/or 2′-deoxy-2′-fluoro nucleotides, with or without between about 1 and about 5 or more, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In one embodiment, the invention features a siNA molecule, wherein the antisense strand comprises one or more, for example about 1, 2, 3, 4, 5, 6, 7, 8, 9, 10 or more phosphorothioate internucleotide linkages, and/or between one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base-modified nucleotides, and optionally a terminal cap molecule at the 3′-end, the 5′-end, or both of the 3′- and 5′-ends of the sense strand; and wherein the antisense strand comprises any of between about 1 and about 10, specifically about 1, 2, 3, 4, 5, 6, 7, 8, 9, 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base-modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense siNA stand are chemically modified with 2′-deoxy, 2′-O-methyl and/or 2′-deoxy-2′-fluoro nucleotides, with or without one or more, for example about 1, 2, 3, 4, 5, 6, 7, 8, 9, 10 or more phosphorothioate internucleotide linkages and/or a terminal cap molecule at the 3′-end, the 5′-end, or both of the 3′- and 5′-ends, being present in the same or different strand.

In another embodiment, the invention features a siNA molecule, wherein the antisense strand comprises between about 1 and about 5 or more, specifically about 1, 2, 3, 4, 5 or more phosphorothioate internucleotide linkages, and/or one or more (e.g., about 1, 2, 3, 4, 5 or more) 2′-deoxy, 2′-O-methyl, 2′-deoxy-2′-fluoro, and/or one or more (e.g., about 1, 2, 3, 4, 5 or more) universal base-modified nucleotides, and optionally a terminal cap molecule at the 3′-end, the 5′-end, or both of the 3′- and 5′-ends of the sense strand; and wherein the antisense strand comprises any of between about 1 and about 5 or more, specifically about 1, 2, 3, 4, 5 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base-modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense siNA stand are chemically modified with 2′-deoxy, 2′-O-methyl and/or 2′-deoxy-2′-fluoro nucleotides, with or without between about 1 and about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In one embodiment, the invention features a chemically-modified short interfering nucleic acid (siNA) molecule having between about 1 and about 5, specifically about 1, 2, 3, 4, 5 or more phosphorothioate internucleotide linkages in each strand of the siNA molecule.

In another embodiment, the invention features a siNA molecule comprising 2′-5′ internucleotide linkages. The 2′-5′ internucleotide linkage(s) can be at 3′-end the 5′-end, the 3′-end, or both of the 5′- and 3′-ends of one or both siNA sequence strands. In addition, the 2′-5′ internucleotide linkage(s) can be present at various other positions within one or both siNA sequence strands, for example, about 1, 2, 3, 4, 5, 6, 7, 8, 9, 10, or more including every internucleotide linkage of a pyrimidine nucleotide in one or both strands of the siNA molecule can comprise a 2′-5′ internucleotide linkage, or about 1, 2, 3, 4, 5, 6, 7, 8, 9, 10, or more including every internucleotide linkage of a purine nucleotide in one or both strands of the siNA molecule can comprise a 2′-5′ internucleotide linkage.

In another embodiment, a chemically-modified siNA molecule of the invention comprises a duplex having two strands, one or both of which can be chemically modified, wherein each strand is between about 18 and about 27 (e.g., about 18, 19, 20, 21, 22, 23, 24, 25, 26, or 27) nucleotides in length, wherein the duplex has between about 18 and about 23 (e.g., about 18, 19, 20, 21, 22, or 23) base pairs, and wherein the chemical modification comprises a structure having any of Formulae I-VII. For example, an exemplary chemically-modified siNA molecule of the invention comprises a duplex having two strands, one or both of which can be chemically modified with a chemical modification having any of Formulae I-VII, wherein each strand consists of about 21 nucleotides, each having two 2-nucleotide 3′-terminal nucleotide overhangs, and wherein the duplex has 19 base pairs.

In another embodiment, a siNA molecule of the invention comprises a single-stranded hairpin structure, wherein the siNA is between about 36 and about 70 (e.g., about 36, 40, 45, 50, 55, 60, 65, or 70) nucleotides in length having between about 18 and about 23 (e.g., about 18, 19, 20, 21, 22, or 23) base pairs, and wherein the siNA can include a chemical modification comprising a structure having any of Formulae I-VII. For example, an exemplary chemically-modified siNA molecule of the invention comprises a linear oligonucleotide having between about 42 and about 50 (e.g., about 42, 43, 44, 45, 46, 47, 48, 49, or 50) nucleotides that is chemically modified with a chemical modification having any of Formulae I-VII, wherein the linear oligonucleotide forms a hairpin structure having 19 base pairs and a 2 nucleotide 3′-terminal nucleotide overhang.

In another embodiment, a linear hairpin siNA molecule of the invention contains a stem loop motif, wherein the loop portion of the siNA molecule is biodegradable. For example, a linear hairpin siNA molecule of the invention is designed such that degradation of the loop portion of the siNA molecule in vivo can generate a double-stranded siNA molecule with 3′-terminal overhangs, such as 3′-terminal nucleotide overhangs comprising about 2 nucleotides.

In another embodiment, a siNA molecule of the invention comprises a circular nucleic acid molecule, wherein the siNA is between about 38 and about 70 (e.g., about 38, 40, 45, 50, 55, 60, 65, or 70) nucleotides in length having between about 18 and about 23 (e.g., about 18, 19, 20, 21, 22, or 23) base pairs, and wherein the siNA can include a chemical modification, which comprises a structure having any of Formulae I-VII. For example, an exemplary chemically-modified siNA molecule of the invention comprises a circular oligonucleotide having between about 42 and about 50 (e.g., about 42, 43, 44, 45, 46, 47, 48, 49, or 50) nucleotides that is chemically modified with a chemical modification having any of Formulae I-VII, wherein the circular oligonucleotide forms a dumbbell-shaped structure having 19 base pairs and 2 loops.

In another embodiment, a circular siNA molecule of the invention contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

In one embodiment, a siNA molecule of the invention comprises at least one (e.g., about 1, 2, 3, 4, 5, 6, 7, 8, 9, 10, or more) abasic moiety, for example a compound having Formula V:

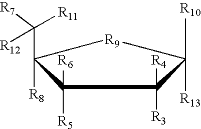

wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2.

In one embodiment, a siNA molecule of the invention comprises at least one (e.g., about 1, 2, 3, 4, 5, 6, 7, 8, 9, 10, or more) inverted abasic moiety, for example a compound having Formula VI:

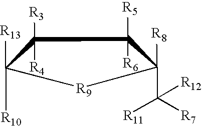

wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 serve as points of attachment to the siNA molecule of the invention.

In another embodiment, a siNA molecule of the invention comprises at least one (e.g., about 1, 2, 3, 4, 5, 6, 7, 8, 9, 10, or more) substituted polyalkyl moieties, for example a compound having Formula VII:

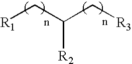

wherein each n is independently an integer from 1 to 12, R1, R2 and R3 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I, and either R1, R2 or R3 serve as points of attachment to the siNA molecule of the invention.

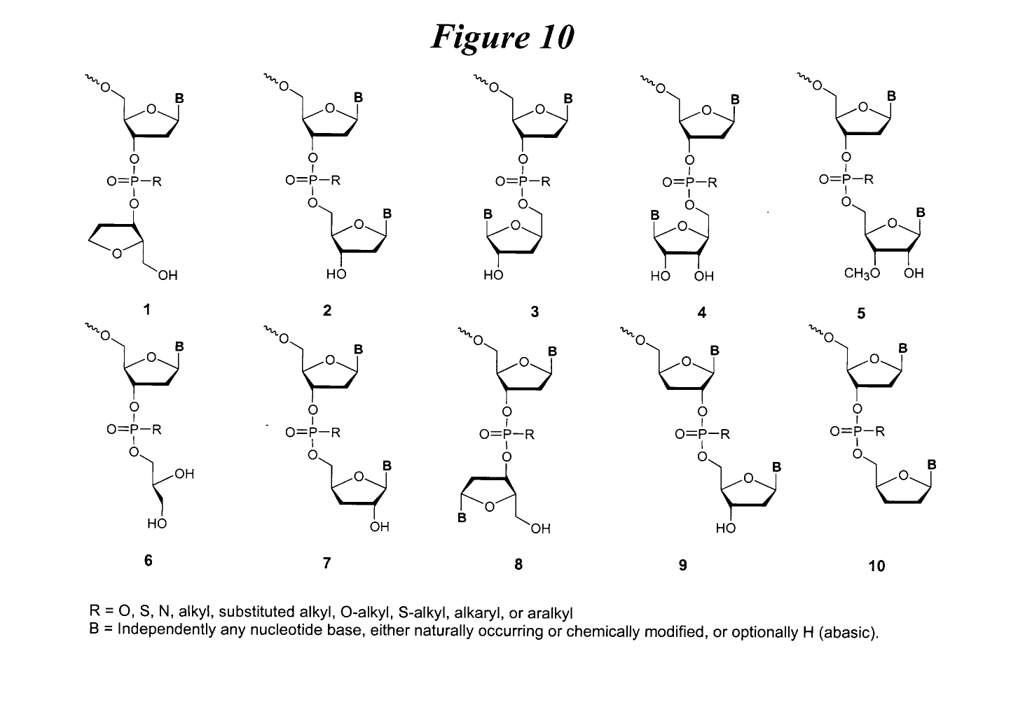

In another embodiment, the invention features a compound having Formula VII, wherein R1 and R2 are hydroxyl (OH) groups, n=1, and R3 comprises O and is the point of attachment to the 3′-end, 5-end, or both 3′ and 5′-ends of one or both strands of a double-stranded siNA molecule of the invention or to a single-stranded siNA molecule of the invention. This modification is referred to herein as “glyceryl” (for example modification 6 in FIG. 10).

In another embodiment, a moiety having any of Formula V, VI or VII of the invention is at the 3′-end, the 5′-end, or both of the 3′- and 5′-ends of a siNA molecule of the invention. For example, a moiety having Formula V, VI or VII can be present at the 3′-end, the 5′-end, or both of the 3′- and 5′-ends of the antisense strand, the sense strand, or both the antisense and sense strands of the siNA molecule. In addition, a moiety having Formula VII can be present at the 3′-end or the 5′-end of a hairpin siNA molecule as described herein.

In another embodiment, a siNA molecule of the invention comprises an abasic residue having Formula V or VI, wherein the abasic residue having Formula V or VI is connected to the siNA construct in a 3′-3′, 3′-2′, 2′-3′, or 5′-5′ configuration, such as at the 3′-end, 5′-end, or both 3′ and ′5′-ends of one or both siNA strands.

In one embodiment, a siNA molecule of the invention comprises one or more (e.g., about 1, 2, 3, 4, 5, 6, 7, 8, 9, 10, or more) locked nucleic acid (LNA) nucleotides, for example at the 5′-end, 3′-end, 5′ and 3′-end, or any combination thereof, of the siNA molecule.

In another embodiment, a siNA molecule of the invention comprises one or more (e.g., about 1, 2, 3, 4, 5, 6, 7, 8, 9, 10, or more) acyclic nucleotides, for example at the 5′-end, 3′-end, 5′ and 3′-end, or any combination thereof, of the siNA molecule.

In one embodiment, the invention features a chemically-modified short interfering nucleic acid (siNA) molecule of the invention capable of mediating RNA interference (RNAi) against HBV inside a cell or reconstituted in vitro system, wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides).

In one embodiment, the invention features a chemically-modified short interfering nucleic acid (siNA) molecule of the invention capable of mediating RNA interference (RNAi) against HBV inside a cell or reconstituted in vitro system, wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), wherein any nucleotides comprising a 3′-terminal nucleotide overhang that are present in said sense region are 2′-deoxy nucleotides.

In one embodiment, the invention features a chemically-modified short interfering nucleic acid (siNA) molecule of the invention capable of mediating RNA interference (RNAi) against HBV inside a cell or reconstituted in vitro system, wherein the chemically-modified siNA comprises an antisense region, where any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides).

In one embodiment, the invention features a chemically-modified short interfering nucleic acid (siNA) molecule of the invention capable of mediating RNA interference (RNAi) against HBV inside a cell or reconstituted in vitro system, wherein the chemically-modified siNA comprises an antisense region, where any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), wherein any nucleotides comprising a 3′-terminal nucleotide overhang that are present in said antisense region are 2′-deoxy nucleotides.

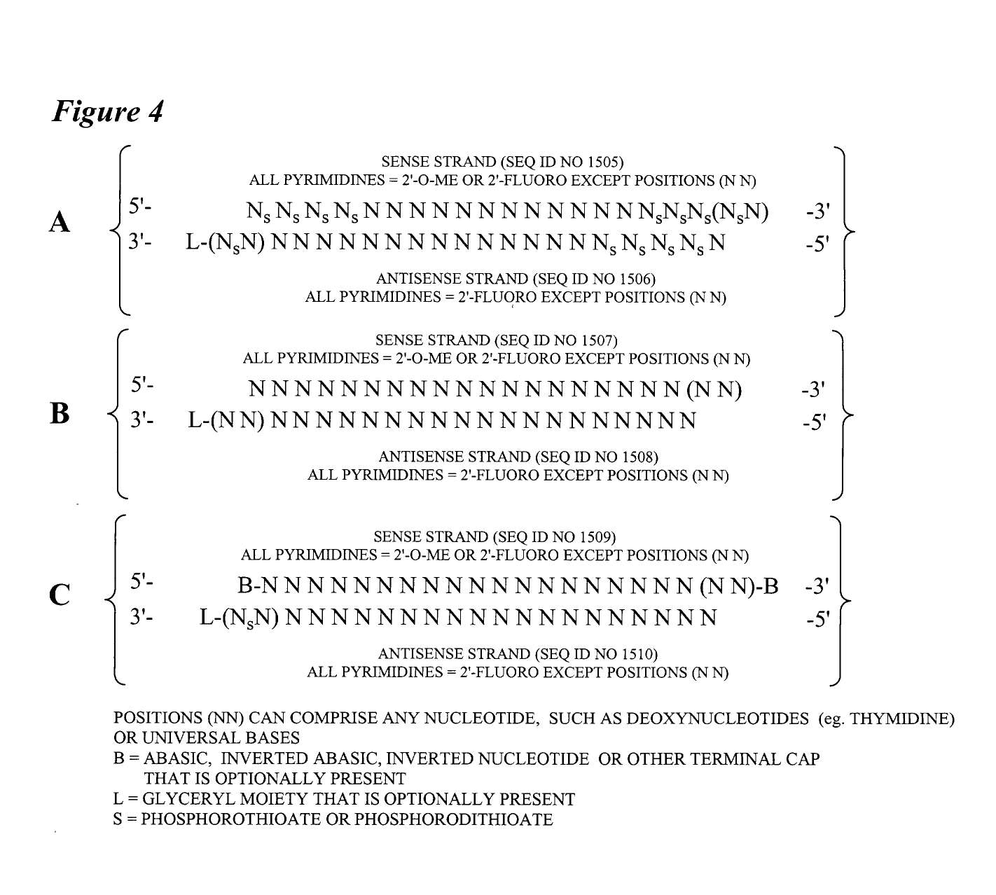

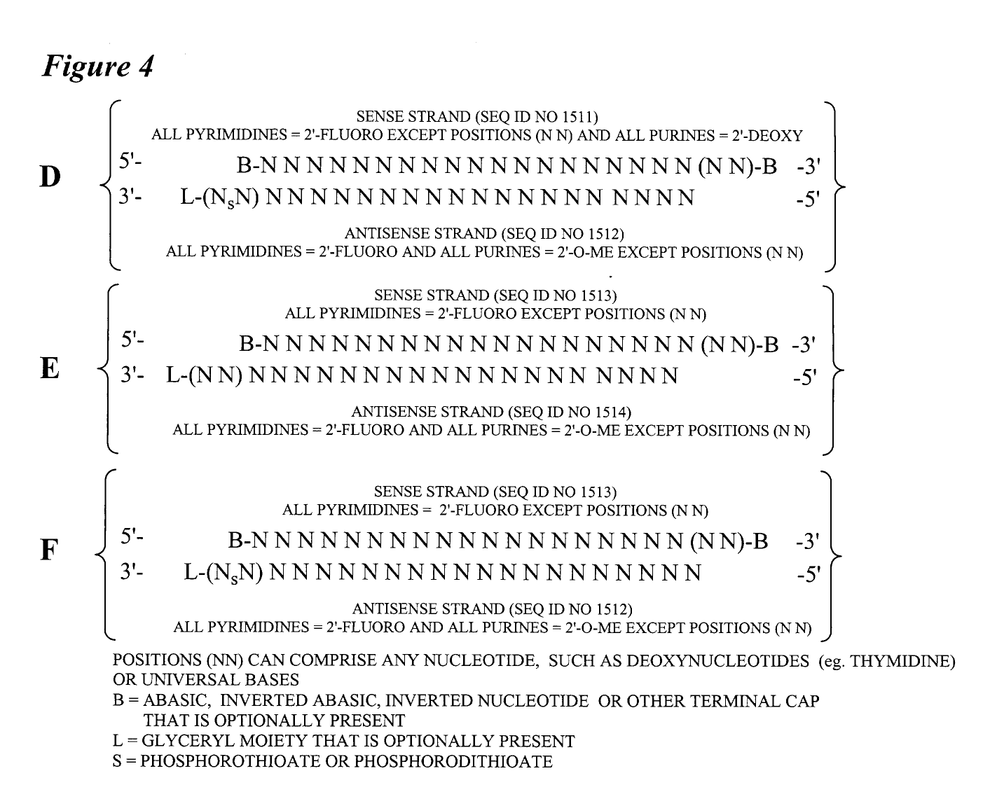

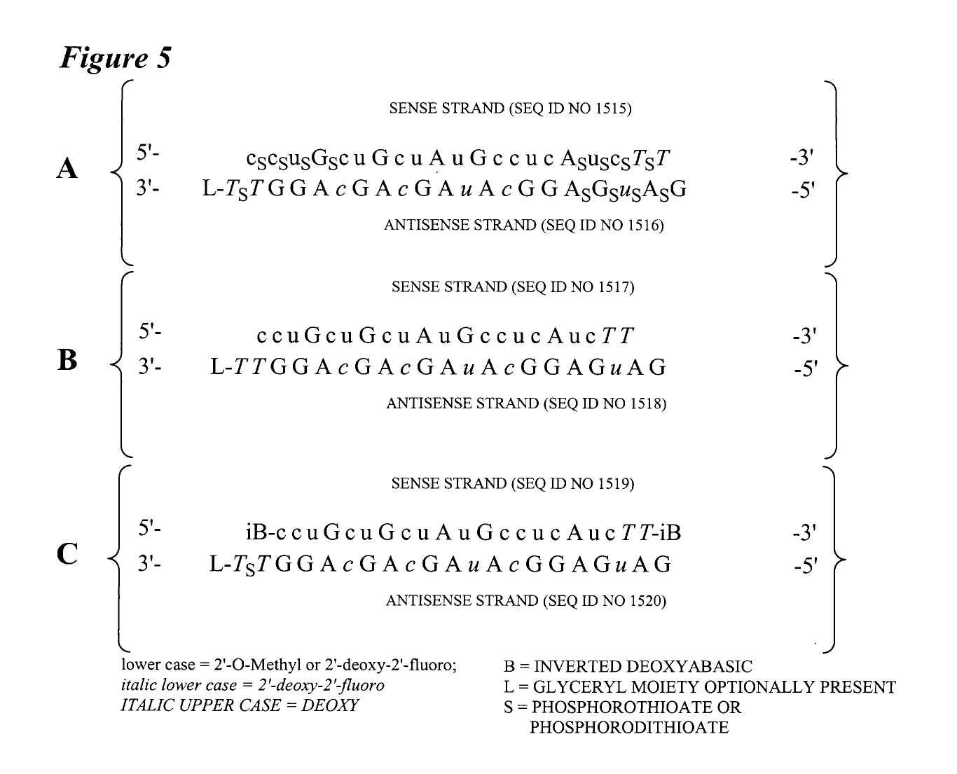

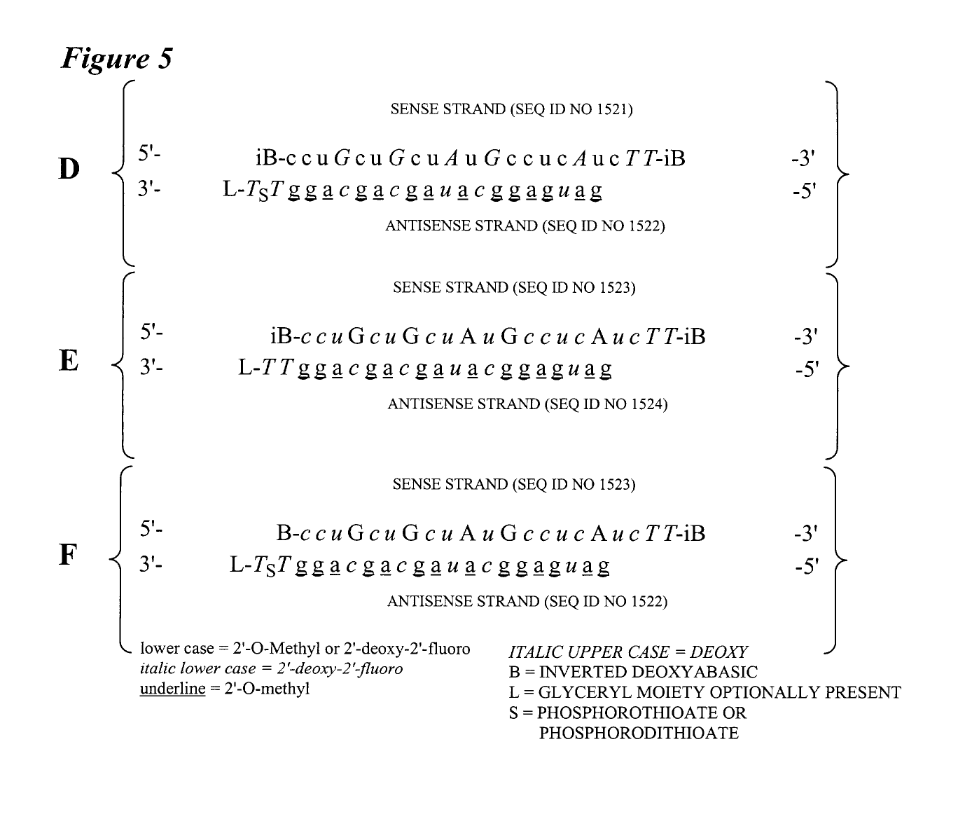

In one embodiment, the invention features a chemically-modified short interfering nucleic acid (siNA) molecule of the invention capable of mediating RNA interference (RNAi) against HBV inside a cell or reconstituted in vitro system, wherein the chemically-modified siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), and inverted deoxy abasic modifications that are optionally present at the 3′-end, the 5′-end, or both of the 3′- and 5′-ends of the sense region, the sense region optionally further comprising a 3′-terminal nucleotide overhang having between about 1 and about 4 (e.g, about 1, 2, 3, or 4) 2′-deoxyribonucleotides; and wherein the chemically-modified short interfering nucleic acid molecule comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein or shown in FIG. 10, that is optionally present at the 3′-end, the 5′-end, or both of the 3′- and 5′-ends of the antisense sequence, the antisense region optionally further comprising a 3′-terminal overhang having between about 1 and about 4 (e.g, about 1, 2, 3, or 4) 2′-deoxynucleotides, wherein the overhang nucleotides can further comprise one or more (e.g., 1, 2, 3, or 4 ) phosphorothioate internucleotide linkages. Non-limiting examples of these chemically-modified siNAs are shown in FIGS. 4 and 5 (SEQ ID NOs.: 396/397 and 406/407) herein.

In one embodiment, the invention features a chemically-modified short interfering nucleic acid (siNA) molecule of the invention capable of mediating RNA interference (RNAi) against HBV inside a cell or reconstituted in vitro system, wherein the siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are purine ribonucleotides (e.g., wherein all purine nucleotides are purine ribonucleotides or alternately a plurality of purine nucleotides are purine ribonucleotides), and inverted deoxy abasic modifications that are optionally present at the 3′-end, the 5′-end, or both of the 3′- and 5′-ends of the sense region, the sense region optionally further comprising a 3′-terminal nucleotide overhang having between about 1 and about 4 (e.g, about 1, 2, 3, or 4) 2′-deoxyribonucleotides; and wherein the siNA comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein or shown in FIG. 10, that is optionally present at the 3′-end, the 5′-end, or both of the 3′- and 5′-ends of the antisense sequence, the antisense region optionally further comprising a 3′-terminal nucleotide overhang having between about 1 and about 4 (e.g, about 1, 2, 3, or 4) 2′-deoxynucleotides, wherein the overhang nucleotides can further comprise one or more (e.g., 1, 2, 3, or 4) phosphorothioate internucleotide linkages. Non-limiting examples of these chemically-modified siNAs are shown in FIGS. 4 and 5 (SEQ ID NOs.: 398/397 and 408/407) herein.

In one embodiment, the invention features a chemically-modified short interfering nucleic acid molecule (siNA) capable of mediating RNA interference (RNAi) against HBV inside a cell or reconstituted in vitro system, wherein the chemical modification comprises a conjugate covalently attached to the chemically-modified siNA molecule. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached to both the 3′-end and the 5′-end of the sense strand, the antisense strand, or both strands of the chemically-modified siNA molecule, or any combination thereof. In one embodiment, a conjugate molecule of the invention comprises a molecule that facilitates delivery of a chemically-modified siNA molecule molecule into a biological system such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese et al., U.S. Ser. No. 60/311,865, incorporated by reference herein.

In one embodiment, the invention features a short interfering nucleic acid (siNA) molecule capable of mediating RNA interference (RNAi) against HBV inside a cell or reconstituted in vitro system, wherein one or both strands of the siNA molecule that are assembled from two separate oligonucleotides comprise ribonucleotides at positions within the siNA that are critical for siNA mediated RNAi in a cell. All other positions within the siNA can include chemically-modified nucleotides and/or non-nucleotides such as nucleotides and or non-nucleotides having any of Formulae I-VII or any combination thereof to the extent that the ability of the siNA molecule to support RNAi activity in a cell is maintained.

In one embodiment, the invention features a short interfering nucleic acid (siNA) molecule capable of mediating RNA interference (RNAi) against HBV inside a cell or reconstituted in vitro system, wherein neither of the strands of the siNA molecule that are assembled from two separate oligonucleotides comprise ribonucleotides that are critical for siNA mediated RNAi in a cell. For example, all the positions within the siNA molecule can include chemically-modified nucleotides and/or non-nucleotides such as nucleotides and or non-nucleotides having any of Formulae I-VII or any combination thereof to the extent that the ability of the siNA molecule to support RNAi activity in a cell is maintained.

In one embodiment, the invention features a short interfering nucleic acid (siNA) molecule capable of mediating RNA interference (RNAi) against HBV inside a cell or reconstituted in vitro system, wherein the antisense region and/or the sense region of the siNA molecule comprise ribonucleotides at positions within the siNA that are critical for siNA mediated RNAi in a cell. All other positions within the siNA can include chemically-modified nucleotides and/or non-nucleotides such as nucleotides and or non-nucleotides having any of Formulae I-VII or any combination thereof to the extent that the ability of the siNA molecule to support RNAi activity in a cell is maintained.

In one embodiment, the invention features a short interfering nucleic acid (siNA) molecule capable of mediating RNA interference (RNAi) against HBV inside a cell or reconstituted in vitro system, wherein wherein the antisense region and/or the sense region of the siNA molecule that are assembled from two separate oligonucleotides comprise ribonucleotides that are critical for siNA mediated RNAi in a cell. For example, all the positions within the siNA molecule can include chemically-modified nucleotides and/or non-nucleotides such as nucleotides and or non-nucleotides having any of Formulae I-VII or any combination thereof to the extent that the ability of the siNA molecule molecule to support RNAi activity in a cell is maintained.

In one embodiment, the invention features a method for modulating the expression of a HBV gene within a cell, comprising: (a) synthesizing a siNA molecule of the invention, which can be chemically modified, wherein one of the siNA strands includes a sequence complementary to RNA of the HBV gene; and (b) introducing the siNA molecule into a cell under conditions suitable to modulate the expression of the HBV gene in the cell.

In one embodiment, the invention features a method for modulating the expression of a HBV gene within a cell, comprising: (a) synthesizing a siNA molecule of the invention, which can be chemically modified, wherein one of the siNA strands includes a sequence complementary to RNA of the HBV gene and wherein the sense strand sequence of the siNA is identical to the complementary sequence of the target RNA; and (b) introducing the siNA molecule into a cell under conditions suitable to modulate the expression of the HBV gene in the cell.

In another embodiment, the invention features a method for modulating the expression of more than one HBV gene within a cell, comprising: (a) synthesizing siNA molecules of the invention, which can be chemically modified, wherein one of the siNA strands includes a sequence complementary to RNA of the HBV genes; and (b) introducing the siNA molecules into a cell under conditions suitable to modulate the expression of the HBV genes in the cell.

In another embodiment, the invention features a method for modulating the expression of more than one HBV gene within a cell, comprising: (a) synthesizing a siNA molecule of the invention, which can be chemically modified, wherein one of the siNA strands includes a sequence complementary to RNA of the HBV gene and wherein the sense strand sequence of the siNA is identical to the complementary sequence of the target RNA; and (b) introducing the siNA molecules into a cell under conditions suitable to modulate the expression of the HBV genes in the cell.

In one embodiment, the invention features a method of modulating the expression of a HBV gene in a tissue explant, comprising: (a) synthesizing a siNA molecule of the invention, which can be chemically modified, wherein one of the siNA strands includes a sequence complementary to RNA of the HBV gene; (b) introducing the siNA molecule into a cell of the tissue explant derived from a particular organism under conditions suitable to modulate the expression of the HBV gene in the tissue explant, and (c) optionally introducing the tissue explant back into the organism the tissue was derived from or into another organism under conditions suitable to modulate the expression of the HBV gene in that organism.

In one embodiment, the invention features a method of modulating the expression of a HBV gene in a tissue explant, comprising: (a) synthesizing a siNA molecule of the invention, which can be chemically modified, wherein one of the siNA strands includes a sequence complementary to RNA of the HBV gene and wherein the sense strand sequence of the siNA is identical to the complementary sequence of the target RNA; (b) introducing the siNA molecule into a cell of the tissue explant derived from a particular organism under conditions suitable to modulate the expression of the HBV gene in the tissue explant, and (c) optionally introducing the tissue explant back into the organism the tissue was derived from or into another organism under conditions suitable to modulate the expression of the HBV gene in that organism.

In another embodiment, the invention features a method of modulating the expression of more than one HBV gene in a tissue explant, comprising: (a) synthesizing siNA molecules of the invention, which can be chemically modified, wherein one of the siNA strands includes a sequence complementary to RNA of the HBV genes; (b) introducing the siNA molecules into a cell of the tissue explant derived from a particular organism under conditions suitable to modulate the expression of the HBV genes in the tissue explant, and (c) optionally introducing the tissue explant back into the organism the tissue was derived from or into another organism under conditions suitable to modulate the expression of the HBV genes in that organism.

In one embodiment, the invention features a method of modulating the expression of a HBV gene in an organism, comprising: (a) synthesizing a siNA molecule of the invention, which can be chemically modified, wherein one of the siNA strands includes a sequence complementary to RNA of the HBV gene; and (b) introducing the siNA molecule into the organism under conditions suitable to modulate the expression of the HBV gene in the organism.

In another embodiment, the invention features a method of modulating the expression of more than one HBV gene in an organism, comprising: (a) synthesizing siNA molecules of the invention, which can be chemically modified, wherein one of the siNA strands includes a sequence complementary to RNA of the HBV genes; and (b) introducing the siNA molecules into the organism under conditions suitable to modulate the expression of the HBV genes in the organism.

The siNA molecules of the invention can be designed to inhibit target (HBV) gene expression through RNAi targeting of a variety of RNA molecules. In one embodiment, the siNA molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s), and/or RNA templates. If alternate splicing produces a family of transcipts that are distinguished by usage of appropriate exons, the instant invention can be used to inhibit gene expression through the appropriate exons to specifically inhibit or to distinguish among the functions of gene family members. For example, a protein that contains an alternatively spliced transmembrane domain can be expressed in both membrane bound and secreted forms. Use of the invention to target the exon containing the transmembrane domain can be used to determine the functional consequences of pharmaceutical targeting of membrane bound as opposed to the secreted form of the protein. Non-limiting examples of applications of the invention relating to targeting these RNA molecules include therapeutic pharmaceutical applications, pharmaceutical discovery applications, molecular diagnostic and gene function applications, and gene mapping, for example using single nucleotide polymorphism mapping with siNA molecules of the invention. Such applications can be implemented using known gene sequences or from partial sequences available from an expressed sequence tag (EST).

In another embodiment, the siNA molecules of the invention are used to target conserved sequences corresponding to a gene family or gene families such as HBV genes. As such, siNA molecules targeting multiple HBV targets can provide increased therapeutic effect. In addition, siNA can be used to characterize pathways of gene function in a variety of applications. For example, the present invention can be used to inhibit the activity of target gene(s) in a pathway to determine the function of uncharacterized gene(s) in gene function analysis, mRNA function analysis, or translational analysis. The invention can be used to determine potential target gene pathways involved in various diseases and conditions toward pharmaceutical development. The invention can be used to understand pathways of gene expression involved in, for example, HBV infection.

In one embodiment, siNA molecule(s) and/or methods of the invention are used to inhibit the expression of gene(s) that encode RNA referred to by Genbank Accession, for example HBV genes such genes encoding RNA sequence(s) referred to herein by Genbank Accession number, for example Genbank Accession No. AB073834 or Accession numbers shown in Table I. Such sequences are readily obtained using these Genbank Accession numbers.

In one embodiment, the invention features a method comprising: (a) generating a library of siNA constructs having a predetermined complexity; and (b) assaying the siNA constructs of (a) above, under conditions suitable to determine RNAi target sites within the target RNA sequence. In another embodiment, the siNA molecules of (a) have strands of a fixed length, for example about 23 nucleotides in length. In yet another embodiment, the siNA molecules of (a) are of differing length, for example having strands of about 19 to about 25 (e.g., about 19, 20, 21, 22, 23, 24, or 25) nucleotides in length. In yet another embodiment, the assay can comprise a reconstituted in vitro siNA assay as described herein. In another embodiment, the assay can comprise a cell culture system in which target RNA is expressed. In another embodiment, fragments of target RNA are analyzed for detectable levels of cleavage, for example by gel electrophoresis, northern blot analysis, or RNAse protection assays, to determine the most suitable target site(s) within the target target RNA sequence. In another embodiment, the target RNA sequence can be obtained as is known in the art, for example, by cloning and/or transcription for in vitro systems, and by cellular expression in in vivo systems.

In one embodiment, the invention features a method comprising: (a) generating a randomized library of siNA constructs having a predetermined complexity, such as of 4^{N}, where N represents the number of base paired nucleotides in each of the siNA construct strands (eg. for a siNA construct having 21-nucleotide sense and antisense strands with 19 base pairs, the complexity would be 4¹⁹); and (b) assaying the siNA constructs of (a) above, under conditions suitable to determine RNAi target sites within the target HBV RNA sequence. In another embodiment, the siNA molecules of (a) have strands of a fixed length, for example about 23 nucleotides in length. In yet another embodiment, the siNA molecules of (a) are of differing length, for example having strands of about 19 to about 25 (e.g., about 19, 20, 21, 22, 23, 24, or 25) nucleotides in length. In yet another embodiment, the assay can comprise a reconstituted in vitro siNA assay as described in Example 7 herein. In another embodiment, the assay can comprise a cell culture system in which target RNA is expressed. In another embodiment, fragments of HBV RNA are analyzed for detectable levels of cleavage, for example by gel electrophoresis, northern blot analysis, or RNAse protection assays, to determine the most suitable target site(s) within the target HBV RNA sequence. In another embodiment, the target HBV RNA sequence can be obtained as is known in the art, for example, by cloning and/or transcription for in vitro systems, and by cellular expression in in vivo systems.

In another embodiment, the invention features a method comprising: (a) analyzing the sequence of a RNA target encoded by a target gene; (b) synthesizing one or more sets of siNA molecules having sequence complementary to one or more regions of the RNA of (a); and (c) assaying the siNA molecules of (b) under conditions suitable to determine RNAi targets within the target RNA sequence. In another embodiment, the siNA molecules of (b) have strands of a fixed length, for example about 23 nucleotides in length. In yet another embodiment, the siNA molecules of (b) are of differing length, for example having strands of about 19 to about 25 (e.g., about 19, 20, 21, 22, 23, 24, or 25) nucleotides in length. In yet another embodiment, the assay can comprise a reconstituted in vitro siNA assay as described herein. In another embodiment, the assay can comprise a cell culture system in which target RNA is expressed. Fragments of target RNA are analyzed for detectable levels of cleavage, for example by gel electrophoresis, northern blot analysis, or RNAse protection assays, to determine the most suitable target site(s) within the target RNA sequence. The target RNA sequence can be obtained as is known in the art, for example, by cloning and/or transcription for in vitro systems, and by expression in in vivo systems.

By “target site” is meant a sequence within a target RNA that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

In one embodiment, the invention features a composition comprising a siNA molecule of the invention, which can be chemically modified, in a pharmaceutically acceptable carrier or diluent. In another embodiment, the invention features a pharmaceutical composition comprising siNA molecules of the invention, which can be chemically modified, targeting one or more genes in a pharmaceutically acceptable carrier or diluent. In another embodiment, the invention features a method for treating or preventing a disease or condition in a patient, comprising administering to the patient a composition of the invention under conditions suitable for the treatment or prevention of the disease or condition in the patient, alone or in conjunction with one or more other therapeutic compounds. In yet another embodiment, the invention features a method for reducing or preventing tissue rejection in a patient comprising administering to the patient a composition of the invention under conditions suitable for the reduction or prevention of tissue rejection in the patient.

In another embodiment, the invention features a method for validating a HBV gene target, comprising: (a) synthesizing a siNA molecule of the invention, which can be chemically modified, wherein one of the siNA strands includes a sequence complementary to RNA of a HBV target gene; (b) introducing the siNA molecule into a cell, tissue, or organism under conditions suitable for modulating expression of the HBV target gene in the cell, tissue, or organism; and (c) determining the function of the gene by assaying for any phenotypic change in the cell, tissue, or organism.

By “phenotypic change” is meant any detectable change to a cell that occurs in response to contact or treatment with a nucleic acid molecule of the invention (e.g., siNA). Such detectable changes include but are not limited to changes in shape, size, proliferation, protein expression or RNA expression or detection of viral antigens as can be assayed by methods known in the art. The detectable change can also include expression of reporter genes/molecules such as Green Florescent Protein (GFP) or various tags that are used to identify an expressed protein or any other cellular component that can be assayed.

In one embodiment, the invention features a kit containing a siNA molecule of the invention, which can be chemically modified, that can be used to modulate the expression of a HBV target gene in a cell, tissue, or organism. In another embodiment, the invention features a kit containing more than one siNA molecule of the invention, which can be chemically modified, that can be used to modulate the expression of more than one HBV target gene in a cell, tissue, or organism.

In one embodiment, the invention features a cell containing one or more siNA molecules of the invention, which can be chemically modified. In another embodiment, the cell containing a siNA molecule of the invention is a mammalian cell. In yet another embodiment, the cell containing a siNA molecule of the invention is a human cell.

In one embodiment, the synthesis of a siNA molecule of the invention, which can be chemically modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

In one embodiment, the invention features a method for synthesizing a siNA duplex molecule comprising: (a) synthesizing a first oligonucleotide sequence strand of the siNA molecule, wherein the first oligonucleotide sequence strand comprises a cleavable linker molecule that can be used as a scaffold for the synthesis of the second oligonucleotide sequence strand of the siNA; (b) synthesizing the second oligonucleotide sequence strand of siNA on the scaffold of the first oligonucleotide sequence strand, wherein the second oligonucleotide sequence strand further comprises a chemical moiety than can be used to purify the siNA duplex; (c) cleaving the linker molecule of (a) under conditions suitable for the two siNA oligonucleotide strands to hybridize and form a stable duplex; and (d) purifying the siNA duplex utilizing the chemical moiety of the second oligonucleotide sequence strand. In another embodiment, cleavage of the linker molecule in (c) above takes place during deprotection of the oligonucleotide, for example under hydrolysis conditions using an alkylamine base such as methylamine. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity as the solid support derivatized linker, such that cleavage of the solid support derivatized linker and the cleavable linker of (a) takes place concomitantly. In another embodiment, the chemical moiety of (b) that can used to isolate the attached oligonucleotide sequence comprises a trityl group, for example a dimethoxytrityl group, which can be employed in a trityl-on synthesis strategy as described herein. In yet another embodiment, the chemical moiety, such as a dimethoxytrityl group, is removed during purification, for example using acidic conditions.

In a further embodiment, the method for siNA synthesis is a solution phase synthesis or hybrid phase synthesis wherein both strands of the siNA duplex are synthesized in tandem using a cleavable linker attached to the first sequence which acts a scaffold for synthesis of the second sequence. Cleavage of the linker under conditions suitable for hybridization of the separate siNA sequence strands results in formation of the double-stranded siNA molecule.

In another embodiment, the invention features a method for synthesizing a siNA duplex molecule comprising: (a) synthesizing one oligonucleotide sequence strand of the siNA molecule, wherein the sequence comprises a cleavable linker molecule that can be used as a scaffold for the synthesis of another oligonucleotide sequence; (b) synthesizing a second oligonucleotide sequence having complementarity to the first sequence strand on the scaffold of (a), wherein the second sequence comprises the other strand of the double-stranded siNA molecule and wherein the second sequence further comprises a chemical moiety than can be used to isolate the attached oligonucleotide sequence; (c) purifying the product of (b) utilizing the chemical moiety of the second oligonucleotide sequence strand under conditions suitable for isolating the full-length sequence comprising both siNA oligonucleotide strands connected by the cleavable linker; and (d) under conditions suitable for the two siNA oligonucleotide strands to hybridize and form a stable duplex. In another embodiment, cleavage of the linker molecule in (c) above takes place during deprotection of the oligonucleotide, for example under hydrolysis conditions. In another embodiment, cleavage of the linker molecule in (c) above takes place after deprotection of the oligonucleotide. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity or differing reactivity as the solid support derivatized linker, such that cleavage of the solid support derivatized linker and the cleavable linker of (a) takes place either concomitantly or sequentially. In another embodiment, the chemical moiety of (b) that can used to isolate the attached oligonucleotide sequence comprises a trityl group, for example a dimethoxytrityl group.

In another embodiment, the invention features a method for making a double-stranded siNA molecule in a single synthetic process, comprising: (a) synthesizing an oligonucleotide having a first and a second sequence, wherein the first sequence is complementary to the second sequence, and the first oligonucleotide sequence is linked to the second sequence via a cleavable linker, and wherein a terminal 5′-protecting group, for example a 5′-O-dimethoxytrityl group (5′-O-DMT) remains on the oligonucleotide having the second sequence; (b) deprotecting the oligonucleotide whereby the deprotection results in the cleavage of the linker joining the two oligonucleotide sequences; and (c) purifying the product of (b) under conditions suitable for isolating the double-stranded siNA molecule, for example using a trityl-on synthesis strategy as described herein.

In one embodiment, the invention features siNA constructs that mediate RNAi against HBV, wherein the siNA construct comprises one or more chemical modifications, for example one or more chemical modifications having any of Formulae I-VII or any combination thereof that increases the nuclease resistance of the siNA construct.

In another embodiment, the invention features a method for generating siNA molecules with increased nuclease resistance comprising (a) introducing nucleotides having any of Formula I-VII into a siNA molecule, and (b) assaying the siNA molecule of step (a) under conditions suitable for isolating siNA molecules having increased nuclease resistance.

In one embodiment, the invention features siNA constructs that mediate RNAi against HBV, wherein the siNA construct comprises one or more chemical modifications described herein that modulates the binding affinity between the sense and antisense strands of the siNA construct.

In another embodiment, the invention features a method for generating siNA molecules with increased binding affinity between the sense and antisense strands of the siNA molecule comprising (a) introducing nucleotides having any of Formula I-VII into a siNA molecule, and (b) assaying the siNA molecule of step (a) under conditions suitable for isolating siNA molecules having increased binding affinity between the sense and antisense strands of the siNA molecule.

In one embodiment, the invention features siNA constructs that mediate RNAi against HBV, wherein the siNA construct comprises one or more chemical modifications described herein that modulates the binding affinity between the antisense strand of the siNA construct and a complementary target RNA sequence within a cell.

In another embodiment, the invention features a method for generating siNA molecules with increased binding affinity between the antisense strand of the siNA molecule and a complementary target RNA sequence, comprising (a) introducing nucleotides having any of Formula I-VII into a siNA molecule, and (b) assaying the siNA molecule of step (a) under conditions suitable for isolating siNA molecules having increased binding affinity between the antisense strand of the siNA molecule and a complementary target RNA sequence.

In one embodiment, the invention features siNA constructs that mediate RNAi against HBV, wherein the siNA construct comprises one or more chemical modifications described herein that modulate the polymerase activity of a cellular polymerase capable of generating additional endogenous siNA molecules having sequence homology to the chemically-modified siNA construct.

In another embodiment, the invention features a method for generating siNA molecules capable of mediating increased polymerase activity of a cellular polymerase capable of generating additional endogenous siNA molecules having sequence homology to the chemically-modified siNA molecule comprising (a) introducing nucleotides having any of Formula I-VII into a siNA molecule, and (b) assaying the siNA molecule of step (a) under conditions suitable for isolating siNA molecules capable of mediating increased polymerase activity of a cellular polymerase capable of generating additional endogenous siNA molecules having sequence homology to the chemically-modified siNA molecule.

In one embodiment, the invention features chemically-modified siNA constructs that mediate RNAi against HBV in a cell, wherein the chemical modifications do not significantly effect the interaction of siNA with a target RNA molecule and/or proteins or other factors that are essential for RNAi in a manner that would decrease the efficacy of RNAi mediated by such siNA constructs.

In another embodiment, the invention features a method for generating siNA molecules with improved RNAi activity against HBV, comprising (a) introducing nucleotides having any of Formula I-VII into a siNA molecule, and (b) assaying the siNA molecule of step (a) under conditions suitable for isolating siNA molecules having improved RNAi activity.

In yet another embodiment, the invention features a method for generating siNA molecules with improved RNAi activity against an HBV target RNA, comprising (a) introducing nucleotides having any of Formula I-VII into a siNA molecule, and (b) assaying the siNA molecule of step (a) under conditions suitable for isolating siNA molecules having improved RNAi activity against the target RNA.

In one embodiment, the invention features siNA constructs that mediate RNAi against HBV, wherein the siNA construct comprises one or more chemical modifications described herein that modulates the cellular uptake of the siNA construct.

In another embodiment, the invention features a method for generating siNA molecules against HBV with improved cellular uptake, comprising (a) introducing nucleotides having any of Formula I-VII into a siNA molecule, and (b) assaying the siNA molecule of step (a) under conditions suitable for isolating siNA molecules having improved cellular uptake.

In one embodiment, the invention features siNA constructs that mediate RNAi against HBV, wherein the siNA construct comprises one or more chemical modifications described herein that increases the bioavailability of the siNA construct, for example by attaching polymeric conjugates such as polyethyleneglycol or equivalent conjugates that improve the pharmacokinetics of the siNA construct, or by attaching conjugates that target specific tissue types or cell types in vivo. Non-limiting examples of such conjugates are described in Vargeese et al., U.S. Ser. No. 60/311,865 incorporated by reference herein.

In one embodiment, the invention features a method for generating siNA molecules of the invention with improved bioavailability, comprising (a) introducing a conjugate into the structure of a siNA molecule, and (b) assaying the siNA molecule of step (a) under conditions suitable for isolating siNA molecules having improved bioavailability. Such conjugates can include ligands for cellular receptors such as peptides derived from naturally occurring protein ligands, protein localization sequences including cellular ZIP code sequences, antibodies, nucleic acid aptamers, vitamins and other co-factors such as folate and N-acetylgalactosamine, polymers such as polyethyleneglycol (PEG), phospholipids, polyamines such as spermine or spermidine, and others.

In another embodiment, the invention features a method for generating siNA molecules of the invention with improved bioavailability, comprising (a) introducing an excipient formulation to a siNA molecule, and (b) assaying the siNA molecule of step (a) under conditions suitable for isolating siNA molecules having improved bioavailability. Such excipients include polymers such as cyclodextrines, lipids, cationic lipids, polyamines, phospholipids, and others.

In another embodiment, the invention features a method for generating siNA molecules of the invention with improved bioavailability, comprising (a) introducing nucleotides having any of Formula I-VII into a siNA molecule, and (b) assaying the siNA molecule of step (a) under conditions suitable for isolating siNA molecules having improved bioavailability.

In another embodiment, polyethylene glycol (PEG) can be covalently attached to siNA compounds of the present invention. The attached PEG can be any molecular weight, preferably from about 2,000 to about 50,000 daltons (Da).

The present invention can be used alone or as a component of a kit having at least one of the reagents necessary to carry out the in vitro or in vivo introduction of RNA to test samples and/or subjects. For example, preferred components of the kit include the siNA and a vehicle that promotes introduction of the siNA. Such a kit can also include instructions to allow a user of the kit to practice the invention.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference (“RNAi”) or gene silencing; see for example Bass, 2001, Nature, 411, 428-429; Elbashir et al., 2001, Nature, 411, 494-498; Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. Non-limiting examples of siRNA molecules of the invention are shown in FIG. 10. For example the siNA can be a double-stranded polynucleotide molecule comprising self-complementary sense and antisense regions, wherein the antisense region comprises complementarity to a target nucleic acid molecule. The siNA can be a single-stranded hairpin polynucleotide having self-complementary sense and antisense regions, wherein the antisense region comprises complementarity to a target nucleic acid molecule. The siNA can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active siNA capable of mediating RNAi. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not contain any ribonucleotides (e.g., nucleotides having a 2′-OH group). The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA, short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post-transciptional gene silencing.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit” but the use of the word “modulate” is not limited to this definition.

By “inhibit” it is meant that the activity of a gene expression product or level of RNAs or equivalent RNAs encoding one or more gene products is reduced below that observed in the absence of the nucleic acid molecule of the invention. In one embodiment, inhibition with a siNA molecule preferably is below that level observed in the presence of an inactive or attenuated molecule that is unable to mediate an RNAi response. In another embodiment, inhibition of gene expression with the siNA molecule of the instant invention is greater in the presence of the siNA molecule than in its absence.

By “gene” or “target gene” is meant, a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

By “HBV proteins” is meant, a peptide or protein comprising a component of HBV and/or encoded by a HBV gene.

By “highly conserved sequence region” is meant, a nucleotide sequence of one or more regions in a target gene does not vary significantly from one generation to the other or from one biological system to the other.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The siNA molecules of the invention represent a novel therapeutic approach to treat a variety of pathologic indications, such as HBV infection, liver failure, cirrhosis, hepatocellular carcinoma, and any other diseases or conditions that are related to or will respond to the levels of HBV in a cell or tissue, alone or in combination with other therapies. The reduction of HBV expression (specifically HBV gene RNA levels) and thus reduction in the level of the respective protein relieves, to some extent, the symptoms of the disease or condition.

In one embodiment of the present invention, each sequence of a siNA molecule of the invention is independently 18 to 24 nucleotides in length, in specific embodiments about 18, 19, 20, 21, 22, 23, or 24 nucleotides in length. In another embodiment, the siNA duplexes of the invention independently comprise between 17 and 23 base pairs. In yet another embodiment, siNA molecules of the invention comprising hairpin or circular structures are 35 to 55 nucleotides in length, or 38-44 nucleotides in length and comprising 16-22 base pairs. Exemplary siNA molecules of the invention are shown in Table II. Exemplary synthetic siNA molecules of the invention are shown in Table III and/or FIGS. 4-5.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

The siNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Tables II-III and/or FIGS. 4-5. Examples of such nucleic acid molecules consist essentially of sequences defined in these tables and figures.

In another aspect, the invention provides mammalian cells containing one or more siNA molecules of this invention. The one or more siNA molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The term “phosphorothioate” as used herein refers to an internucleotide linkage having Formula I, wherein Z and/or W comprise a sulfur atom. Hence, the term phosphorothioate refers to both phosphorothioate and phosphorodithioate internucleotide linkages.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example where any of the ribose carbons (C1, C2, C3, C4, or C5), are independently or in combination absent from the nucleotide.

The nucleic acid molecules of the instant invention, individually, or in combination or in conjunction with other drugs, can be used to treat diseases or conditions discussed herein. For example, to treat a particular disease or condition, the siNA molecules can be administered to a patient or can be administered to other appropriate cells evident to those skilled in the art, individually or in combination with one or more drugs under conditions suitable for the treatment.

In a further embodiment, the siNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In one embodiment, the invention features an expression vector comprising a nucleic acid sequence encoding at least one siNA molecule of the invention, in a manner that allows expression of the siNA molecule. For example, the vector can contain sequence(s) encoding both strands of a siNA molecule comprising a duplex. The vector can also contain sequence(s) encoding a single nucleic acid molecule that is self-complementary and thus forms a siNA molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725.

In another embodiment, the invention features a mammalian cell, for example, a human cell, including an expression vector of the invention.

In yet another embodiment, the expression vector of the invention comprises a sequence for a siNA molecule having complementarity to a RNA molecule referred to by a Genbank Accession number, for example Genbank Accession No. AB073834 or Genbank Accession Nos. shown in Table I.

In one embodiment, an expression vector of the invention comprises a nucleic acid sequence encoding two or more siNA molecules, which can be the same or different.

In another aspect of the invention, siNA molecules that interact with target RNA molecules and down-regulate gene encoding target RNA molecules (for example target RNA molecules referred to by Genbank Accession numbers herein) are expressed from transcription units inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules. Such vectors can be repeatedly administered as necessary. Once expressed, the siNA molecules bind and down-regulate gene function or expression via RNA interference (RNAi). Delivery of siNA expressing vectors can be systemic, such as by intravenous or intramuscular administration, by administration to target cells ex-planted from a patient followed by reintroduction into the patient, or by any other means that would allow for introduction into the desired target cell.

By “vectors” is meant any nucleic acid- and/or viral-based technique used to deliver a desired nucleic acid.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

Drawings

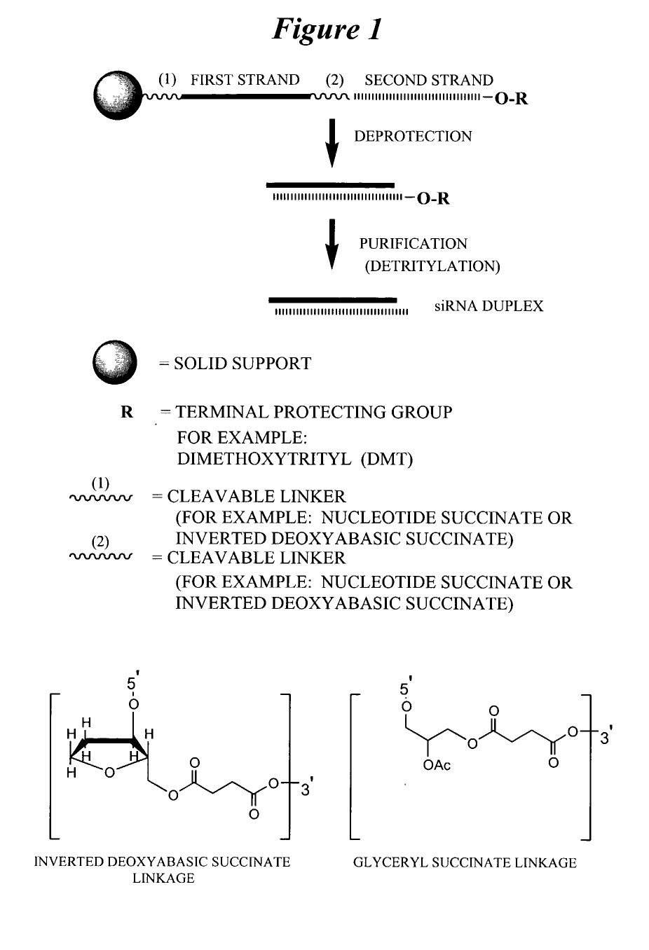

FIG. 1 shows a non-limiting example of a scheme for the synthesis of siNA molecules. The complementary siNA sequence strands, strand 1 and strand 2, are synthesized in tandem and are connected by a cleavable linkage, such as a nucleotide succinate or abasic succinate, which can be the same or different from the cleavable linker used for solid phase synthesis on a solid support. The synthesis can be either solid phase or solution phase, in the example shown, the synthesis is a solid phase synthesis. The synthesis is performed such that a protecting group, such as a dimethoxytrityl group, remains intact on the terminal nucleotide of the tandem oligonucleotide. Upon cleavage and deprotection of the oligonucleotide, the two siNA strands spontaneously hybridize to form a siNA duplex, which allows the purification of the duplex by utilizing the properties of the terminal protecting group, for example by applying a trityl on purification method wherein only duplexes/oligonucleotides with the terminal protecting group are isolated.

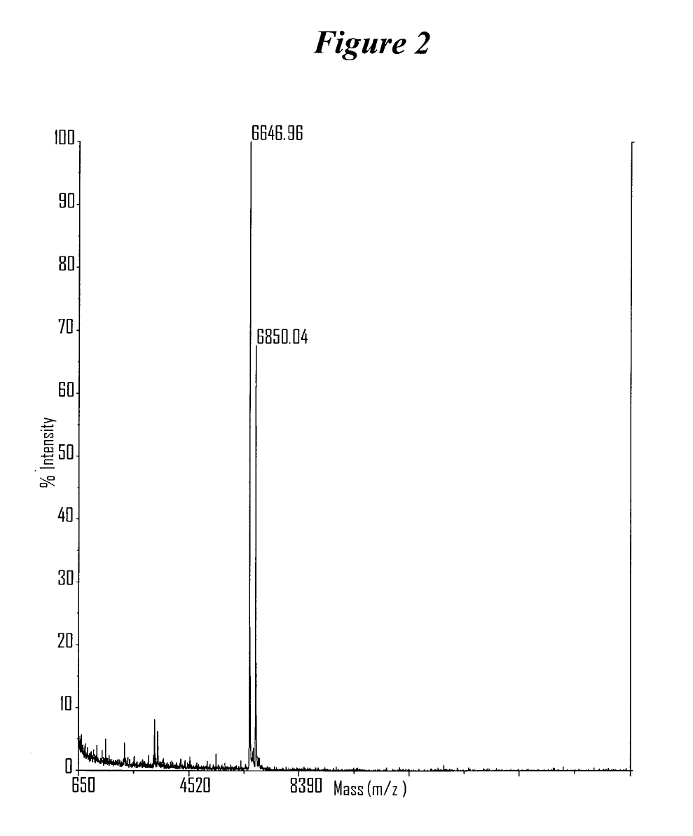

FIG. 2 shows a MALDI-TOV mass spectrum of a purified siNA duplex synthesized by a method of the invention. The two peaks shown correspond to the predicted mass of the separate siNA sequence strands. This result demonstrates that the siNA duplex generated from tandem synthesis can be purified as a single entity using a simple trityl-on purification methodology.

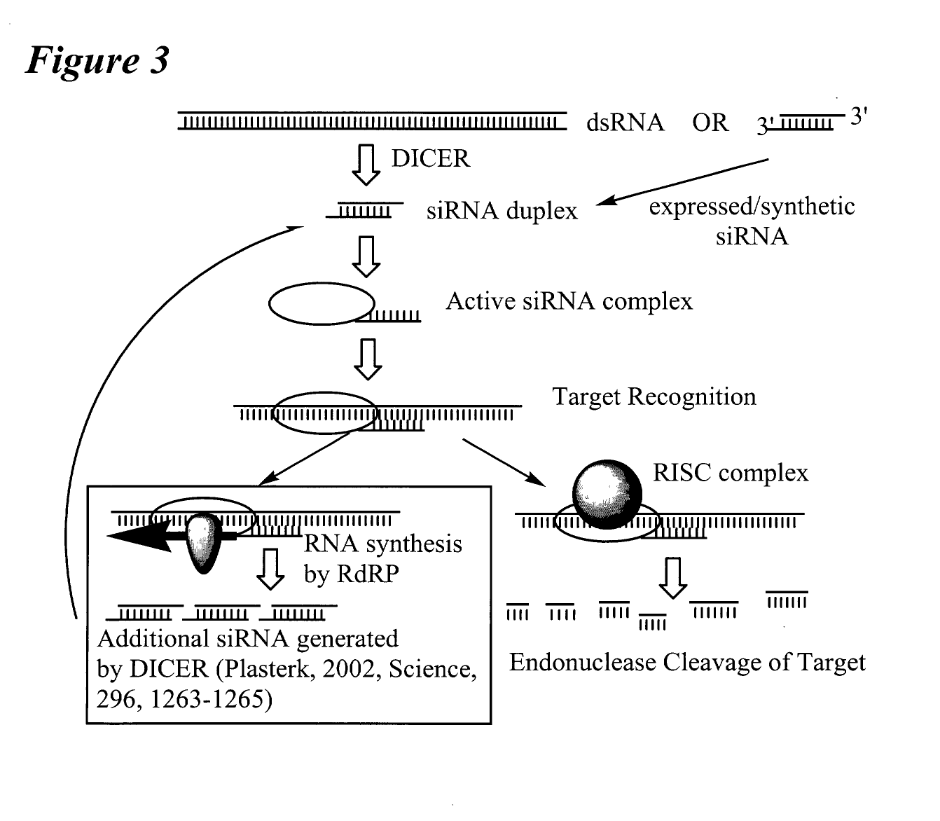

FIG. 3 shows a non-limiting proposed mechanistic representation of target RNA degradation involved in RNAi. Double stranded RNA (dsRNA), which is generated by RNA-dependent RNA polymerase (RdRP) from foreign single-stranded RNA, for example viral, transposon, or other exogenous RNA, activates the DICER enzyme that in turn generates siNA duplexes. Alternately, synthetic or expressed siNA can be introduced directely into a cell by appropriate means. An active siNA complex forms which recognizes a target RNA, resulting in degradation of the target RNA by the RISC endonuclease complex or in the synthesis of additional RNA by RNA-dependent RNA polymerase (RdRP), which can activate DICER and result in additional siNA molecules, thereby amplifying the RNAi response.

FIGS. 4A-F shows non-limiting examples of chemically-modified siNA constructs of the present invention. In the figure, N stands for any nucleotide (adenosine, guanosine, cytosine, uridine, or optionally thymidine, for example thymidine can be substituted in the overhanging regions designated by parenthesis (N N). Various modifications are shown for the sense and antisense strands of the siNA constructs.

FIG. 4A: The sense strand comprises 21 nucleotides having four phosphorothioate 5′ and 3′-terminal internucleotide linkages, wherein the two terminal 3′-nucleotides are optionally base paired and wherein all pyrimidine nucleotides that may be present are 2′-O-methy or 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-tenninal glyceryl moiety and wherein the two terminal 3′-nucleotides are optionally complementary to the target RNA sequence, and having one 3′-terminal phosphorothioate internucleotide linkage and four 5′-terminal phosphorothioate internucleotide linkages and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein.

FIG. 4B: The sense strand comprises 21 nucleotides wherein the two terminal 3′-nucleotides are optionally base paired and wherein all pyrimidine nucleotides that may be present are 2′-O-methyl or 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein.

FIG. 4C: The sense strand comprises 21 nucleotides having 5′- and 3′-terminal cap moieties wherein the two terminal 3′-nucleotides are optionally base paired and wherein all pyrimidine nucleotides that may be present are 2′-O-methyl or 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and having one 3′-terminal phosphorothioate internucleotide linkage and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein.

FIG. 4D: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein and wherein and all purine nucleotides that may be present are 2′-deoxy nucleotides. The antisense strand comprises 21 nucleotides, optionally having a 3′-terminal glyceryl moiety and wherein the two terminal 3′-nucleotides are optionally complementary to the target RNA sequence, and having one 3′-terminal phosphorothioate internucleotide linkage and wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein.

FIG. 4E: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein.

FIG. 4F: The sense strand comprises 21 nucleotides having 5′- and 3′-terminal cap moieties wherein the two terminal 3′-nucleotides are optionally base paired and wherein all pyrimidine nucleotides that may be present are 2′-deoxy-2′-fluoro modified nucleotides except for (N N) nucleotides, which can comprise ribonucleotides, deoxynucleotides, universal bases, or other chemical modifications described herein. The antisense strand comprises 21 nucleotides, optionally having a 3′-terminal glyceryl moiety and wherein the two terminal 3′-nucleotides are optionally complementary to the target RNA sequence, and having one 3′-terminal phosphorothioate internucleotide linkage and wherein all pyrimidine nucleotides that may be present are 2′-deoxy-2′-fluoro modified nucleotides and all purine nucleotides that may be present are 2′-O-methyl modified nucleotides except for (N N) nucleotides, which can comprise ribonucleotides, deoxynucleotides, universal bases, or other chemical modifications described herein. The antisense strand of constructs A-F comprise sequence complementary to target RNA sequence of the invention.

FIGS. 5A-F shows non-limiting examples of specific chemically-modified siNA sequences of the invention. FIGS. 5A-F applies the chemical modifications described in FIGS. 4A-F to an HBV siNA sequence.

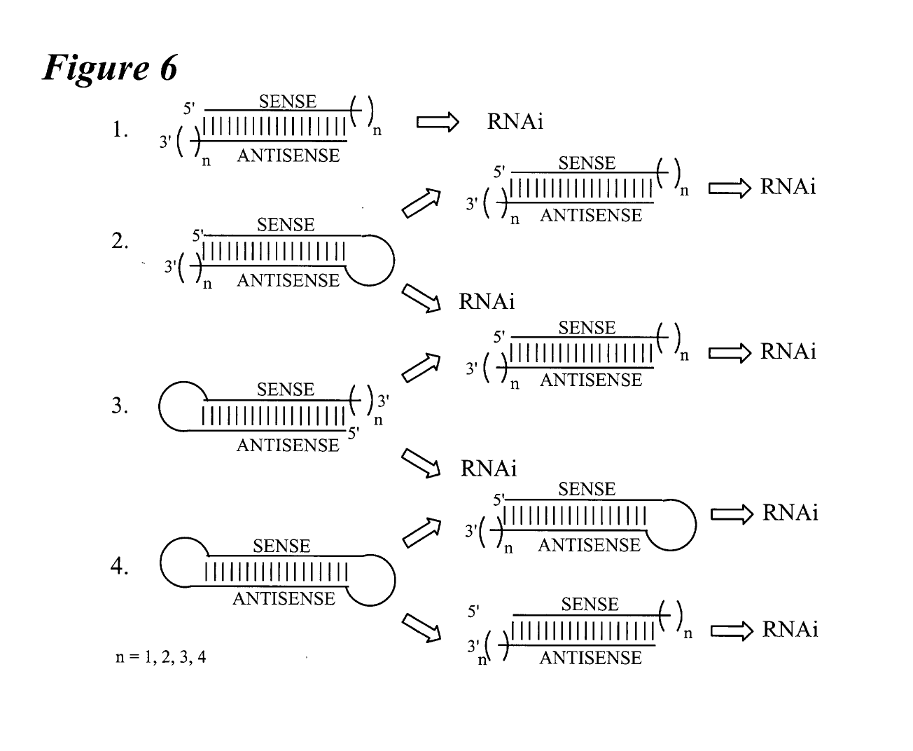

FIG. 6 shows non-limiting examples of different siNA constructs of the invention. The examples shown (constructs 1, 2, and 3) have 19 representative base pairs; however, different embodiments of the invention include any number of base pairs described herein. Bracketed regions represent nucleotide overhangs, for example comprising between about 1, 2, 3, or 4 nucleotides in length, preferably about 2 nucleotides. Constructs 1 and 2 can be used independently for RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siNA construct 2 in vivo and/or in vitro, which can optionally utilize another biodegradable linker to generate the active siNA construct 1 in vivo and/or in vitro. As such, the stability and/or activity of the siNA constructs can be modulated based on the design of the siNA construct for use in vivo or in vitro and/or in vitro.

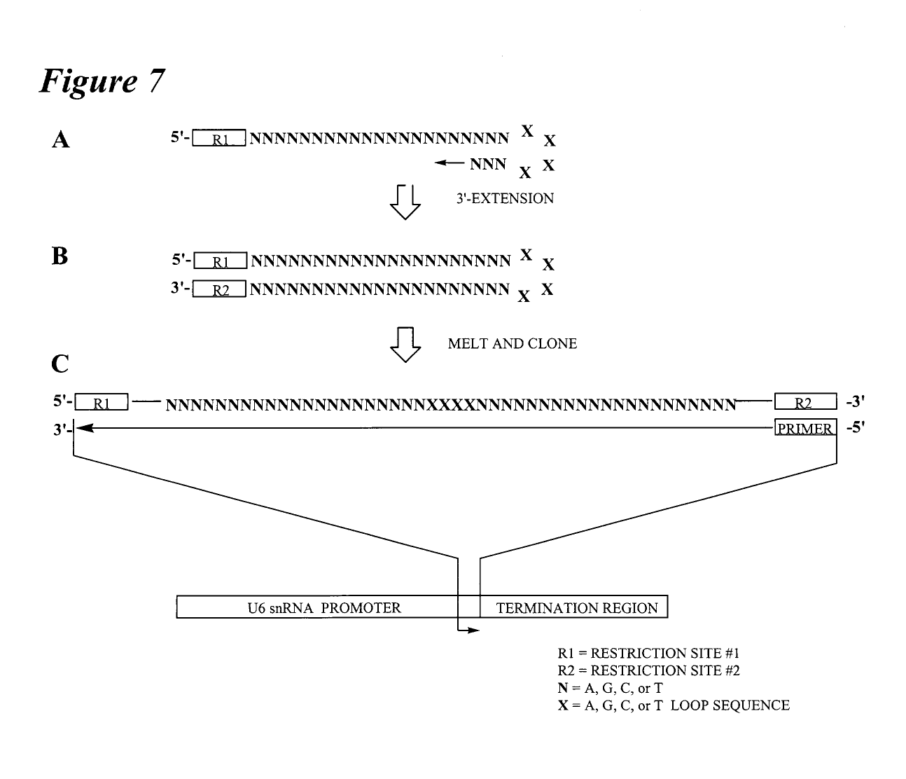

FIGS. 7A-C is a diagrammatic representation of a scheme utilized in generating an expression cassette to generate siNA hairpin constructs.

FIG. 7A: A DNA oligomer is synthesized with a 5′-restriction site (R1) sequence followed by a region having sequence identical (sense region of siNA) to a predetermined HBV target seqeunce, wherein the sense region comprises, for example, about 19, 20, 21, or 22 nucleotides (N) in length, which is followed by a loop sequence of defined sequence (X), comprising, for example, between about 3 and 10 nucleotides.

FIG. 7B: The synthetic construct is then extended by DNA polymerase to generate a hairpin structure having self-complementary sequence that will result in a siNA transcript having specificity for an HBV target sequence and having self-complementary sense and antisense regions.

FIG. 7C: The construct is heated (for example to about 95° C.) to linearize the sequence, thus allowing extension of a complementary second DNA strand using a primer to the 3′-restriction sequence of the first strand. The double-stranded DNA is then inserted into an appropriate vector for expression in cells. The construct can be designed such that a 3′-terminal nucleotide overhang results from the transcription, for example by engineering restriction sites and/or utilizing a poly-U termination region as described in Paul et al., 2002, Nature Biotechnology, 29, 505-508.

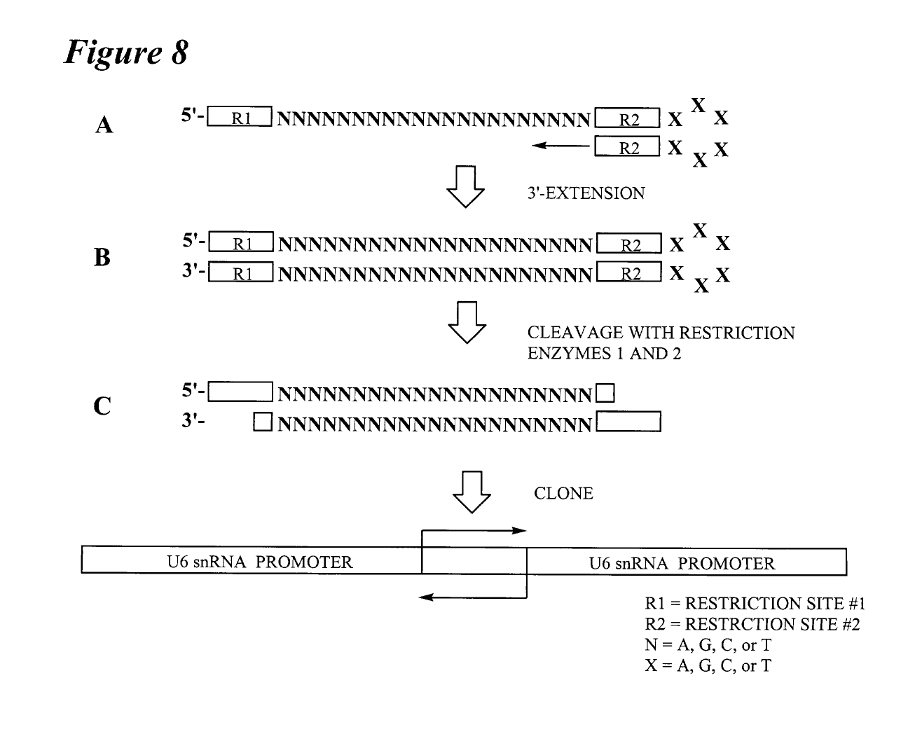

FIGS. 8A-C is a diagrammatic representation of a scheme utilized in generating an expression cassette to generate double-stranded siNA constructs.

FIG. 8A: A DNA oligomer is synthesized with a 5′-restriction (R1) site sequence followed by a region having sequence identical (sense region of siNA) to a predetermined HBV target seqeunce, wherein the sense region comprises, for example, about 19, 20, 21, or 22 nucleotides (N) in length, and which is followed by a 3′-restriction site (R2) which is adjacent to a loop sequence of defined sequence (X).

FIG. 8B: The synthetic construct is then extended by DNA polymerase to generate a hairpin structure having self-complementary sequence.

FIG. 8C: The construct is processed by restriction enzymes specific to R1 and R2 to generate a double-stranded DNA which is then inserted into an appropriate vector for expression in cells. The transcription cassette is designed such that a U6 promoter region flanks each side of the dsDNA which generates the separate sense and antisense strands of the siNA. Poly T termination sequences can be added to the constructs to generate U overhangs in the resulting transcript.

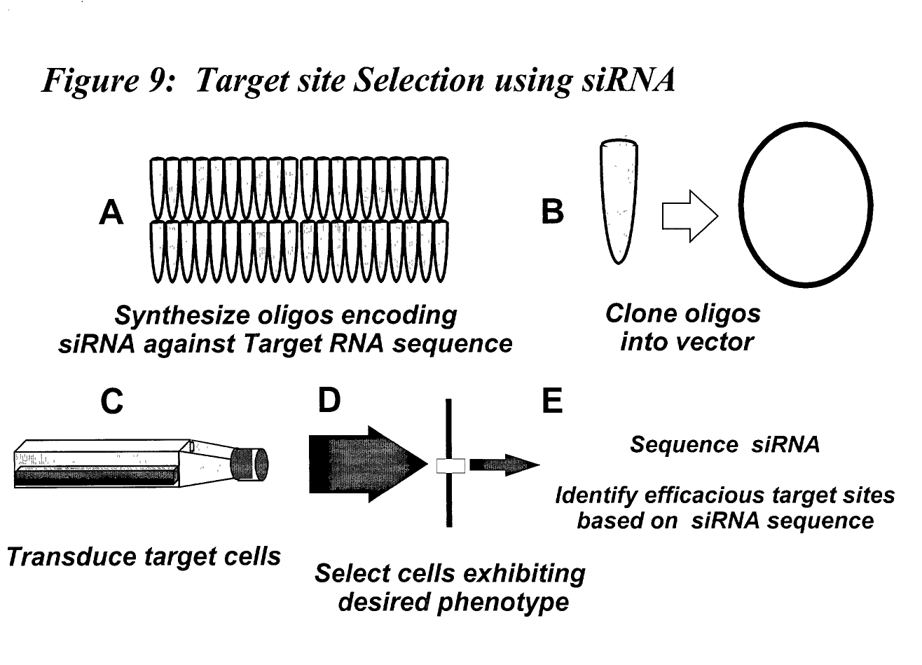

FIGS. 9A-E is a diagrammatic representation of a method used to determine target sites for siNA mediated RNAi within a particular target nucleic acid sequence, such as messenger RNA.

FIG. 9A: A pool of siNA oligonucleotides are synthesized wherein the antisense region of the siNA constructs has complementarity to target sites across the target nucleic acid sequence, and wherein the sense region comprises sequence complementary to the antisense region of the siNA.

FIGS. 9B & C: (FIG. 9B) The sequences are pooled and are inserted into vectors such that (FIG. 9C) transfection of a vector into cells results in the expression of the siNA.

FIG. 9D: Cells are sorted based on phenotypic change that is associated with modulation of the target nucleic acid sequence.

FIG. 9E The siNA is isolated from the sorted cells and is sequenced to identify efficacious target sites within the target nucleic acid sequence.

FIG. 10 shows non-limiting examples of different stabilization chemistries (1-10) that can be used, for example, to stabilize the 3′-end of siNA sequences of the invention, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3-5′]-3′-deoxyribonucleotide; (8) [3′-3′]-deoxyribonucleotide; (9) [5′-2′]-deoxyribonucleotide; and (10) [5-3′]-dideoxyribonucleotide. In addition to modified and unmodified backbone chemistries indicated in the figure, these chemistries can be combined with different backbone modifications as described herein, for example, backbone modifications having Formula I. In addition, the 2′-deoxy nucleotide shown 5′ to the terminal modifications shown can be another modified or unmodified nucleotide or non-nucleotide described herein, for example modifications having Formulae I-VII.

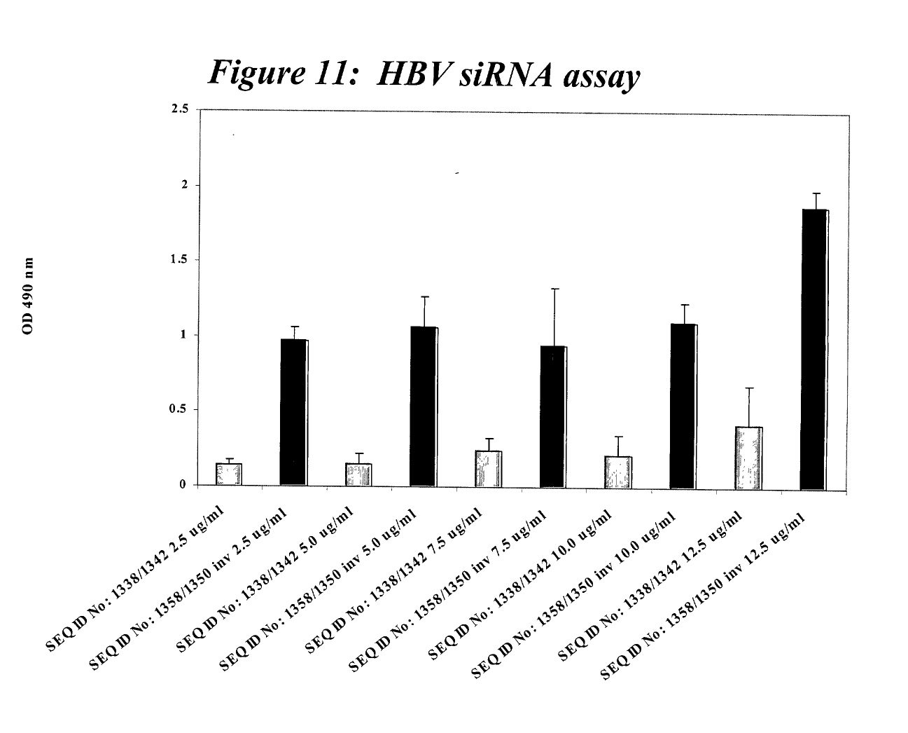

FIG. 11 shows a graphical representation of siNA mediated inhibition of HBV in a cell culture experiment. Results are shown with reference to the siRNA construct used (sense strand SEQ ID NO: 1338/antisense strand SEQ ID NO: 1342) at different lipid concentrations (2.5, 5.0, 7.5, 10.0 and 12.5 ug/ml). Inverted sequence duplexes were used as negative controls (sense strand SEQ ID NO: 1358/antisense strand SEQ ID NO: 1350). Levels of secreted HBV surface antigen (HBsAg) were analyzed by ELISA.

### MECHANISM OF ACTION OF NUCLEIC ACID MOLECULES OF THE INVENTION

The discussion that follows discusses the proposed mechanism of RNA interference mediated by short interfering RNA as is presently known, and is not meant to be limiting and is not an admission of prior art. Applicant demonstrates herein that chemically-modified short interfering nucleic acids possess similar or improved capacity to mediate RNAi as do siRNA molecules and are expected to possess improved stability and activity in vivo; therefore, this discussion is not meant to be limiting only to siRNA and can be applied to siNA as a whole. By “improved capacity to mediate RNAi” is meant to include RNAi activity measured in vitro and/or in vivo where the RNAi activity is a reflection of both the ability of the siNA to mediate RNAi and the stability of the siRNAs of the invention. In this invention, the product of these activities can be increased in vitro and/or in vivo compared to an all RNA siRNA or an siNA containing a plurality of ribonucleotides. In some cases, the activity or stability of the siNA molecule can be decreased (i.e., less than ten-fold), but the overall activity of the siNA molecule is enhanced, in vitro and/or in vivo RNA interference refers to the process of sequence specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double-stranded RNAs (dsRNAs) derived from viral infection or the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′, 5′-oligoadenylate synthetase resulting in non-specific cleavage of mRNA by ribonuclease L.

The presence of long dsRNAs in cells stimulates the activity of a ribonuclease III enzyme referred to as Dicer. Dicer is involved in the processing of the dsRNA into short pieces of dsRNA known as short interfering RNAs (siRNAs) (Berstein et al., 2001, Nature, 409, 363). Short interfering RNAs derived from Dicer activity are typically about 21-23 nucleotides in length and comprise about 19 base pair duplexes. Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21-nucleotide siRNA duplexes are most active when containing two 2-nucleotide 3′-terminal nucleotide overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309); however siRNA molecules lacking a 5′-phosphate are active when introduced exogenously, suggesting that 5′-phosphorylation of siRNA constructs may occur in vivo.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides or 2′-deoxy-2′-fluoro nucleotides. Table IV outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA including certain siNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table IV outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format.

Alternatively, the nucleic acid molecules of the present invention can be synthesized separately and joined together post-synthetically, for example, by ligation (Moore et al., 1992, Science 256, 9923; Draper et al., International PCT Publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siNA molecules of the invention can also be synthesized via a tandem synthesis methodology as described in Example 1 herein, wherein both siNA strands are synthesized as a single contiguous oligonucleotide fragment or strand separated by a cleavable linker which is subsequently cleaved to provide separate siNA fragments or strands that hybridize and permit purification of the siNA duplex. The linker can be a polynucleotide linker or a non-nucleotide linker. The tandem synthesis of siNA as described herein can be readily adapted to both multiwell/multiplate synthesis platforms such as 96 well or similarly larger multi-well platforms. The tandem synthesis of siNA as described herein can also be readily adapted to large scale synthesis platforms employing batch reactors, synthesis columns and the like.

A siNA molecule can also be assembled from two distinct nucleic acid strands or fragments wherein one fragment includes the sense region and the second fragment includes the antisense region of the RNA molecule.

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). siNA constructs can be purified by gel electrophoresis using general methods or can be purified by high pressure liquid chromatography (HPLC; see Wincott et al., supra, the totality of which is hereby incorporated herein by reference) and re-suspended in water.

In another aspect of the invention, siNA molecules of the invention are expressed from transcription units inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules.

Optimizing Activity of the Nucleic Aacid Molecule of the Invention

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, and/or 2′-H nucleotide base modifications (for a review, see Usman and Cedergren, 1992, TIBS, 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication, PCT No. WO 92/07065; Perrault et al. Nature, 1990, 344, 565-568; Picken et al., Science, 1991, 253, 314-317; Usman and Cedergren, Trends in Biochem. Sci., 1992, 17, 334-339; Usman et al., International Publication PCT No. WO 93/15187; Sproat, U.S. Pat. No. 5,334,711; Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 (filed on Apr. 20, 1998); Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify the siNA nucleic acid molecules of the instant invention so long as the ability of siNA to promote RNAi is cells is not significantly inhibited.

While chemical modification of oligonucleotide internucleotide linkages with phosphorothioate, phosphorothioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

Short interfering nucleic acid (siNA) molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995, Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g., about 1, 2, 3, 4, 5, 6, 7, 8, 9, 10, or more) LNA “locked nucleic acid” nucleotides such as a 2′, 4′-C mythylene bicyclo nucleotide (see for example Wengel et al., International PCT Publication No. WO 00/66604 and WO 99/14226).

In another embodiment, the invention features conjugates and/or complexes of siNA molecules of the invention. Such conjugates and/or complexes can be used to facilitate delivery of siNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable linker” as used herein, refers to a nucleic acid or non-nucleic acid linker molecule that is designed as a biodegradable linker to connect one molecule to another molecule, for example, a biologically active molecule to a siNA molecule of the invention or the sense and antisense strands of a siNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base-modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with othe molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., siNA molecules) delivered exogenously optimally are stable within cells until reverse trascription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, siNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo the activity should not be significantly lowered.

Use of the nucleic acid-based molecules of the invention will lead to better treatment of the disease progression by affording the possibility of combination therapies (e.g., multiple siNA molecules targeted to different genes; nucleic acid molecules coupled with known small molecule modulators; or intermittent treatment with combinations of molecules, including different motifs and/or other chemical or biological molecules). The treatment of subjects with siNA molecules can also include combinations of different types of nucleic acid molecules, such as enzymatic nucleic acid molecules (ribozymes), allozymes, antisense molecules, 2,5-A oligoadenylate, decoys, and aptamers.

In another aspect a siNA molecule of the invention comprises one or more 5′- and/or a 3′-cap structure, for example on only the sense siNA strand, antisense siNA strand, or both siNA strands.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples: the 5′-cap is selected from the group comprising glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In yet another embodiment, the 3′-cap is selected from a group comprising glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine, and therefore lacks a base at the 1′-position.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of β-D-ribo-furanose.

By “modified nucleoside” is meant any nucleotide base that contains a modification in the chemical structure of an unmodified nucleotide base, sugar and/or phosphate.

In connection with 2′-modified nucleotides as described for the present invention, by “amino” is meant 2′-NH₂ or 2′-O- NH₂, which may be modified or unmodified. Such modified groups are described, for example, in Eckstein et al., U.S. Pat. No. 5,672,695 and Matulic-Adamic et al., U.S. Pat. No. 6,248,878, which are both incorporated by reference in their entireties.

Various modifications to nucleic acid siNA structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

A siNA molecule of the invention can be adapted for use to treat, for example, HBV infection, liver failure cirrhosis, hepatocellular carcinoma and any other indications that can respond to the level of HBV in a cell or tissue, alone or in combination with other therapies. For example, a siNA molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713, and Sullivan et al., PCT WO 94/02595, further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al., 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication canincrease the beneficial effects while reducing the presence of side effects.

In one embodiment, the invention comprises compositions suitable for administering nucleic acid molecules of the invention to specific cell types, such as hepatocytes. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain siNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990, Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the siNA molecules can be delivered as described above, and persist in target cells. Alternatively, viral vectors can be used that provide for transient expression of nucleic acid molecules. Such vectors can be repeatedly administered as necessary. Once expressed, the siNA molecule interacts with the target mRNA and generates an RNAi response. Delivery of siNA molecule expressing vectors can be systemic, such as by intravenous or intra-muscular administration, by administration to target cells ex-planted from a patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one siNA molecule of the instant invention. The expression vector can encode one or both strands of a siNA duplex, or a single self complementary strand that self hybridizes into a siNA duplex. The nucleic acid sequences encoding the siNA molecules of the instant invention can be operably linked in a manner that allows expression of the siNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siNA molecules of the instant invention; wherein said sequence is operably linked to said initiation region and said termination region, in a manner that allows expression and/or delivery of the siNA molecule. The vector can optionally include an open reading frame (ORF) for a protein operably linked on the 5′ side or the 3′-side of the sequence encoding the siNA of the invention; and/or an intron (intervening sequences).

Transcription of the siNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang, 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736). The above siNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising a nucleic acid sequence encoding at least one of the siNA molecules of the invention, in a manner that allows expression of that siNA molecule. The expression vector comprises in one embodiment; a) a transcription initiation region; b) a transcription termination region; and c) a nucleic acid sequence encoding at least one strand of the siNA molecule; wherein the sequence is operably linked to the initiation region and the termination region, in a manner that allows expression and/or delivery of the siNA molecule.

In another embodiment the expression vector comprises: a) a transcription initiation region; b) a transcription termination region; c) an open reading frame; and d) a nucleic acid sequence encoding at least one strand of a siNA molecule, wherein the sequence is operably linked to the 3′-end of the open reading frame; and wherein the sequence is operably linked to the initiation region, the open reading frame and the termination region, in a manner that allows expression and/or delivery of the siNA molecule. In yet another embodiment the expression vector comprises: a) a transcription initiation region; b) a transcription termination region; c) an intron; and d) a nucleic acid sequence encoding at least one siNA molecule; wherein the sequence is operably linked to the initiation region, the intron and the termination region, in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siNA molecule, wherein the sequence is operably linked to the 3′-end of the open reading frame; and wherein the sequence is operably linked to the initiation region, the intron, the open reading frame and the termination region, in a manner which allows expression and/or delivery of the siNA molecule.

### EXAMPLES

The following are non-limiting examples showing the selection, isolation, synthesis and activity of nucleic acids of the instant invention.

### Example 1

### Tandem Synthesis of siNA Constructs

Exemplary siNA molecules of the invention are synthesized in tandem using a cleavable linker, for example a succinyl-based linker. Tandem synthesis as described herein is followed by a one-step purification process that provides RNAi molecules in high yield. This approach is highly amenable to siNA synthesis in support of high throughput RNAi screening, and can be readily adapted to multi-column or multi-well synthesis platforms.

After completing a tandem synthesis of an siNA oligo and its compliment in which the 5′-terminal dimethoxytrityl (5′-O-DMT) group remains intact (trityl on synthesis), the oligonucleotides are deprotected as described above. Following deprotection, the siNA sequence strands are allowed to spontaneously hybridize. This hybridization yields a duplex in which one strand has retained the 5′-O-DMT group while the complementary strand comprises a terminal 5′-hydroxyl. The newly formed duplex behaves as a single molecule during routine solid-phase extraction purification (Trityl-On purification) even though only one molecule has a dimethoxytrityl group. Because the strands form a stable duplex, this dimethoxytrityl group (or an equivalent group, such as other trityl groups or other hydrophobic moieties) is all that is required to purify the pair of oligos, for example by using a C18 cartridge.

Standard phosphoramidite synthesis chemistry is used up to point of introducing a tandem linker, such as an inverted deoxy abasic succinate or glyceryl succinate linker (see FIG. 1) or an equivalent cleavable linker. A non-limiting example of linker coupling conditions that can be used includes a hindered base such as diisopropylethylamine (DIPA) and/or DMAP in the presence of an activator reagent such as Bromotripyrrolidinophosphoniumhexaflurorophosphate (PyBrOP). After the linker is coupled, standard synthesis chemistry is utilized to complete synthesis of the second sequence leaving the terminal the 5′-O-DMT intact. Following synthesis, the resulting oligonucleotide is deprotected according to the procedures described herein and quenched with a suitable buffer, for example with 50 mM NaOAc or 1.5M NH₄H₂CO₃.

Purification of the siNA duplex can be readily accomplished using solid phase extraction, for example using a Waters C18 SepPak 1 g cartridge conditioned with 1 column volume (CV) of acetonitrile, 2 CV H2O, and 2 CV 50 mM NaOAc. The sample is loaded and then washed with 1 CV H2O or 50 mM NaOAc. Failure sequences are eluted with 1 CV 14% ACN (Aqueous with 50 mM NaOAc and 50 mM NaCl). The column is then washed, for example with 1 CV H2O followed by on-column detritylation, for example by passing 1 CV of 1% aqueous trifluoroacetic acid (TFA) over the column, then adding a second CV of 1% aqueous TFA to the column and allowing to stand for approx. 10 minutes. The remaining TFA solution is removed and the column washed with H2O followed by 1 CV 1M NaCl and additional H2O. The siNA duplex product is then eluted, for example using 1 CV 20% aqueous CAN.

FIG. 2 provides an example of MALDI-TOV mass spectrometry analysis of a purified siNA construct in which each peak corresponds to the calculated mass of an individual siNA strand of the siNA duplex. The same purified siNA provides three peaks when analyzed by capillary gel electrophoresis (CGE), one peak presumably corresponding to the duplex siNA, and two peaks presumably corresponding to the separate siNA sequence strands. Ion exchange HPLC analysis of the same siNA contract only shows a single peak. Testing of the purified siNA construct using a luciferase reporter assay described below demonstrated the same RNAi activity compared to siNA constructs generated from separately synthesized oligonucleotide sequence strands.

### Example 2

### Identification of Potential siNA Target Sites in any RNA Sequence

The sequence of an RNA target of interest, such as a viral or human mRNA transcript, is screened for target sites, for example by using a computer folding algorithm. In a non-limiting example, the sequence of a gene or RNA gene transcript derived from a database, such as Genbank, is used to generate siNA targets having complimentarily to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siNA molecules for efficacy, for example by using in vitro RNA cleavage assays, cell culture, or animal models. In a non-limiting example, anywhere from 1 to 1000 target sites are chosen within the transcript based on the size of the siNA contruct to be used. High throughput screening assays can be developed for screening siNA molecules using methods known in the art, such as with multi-well or multi-plate assays to determine efficient reduction in target gene expression.

### Example 3

### Selection of siNA Molecule Target Sites in a RNA

The following non-limiting steps can be used to carry out the selection of siNAs targeting a given gene sequence or transcipt.

1. The target sequence is parsed in silico into a list of all fragments or subsequences of a particular length, for example 23 nucleotide fragments, contained within the target sequence. This step is typically carried out using a custom Perl script, but commercial sequence analysis programs such as Oligo, MacVector, or the GCG Wisconsin Package can be employed as well.

2. In some instances the siNAs correspond to more than one target sequence; such would be the case for example in targeting many different strains of a viral sequence, for targeting different transcipts of the same gene, targeting different transcipts of more than one gene, or for targeting both the human gene and an animal homolog. In this case, a subsequence list of a particular length is generated for each of the targets, and then the lists are compared to find matching sequences in each list. The subsequences are then ranked according to the number of target sequences that contain the given subsequence; the goal is to find subsequences that are present in most or all of the target sequences. Alternately, the ranking can indentify subsequences that are unique to a target sequence, such as a mutant target sequence. Such an approach would enable the use of siNA to target specifically the mutant sequence and not effect the expression of the normal sequence.

3. In some instances the siNA subsequences are absent in one or more sequences while present in the desired target sequence; such would be the case if the siNA targets a gene with a paralogous family member that is to remain untargeted. As in case 2 above, a subsequence list of a particular length is generated for each of the targets, and then the lists are compared to find sequences that are present in the target gene but are absent in the untargeted paralog.

4. The ranked siNA subsequences can be further analyzed and ranked according to GC content. A preference can be given to sites containing 30-70% GC, with a further preference to sites containing 40-60% GC.

5. The ranked siNA subsequences can be further analyzed and ranked according to self-folding and internal hairpins. Weaker internal folds are preferred; strong hairpin structures are to be avoided.

6. The ranked siNA subsequences can be further analyzed and ranked according to whether they have runs of GGG or CCC in the sequence. GGG (or even more Gs) in either strand can make oligonucleotide synthesis problematic, so it is avoided whenever better sequences are available. CCC is searched in the target strand because that will place GGG in the antisense strand.

7. The ranked siNA subsequences can be further analyzed and ranked according to whether they have the dinucleotide UU (uridine dinucleotide) on the 3′-end of the sequence, and/or AA on the 5′-end of the sequence (to yield 3′ UU on the antisense sequence). These sequences allow one to design siNA molecules with terminal TT thymidine dinucleotides.

8. Four or five target sites are chosen from the ranked list of subsequences as described above. For example, in subsequences having 23 nucleotides, the right 21 nucleotides of each chosen 23-mer subsequence are then designed and synthesized for the upper (sense) strand of the siNA duplex, while the reverse complement of the left 21 nucleotides of each chosen 23-mer subsequence are then designed and synthesized for the lower (antisense) strand of the siNA duplex. If terminal TT residues are desired for the sequence (as described in paragraph 7), then the two 3′ terminal nucleotides of both the sense and antisense strands are replaced by TT prior to synthesizing the oligos.

9. The siNA molecules are screened in an in vitro, cell culture or animal model system to identify the most active siNA molecule or the most preferred target site within the target RNA sequence.

In an alternate approach, a pool of siNA constructs specific to an HBV target sequence is used to screen for target sites in cells expressing HBV RNA. The general strategy used in this approach is shown in FIG. 9. Cells expressing HBV (e.g., HEPG2) are transfected with the pool of siNA constructs and cells that demonstrate a phenotype associated with HBV inhibition are sorted. The pool of siNA constructs can be expressed from transcription cassettes inserted into appropriate vectors (see for example FIG. 7 and FIG. 8). Cells in which HBV expression is decreased due to siNA treatment demonstrate a phenotypic change, for example decreased production of HBV RNA or protein(s) compared to untreated cells or cells treated with a control siNA. The siNA from cells demonstrating a positive phenotypic change (e.g., decreased HBV RNA or protein), are sequenced to determine the most suitable target site(s) within the target RNA sequence.

### Example 4

### HBV Targeted siNA Design

siNA target sites were chosen by analyzing sequences of the HBV RNA target and generating a consensus HBV sequence based on a minimun 65% homology for sequences referred to by Genbank Accession Numbers in Table I. This way, conserved sequences encoding HBV are targeted by siNA molecules of the invention. Alternately, target sequences are chosen using the methodology described in Example 3. siNA molecules were designed that could bind each target and are optionally individually analyzed by computer folding to assess whether the siNA molecule can interact with the target sequence. Varying the length of the siNA molecules can be chosen to optimize activity. Generally, a sufficient number of complementary nucleotide bases are chosen to bind to, or otherwise interact with, the target RNA, but the degree of complementarity can be modulated to accommodate siNA duplexes or varying length or base composition. By using such methodologies, siNA molecules can be designed to target sites within any known RNA sequence, for example those RNA sequences corresponding to the any gene transcript.

### Example 5

### Chemical Synthesis and Purification of siNA

siNA molecules can be designed to interact with various sites in the RNA message, for example target sequences within the RNA sequences described herein. The sequence of one strand of the siNA molecule(s) is complementary to the target site sequences described above. The siNA molecules can be chemically synthesized using methods described herein. Inactive siNA molecules that are used as control sequences can be synthesized by scrambling the sequence of the siNA molecules such that it is not complementary to the target sequence.

### Example 6

### Models Used to Evaluate the Down-Regulation of HBV Gene Expression

Nucleic acid molecules targeted to the human HBV RNA are designed and synthesized as described above. These nucleic acid molecules can be tested for cleavage activity in vivo, for example, using the procedures described below. A variety of endpoints have been used in cell culture models to evaluate HBV-mediated effects after treatment with anti-HBV agents. Phenotypic endpoints include inhibition of cell proliferation, apoptosis assays and reduction of HBV RNA/protein expression. There are several methods by which these endpoints can be measured. For example, a nucleic acid-mediated decrease in the level of HBV RNA and/or HBV protein expression can be evaluated using methods known in the art, such as RT-PCR, Northern blot, ELISA, Western blot, and immunoprecipitation analyses, to name a few techniques.

Phenotypic Assays

Intracellular HBV gene expression can be assayed either by a Taqman® assay for HBV RNA or by ELISA for HBV protein. Extracellular virus can be assayed either by PCR for DNA or ELISA for protein. Antibodies are commercially available for HBV surface antigen and core protein. A secreted alkaline phosphatase expression plasmid can be used to normalize for differences in transfection efficiency and sample recovery. The method consists of coating a micro-titer plate with an antibody such as anti-HBsAg Mab (for example, Biostride B88-95-31ad,ay) at 0.1 to 10 μg/ml in a buffer (for example, carbonate buffer, such as Na₂CO₃ 15 mM, NaHCO₃ 35 mM, pH 9.5) at 4° C. overnight. The microtiter wells are then washed with PBST or the equivalent thereof, (for example, PBS, 0.05% Tween 20) and blocked for 0.1-24 hr at 37° C. with PBST, 1% BSA or the equivalent thereof. Following washing as above, the wells are dried (for example, at 37° C. for 30 min). Biotinylated goat anti-HBsAg or an equivalent antibody (for example, Accurate YVS1807) is diluted (for example at 1:1000) in PBST and incubated in the wells (for example, 1 hr. at 37° C.). The wells are washed with PBST (for example, 4×). A conjugate, (for example, Streptavidin/Alkaline Phosphatase Conjugate, Pierce 21324) is diluted to 10-10,000 ng/ml in PBST, and incubated in the wells (for example, 1 hr. at 37° C.). After washing as above, a substrate (for example, p-nitrophenyl phosphate substrate, Pierce 37620) is added to the wells, which are then incubated (for example, 1 hr. at 37° C.). The optical density is then determined (for example, at 405 nm). SEAP levels are then assayed, for example, using the Great EscAPe® Detection Kit (Clontech K2041-1), as per the manufacturer's instructions. In the above example, incubation times and reagent concentrations can be varied to achieve optimum results.

Cell Culture Models

HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA would be co-transfected with siNA molecules in cell culture. Such an approach has been used to report intracellular enzymatic nucleic acid molecule activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV.

Animal Models

The development of new antiviral agents for the treatment of chronic Hepatitis B has been aided by the use of animal models that are permissive to replication of related Hepadnaviridae such as Woodchuck Hepatitis Virus (WHV) and Duck Hepatitis Virus (DHV). In addition the use of transgenic mice has also been employed. Macejak et al., U.S. Ser. No. 60/335,059 (incorporated by reference herein in its entirety), describe a model in which the human hepatoblastoma cell line, HepG2.2.15, implanted as a subcutaneous (SC) tumor, was evaluated in terms of its usefulness in producing Hepatitis B viremia in mice. This model is useful for evaluating new HBV therapies such as siNA molecules described herein. The study showed that in mice bearing HepG2.2.15 SC tumors, HBV viremia was present. HBV DNA was detected in serum beginning on Day 35. Maximum serum viral levels reached 1.9×10⁵ copies/mL by day 49. This study also determined that the minimum tumor volume associated with viremia was 300 mm³. Therefore, the HepG2.2.15 cell line grown as a SC tumor produces a useful model of HBV viremia in mice. This model is suitable for evaluating siNA molecules of the invention targeting HBV RNA.

There are several other small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562). Transgenic mice that express HBV have also been used as a model to evaluate potential anti-virals. HBV DNA is detectable in both liver and serum (Morrey et al., 1999, Antiviral Res., 42, 97-108). An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22). Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

### Example 7

### Inhibition of HBV Using siNA Molecules of the Invention

Transfection of HepG2 Cells with psHB V-1 and siNA

The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-1 vector (Those skilled in the art understand that other methods can be used to generate a replication competent cDNA). This was done with an EcoRi and Hind III restriction digest. Following completion of the digest, a ligation was performed under dilute conditions (20 μg/ml) to favor intermolecular ligation. The total ligation mixture was then concentrated using Qiagen spin columns.

Transfection of the human hepatocellular carcinoma cell line, Hep G2, with replication-competent HBV DNA results in the expression of HBV proteins and the production of virions. To test the efficacy of siNAs targeted against HBV RNA, the siNA duplex (sense strand SEQ ID NO: 1338/antisense strand SEQ ID NO: 1342) was co-transfected with HBV genomic DNA twice at 25 nM, the first time with siNA and lipid 12.5 ug/ml and the second time with siNA and lipid at 2.5 ug/ml, 5.0 ug/ml, 7.5 ug/ml and 10 ug/ml, into Hep G2 cells, and the subsequent levels of secreted HBV surface antigen (HBsAg) were analyzed by ELISA. Inverted sequence duplexes were used as negative controls (sense strand SEQ ID NO: 1358/antisense strand SEQ ID NO: 1350). Alternately, the siNA duplex (sense strand SEQ ID NO: 1338/antisense strand SEQ ID NO: 1342) and (two right side colums in FIG. 11) was co-transfected with HBV genomic DNA once at 25 nM with lipid at 12.5 ug/ml into Hep G2 cells, and the subsequent levels of secreted HBV surface antigen (HBsAg) were analyzed by ELISA.

Analysis of HBsAg Levels Following siNA Treatment

Immulon 4 (Dynax) microtiter wells were coated overnight at 4° C. with anti-HBsAg Mab (Biostride B88-95-31ad,ay) at 1 μg/ml in Carbonate Buffer (Na2CO3 15 mM, NaHCO3 35 mM, pH 9.5). The wells were then washed 4× with PBST (PBS, 0.05% Tween® 20) and blocked for 1 hr at 37° C. with PBST, 1% BSA. Following washing as above, the wells were dried at 37° C. for 30 min. Biotinylated goat ant-HBsAg (Accurate YVS1807) was diluted 1:1000 in PBST and incubated in the wells for 1 hr. at 37° C. The wells were washed 4× with PBST. Streptavidin/Alkaline Phosphatase Conjugate (Pierce 21324) was diluted to 250 ng/ml in PBST, and incubated in the wells for 1 hr. at 37° C. After washing as above, p-nitrophenyl phosphate substrate (Pierce 37620) was added to the wells, which were then incubated for 1 hr. at 37° C. The optical density at 405 nm was then determined. Results of this study are summarized in FIG. 11, where the siNA duplex (sense strand SEQ ID NO: 1338/antisense strand SEQ ID NO: 1342) and inverted control siNA duplex (sense strand SEQ ID NO: 1358/antisense strand SEQ ID NO: 1350) were tested at differing lipid concentrations as indicated in the figure. As shown in FIG. 11, the siRNA construct targeting site 413 of HBV RNA provides significant inhibition of viral replication/activity when compared to an inverted siRNA control. This effect is seen consistently at differing concentrations of lipid transfection agent.

### Example 8

### RNAi In Vitro Assay to Assess siNA Activity

An in vitro assay that recapitulates RNAi in a cell free system is used to evaluate siNA constructs targeting HBV RNA targets. The assay comprises the system described by Tuschl et al., 1999, Genes and Development, 13, 3191-3197 and Zamore et al., 2000, Cell, 101, 25-33 adapted for use with HBV target RNA. A Drosophila extract derived from syncytial blastoderm is used to reconstitute RNAi activity in vitro. Target RNA is generated via in vitro transcription from an appropriate HBV expressing plasmid using T7 RNA polymerase or via chemical synthesis as described herein. Sense and antisense siNA strands (for example 20 uM each) are annealed by incubation in buffer (such as 100 mM potassium acetate, 30 mM HEPES-KOH, pH 7.4, 2 mM magnesium acetate) for 1 min. at 90° C. followed by 1 hour at 37° C., then diluted in lysis buffer (for example 100 mM potassium acetate, 30 mM HEPES-KOH at pH 7.4, 2 mM magnesium acetate). Annealing can be monitored by gel electrophoresis on an agarose gel in TBE buffer and stained with ethidium bromide. The Drosophila lysate is prepared using zero- to two-hour-old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 pM final concentration), and 10% [vol/vol] lysis buffer containing siNA (10 nM final concentration). The reaction mixture also contains 10 mM creatine phosphate, 10 ug.ml creatine phosphokinase, 100 um GTP, 100 uM UTP, 100 uM CTP, 500 uM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding RNA, then incubated at 25° C. for an additional 60 minutes. Reactions are quenched with 4 volumes of 1.25× Passive Lysis Buffer (Promega). Target RNA cleavage is assayed by RT-PCR analysis or other methods known in the art and are compared to control reactions in which siNA is omitted from the reaction.

Alternately, internally-labeled target RNA for the assay is prepared by in vitro transcription in the presence of [a-³²P] CTP, passed over a G 50 Sephadex column by spin chromatography and used as target RNA without further purification. Optionally, target RNA is 5′-³²P-end labeled using T4 polynucleotide kinase enzyme. Assays are performed as described above and target RNA and the specific RNA cleavage products generated by RNAi are visualized on an autoradiograph of a gel. The percentage of cleavage is determined by Phosphor Imager® quantitation of bands representing intact control RNA or RNA from control reactions without siNA and the cleavage products generated by the assay.

In one embodiment, this assay is used to determine target sites the HBV RNA target for siNA mediated RNAi cleavage, wherein a plurality of siNA constructs are screened for RNAi mediated cleavage of the HBV RNA target, for example by analyzing the assay reaction by electrophoresis of labeled target RNA, or by northern blotting, as well as by other methodologies well known in the art.

### Example 9

### Diagnostic Uses

The siNA molecules of the invention can be used in a variety of diagnostic applications, such as in identifying molecular targets such as RNA in a variety of applications, for example, in clinical, industrial, environmental, agricultural and/or research settings. Such diagnostic use of siNA molecules involves utilizing reconstituted RNAi systems, for example using cellular lysates or partially purified cellular lysates. siNA molecules of this invention can be used as diagnostic tools to examine genetic drift and mutations within diseased cells or to detect the presence of endogenous or exogenous, for example viral, RNA in a cell. The close relationship between siNA activity and the structure of the target RNA allows the detection of mutations in any region of the molecule, which alters the base-pairing and three-dimensional structure of the target RNA. By using multiple siNA molecules described in this invention, one can map nucleotide changes, which are important to RNA structure and function in vitro, as well as in cells and tissues. Cleavage of target RNAs with siNA molecules can be used to inhibit gene expression and define the role (essentially) of specified gene products in the progression of disease or infection. In this manner, other genetic targets can be defined as important mediators of the disease. These experiments will lead to better treatment of the disease progression by affording the possibility of combination therapies (e.g., multiple siNA molecules targeted to different genes, siNA molecules coupled with known small molecule inhibitors, or intermittent treatment with combinations siNA molecules and/or other chemical or biological molecules). Other in vitro uses of siNA molecules of this invention are well known in the art, and include detection of the presence of mRNAs associated with a disease, infection, or related condition. Such RNA is detected by determining the presence of a cleavage product after treatment with a siNA using standard methodologies, for example fluorescence resonance emission transfer (FRET).

In a specific example, siNA molecules that can cleave only wild-type or mutant forms of the target RNA are used for the assay. The first siNA molecules (i.e., those that can cleave only wild-type foms of target RNA) are used to identify wild-type RNA present in the sample and the second siNA molecules (i.e., those that can cleave only mutant forms of target RNA) will be used to identify mutant RNA in the sample. As reaction controls, synthetic substrates of both wild-type and mutant RNA will be cleaved by both siNA molecules to demonstrate the relative siNA efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two siNA molecules, two substrates, and one unknown sample, which will be combined into six reactions. The presence of cleavage products will be determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., disease related or infection related) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
HBV Sequences

| | | Accession | |
|---|---|---|---|
| Seq Name | Total Score | No. | LOCUS |
| gi\|5019947\|gb\|AF143301.1\|AF143301 | 230559 | AF143301.1 | AF143301 |
| gi\|6063432\|dbj\|AB033552.1\|AB033552 | 292977 | AB033552.1 | AB033552 |
| gi\|12060433\|dbj\|AB049609.1\| | 267942 | AB049609.1 | |
| gi\|6692481\|gb\|AF121242.1\|AF121242 | 228783 | AF121242.1 | AF121242 |
| gi\|21280241\|dbj\|AB073828.1\| | 307127 | AB073828.1 | |
| gi\|1107590\|emb\|X80925.1\|HBVP6PCXX | 208895 | X80925.1 | HBVP6PCXX |
| gi\|13491148\|gb\|AF330110.1\|AF330110 | 295703 | AF330110.1 | AF330110 |
| gi\|21280285\|dbj\|AB073850.1\| | 250338 | AB073850.1 | |
| gi\|21326584\|ref\|NC_003977.1\| | 292733 | NC_003977.1 | |
| gi\|19568072\|gb\|AY077736.1\| | 159292 | AY077736.1 | |
| gi\|1914688\|emb\|X98073.1\|HBVCGINCX | 272903 | X98073.1 | HBVCGINCX |
| gi\|9454475\|gb\|AF282918.1\|AF282918 | 296123 | AF282918.1 | AF282918 |
| gi\|21280269\|dbj\|AB073842.1\| | 261966 | AB073842.1 | |
| gi\|329628\|gb\|M54923.1\|HPBADWZ | 251770 | M54923.1 | HPBADWZ |
| gi\|6009770\|dbj\|AB026813.1\|AB026813 | 302607 | AB026813.1 | AB026813 |
| gi\|19568077\|gb\|AY077735.1\| | 169063 | AY077735.1 | |
| gi\|21280295\|dbj\|AB073855.1\| | 245594 | AB073855.1 | |
| gi\|452637\|emb\|X75658.1\|HHVBFFOU | 170005 | X75658.1 | HHVBFFOU |
| gi\|11191867\|dbj\|AB036909.1\|AB036909 | 184492 | AB036909.1 | AB036909 |
| gi\|15425688\|dbj\|AB056513.1\| | 185259 | AB056513.1 | |
| gi\|6692485\|gb\|AF121246.1\|AF121246 | 300222 | AF121246.1 | AF121246 |
| gi\|21280279\|dbj\|AB073847.1\| | 251950 | AB073847.1 | |
| gi\|5019984\|gb\|AF143308.1\|AF143308 | 225170 | AF143308.1 | AF143308 |
| gi\|10441111\|gb\|AF182804.1\|AF182804 | 262414 | AF182804.1 | AF182804 |
| gi\|5019979\|gb\|AF143307.1 \|AF143307 | 227324 | AF143307.1 | AF143307 |
| gi\|10441106\|gb\|AF182803.1\|AF182803 | 264971 | AF182803.1 | AF182803 |
| gi\|10443814\|gb\|AF241408.1\|AF241408 | 248822 | AF241408.1 | AF241408 |
| gi\|15419852\|gb\|AF297624.1\|AF297624 | 248555 | AF297624.1 | AF297624 |
| gi\|6063467\|dbj\|AB033559.1\|AB033559 | 220175 | AB033559.1 | AB033559 |
| gi\|15419847\|gb\|AF297623.1\|AF297623 | 218711 | AF297623.1 | AF297623 |
| gi\|11071965\|dbj\|A8042283.1\|AB042283 | 289018 | AB042283.1 | AB042283 |
| gi\|5114069\|gb\|AF090839.1\|AF090839 | 250372 | AF090839.1 | AF090839 |
| gi\|5019943\|gb\|AF143300.1\|AF143300 | 223920 | AF143300.1 | AF143300 |
| gi\|6063422\|dbj\|AB033550.1\|AB033550 | 298540 | AB033550.1 | AB033550 |
| gi\|1914693\|emb\|X98072.1\|HBVCGINSC | 274513 | X98072.1 | HBVCGINSC |
| gi\|12060438\|dbj\|AB049610.1\| | 255954 | AB049610.1 | |
| gi\|21280227\|dbj\|AB073821.1\| | 286641 | AB073821.1 | |
| gi\|11191835\|dbj\|AB036905.1\|AB036905 | 184492 | AB036905.1 | AB036905 |
| gi\|11191851\|dbj\|AB036907.1\|AB036907 | 183325 | AB036907.1 | AB036907 |
| gi\|15778318\|gb\|AF411408.1\|AF411408 | 286382 | AF411408.1 | AF411408 |
| gi\|21280253\|dbj\|AB073834.1\| | 309461 | AB073834.1 | |
| gi\|560062\|dbj\|D23678.1\|HPBA2HYS2 | 261916 | D23678.1 | HPBA2HYS2 |
| gi\|10443830\|gb\|AF241410.1\|AF241410 | 244968 | AF241410.1 | AF241410 |
| gi\|6009760\|dbj\|AB026811.1\|AB026811 | 303731 | AB026811.1 | AB026811 |
| gi\|21280237\|dbj\|AB073826.1\| | 306003 | AB073826.1 | |
| gi\|1914710\|emb\|X98075.1\|HBVDEFVP2 | 282323 | X98075.1 | HBVDEFVP2 |
| gi\|1914704\|emb\|X98074.1\|HBVDEFVP1 | 259015 | X98074.1 | HBVDEFVP1 |
| gi\|1914699\|emb\|X98077.1\|HBVCGWITY | 285934 | X98077.1 | HBVCGWITY |
| gi\|18621111\|emb\|AJ344116.1\|HEB344116 | 218625 | AJ344116.1 | HEB344116 |
| gi\|21280263\|dbj\|AB073839.1\| | 274633 | AB073839.1 | |
| gi\|2182117\|gb\|U95551.1\|U95551 | 222999 | U95551.1 | U95551 |
| gi\|18845081\|gb\|AF473543.1\| | 275269 | AF473543.1 | |
| gi\|11191939\|dbj\|AB036918.1\|AB036918 | 180426 | AB036918.1 | AB036918 |
| gi\|18146697\|dbj\|AB064316.1\| | 164182 | AB064316.1 | |
| gi\|221497\|dbj\|D00329.1\|HPBADW1 | 269342 | D00329.1 | HPBADW1 |
| gi\|6063457\|dbj\|AB033557.1\|AB033557 | 276860 | AB033557.1 | AB033557 |
| gi\|807711\|dbj\|D50489.1\|HPBA11A | 271115 | D50489.1 | HPBA11A |
| gi\|6692478\|gb\|AF121239.1\|AF121239 | 222516 | AF121239.1 | AF121239 |
| gi\|12246985\|gb\|AF223958.1\|AF223958 | 277556 | AF223958.1 | AF223958 |
| gi\|4140292\|emb\|AJ131956.1\|HBV131956 | 223626 | AJ131956.1 | HBV131956 |
| gi\|13991863\|gb\|AF363961.1\|AF363961 | 287052 | AF363961.1 | AF363961 |
| gi\|5019931\|gb\|AF143298.1\|AF143298 | 234175 | AF143298.1 | AF143298 |
| gi\|12585611\|gb\|M57663.2\|HPBADWZCG | 213646 | M57663.2 | HPBADWZCG |
| gi\|1814218\|gb\|U46935.1\|HBU46935 | 170791 | U46935.1 | HBU46935 |
| gi\|4323201\|gb\|AF100309.1\| | 291448 | AF100309.1 | |
| gi\|15778322\|gb\|AF411409.1\|AF411409 | 274184 | AF411409.1 | AF411409 |
| gi\|11191907\|dbj\|AB036914.1\|AB036914 | 184305 | AB036914.1 | AB036914 |
| gi\|9454470\|gb\|AF282917.1\|AF282917 | 278685 | AF282917.1 | AF282917 |
| gi\|12247041\|gb\|AF223965.1\|AF223965 | 174200 | AF223965.1 | AF223965 |
| gi\|13365550\|dbj\|AB048705.1\|AB048705 | 221176 | AB048705.1 | AB048705 |
| gi\|11191923\|dbj\|AB036916.1\|AB036916 | 184242 | AB036916.1 | AB036916 |
| gi\|21280301\|dbj\|AB073858.1\| | 246498 | AB073858.1 | |
| gi\|21280265\|dbj\|AB073840.1\| | 293680 | AB073840.1 | |
| gi\|16751309\|gb\|AY057948.1\| | 265183 | AY057948.1 | |
| gi\|6692479\|gb\|AF121240.1\|AF121240 | 237011 | AF121240.1 | AF121240 |
| gi\|6692482\|gb\|AF121243.1\|AF121243 | 300260 | AF121243.1 | AF121243 |
| gi\|21280291\|dbj\|\|AB073853.1\| | 217945 | AB073853.1 | |
| gi\|21280249\|dbj\|AB073832.1\| | 255818 | AB073832.1 | |
| gi\|21624227\|dbj\|AB074755.1\| | 271043 | AB074755.1 | |
| gi\|21280275\|dbj\|AB073845.1\| | 257829 | AB073845.1 | |
| gi\|20800457\|gb\|U87746.3\| | 220314 | U87746.3 | |
| gi\|5019974\|gb\|AF143306.1\|AF143306 | 220769 | AF143306.1 | AF143306 |
| gi\|18146685\|dbj\|AB064314.1\| | 247361 | AB064314.1 | |
| gi\|21280259\|dbj\|AB073837.1\| | 290908 | AB073837.1 | |
| gi\|10441101\|gb\|AF182802.1\|AF182802 | 255610 | AF182802.1 | AF182802 |
| gi\|1155012\|emb\|X51970.1\|HVHEPB | 230814 | X51970.1 | HVHEPB |
| gi\|6063447\|dbj\|AB033555.1\|AB033555 | 258566 | AB033555.1 | AB033555 |
| gi\|4490403\|emb\|Y18857.1\|HBV18857 | 274668 | Y18857.1 | HBV18857 |
| gi\|4468847\|emb\|AJ131133.1\|HBV131133 | 273880 | AJ131133.1 | HBV131133 |
| gi\|15419842\|gb\|AF297622.1\|AF297622 | 232545 | AF297622.1 | AF297622 |
| gi\|21431678\|gb\|U87747.3\| | 273379 | U87747.3 | |
| gi\|6692486\|gb\|AF121247.1\|AF121247 | 284619 | AF121247.1 | AF121247 |
| gi\|15419837\|gb\|AF297621.1\|AF297621 | 221472 | AF297621.1 | AF297621 |
| gi\|329667\|gb\|M32138.1\|HPBHBVAA | 215731 | M32138.1 | HPBHBVAA |
| gi\|5114064\|gb\|AF090838.1\|AF090838 | 222057 | AF090838.1 | AF090838 |
| gi\|12247001\|gb\|AF223960.1\|AF223960 | 271086 | AF223960.1 | AF223960 |
| gi\|18389985\|gb\|AF462041.1\| | 250341 | AF462041.1 | |
| gi\|560087\|dbj\|D23683.1\|HPBC5HKO2 | 247233 | D23683.1 | HPBC5HKO2 |
| gi\|14334406\|gb\|AY034878.1\| | 214004 | AY034878.1 | |
| gi\|11935071\|gb\|AF305327.1\|AF305327 | 181882 | AF305327.1 | AF305327 |
| gi\|59455\|emb\|X70185.1\|HBVXCPS | 255514 | X70185.1 | HBVXCPS |
| gi\|11191875\|dbj\|AB036910.1\|AB036910 | 179530 | AB036910.1 | AB036910 |
| gi\|18252538\|gb\|AF458665.1\|AF458665 | 256655 | AF458665.1 | AF458665 |
| gi\|12247017\|gb\|AF223962.1\|AF223962 | 171583 | AF223962.1 | AF223962 |
| gi\|11191891\|dbj\|AB036912.1\|AB036912 | 184598 | AB036912.1 | AB036912 |
| gi\|21280233\|dbj\|AB073824.1\| | 273493 | AB073824.1 | |
| gi\|15425700\|dbj\|AB056516.1\| | 172653 | AB056516.1 | |
| gi\|12060183\|dbj\|AB037927.1\|AB037927 | 161772 | AB037927.1 | AB037927 |
| gi\|6692490\|gb\|AF121251.1\|AF121251 | 281174 | AF121251.1 | AF121251 |
| gi\|13365546\|dbj\|AB048703.1\|AB048703 | 221017 | AB048703.1 | AB048703 |
| gi\|18252589\|gb\|AF461043.1\|AF461043 | 276940 | AF461043.1 | AF461043 |
| gi\|451966\|gb\|L27106.1\|HPBMUT | 199839 | L27106.1 | HPBMUT |
| gi\|21280243\|dbj\|AB073829.1\| | 304269 | AB073829.1 | |
| gi\|18146673\|dbj\|AB064312.1\| | 181001 | AB064312.1 | |
| gi\|15211885\|emb\|AJ309369.1\|HEB309369 | 233378 | AJ309369.1 | HEB309369 |
| gi\|6063437\|dbj\|AB033553.1\|AB033553 | 293149 | AB033553.1 | AB033553 |
| gi\|21280287\|dbj\|AB073851.1\| | 256764 | AB073851.1 | |
| gi\|19849032\|gb\|AF405706.1\| | 186584 | AF405706.1 | |
| gi\|5019968\|gb\|AF143305.1\|AF143305 | 219149 | AF143305.1 | AF143305 |
| gi\|21280297\|dbj\|AB073856.1\| | 253274 | AB073856.1 | |
| gi\|474959\|gb\|M12906.1\|HPBADRA | 283060 | M12906.1 | HPBADRA |
| gi\|6009775\|dbj\|AB026814.1\|AB026814 | 303971 | AB026814.1 | AB026814 |
| gi\|4490393\|emb\|Y18855.1\|HBV18855 | 274699 | Y18855.1 | HBV18855 |
| gi\|10443806\|gb\|AF241407.1\|AF241407 | 248499 | AF241407.1 | AF241407 |
| gi\|4490408\|emb\|Y18858.1\|HBV18858 | 275693 | Y18858.1 | HBV18858 |
| gi\|527440\|emb\|Z35717.1\|HBVGEN2 | 253405 | Z35717.1 | HBVGEN2 |
| gi\|5211892\|emb\|AJ309370.1\|HEB309370 | 235466 | AJ309370.1 | HEB309370 |
| gi\|5257487\|gb\|AF151735.1\|AF151735 | 227289 | AF151735.1 | AF151735 |
| gi\|329649\|gb\|M38636.1\|HPBCGADR | 261795 | M38636.1 | HPBCGADR |
| gi\|11071967\|dbj\|AB042284.1\|AB042284 | 281386 | AB042284.1 | AB042284 |
| gi\|16751304\|gb\|AY057947.1\| | 292195 | AY057947.1 | |
| gi\|560067\|dbj\|D23679.1\|HPBA3HMS2 | 270945 | D23679.1 | HPBA3HMS2 |
| gi\|21280245\|dbj\|AB073830.1\| | 271145 | AB073830.1 | |
| gi\|18146661\|dbj\|AB064310.1\| | 179324 | AB064310.1 | |
| gi\|560077\|dbj\|D23681.1\|HPBC4HST2 | 258955 | D23681.1 | HPBC4HST2 |
| gi\|21280271\|dbj\|AB073843.1\| | 264504 | AB073843.1 | |
| gi\|21280229\|dbj\|AB073822.1\| | 295616 | AB073822.1 | |
| gi\|1359675\|emb\|X97848.1\|HBVP2CSX | 226618 | X97848.1 | HBVP2CSX |
| gi\|6063427\|dbj\|AB033551.1\|AB033551 | 291777 | AB033551.1 | AB033551 |
| gi\|21280255\|dbj\|AB073835.1\| | 264943 | AB073835.1 | |
| gi\|6692483\|gb\|AF121244.1\|AF121244 | 301914 | AF121244.1 | AF121244 |
| gi\|21280281\|dbj\|AB073848.1\| | 279357 | AB073848.1 | |
| gi\|21280239\|dbj\|AB073827.1\| | 307559 | AB073827.1 | |
| gi\|15425692\|dbj\|AB056514.1\| | 184194 | AB056514.1 | |
| gi\|12246961\|gb\|AF223955.1\|AF223955 | 282408 | AF223955.1 | AF223955 |
| gi\|10934053\|dbj\|AB050018.1\|AB050018 | 298098 | AB050018.1 | AB050018 |
| gi\|18032031\|gb\|AY066028.1\| | 285293 | AY066028.1 | |
| gi\|19224211\|gb\|AF479684.1\| | 293124 | AF479684.1 | |
| gi\|6009765\|dbj\|AB026812.1\|AB026812 | 302802 | AB026812.1 | AB026812 |
| gi\|11191859\|dbj\|AB036908.1\|AB036908 | 179271 | AB036908.1 | AB036908 |
| gi\|1514493\|emb\|Y07587.1\|HBVAYWGEN | 234664 | Y07587.1 | HBVAYWGEN |
| gi\|10443838\|gb\|AF241411.1\|AF241411 | 247518 | AF241411.1 | AF241411 |
| gi\|18621118\|emb\|AJ344117.1\|HEB344117 | 222960 | AJ344117.1 | HEB344117 |
| gi\|6692487\|gb\|AF121248.1\|AF121248 | 286004 | AF121248.1 | AF121248 |
| gi\|12246977\|gb\|AF223957.1\|AF223957 | 285866 | AF223957.1 | AF223957 |
| gi\|18252533\|gb\|AF458664.1\|AF458664 | 299135 | AF458664.1 | AF458664 |
| gi\|527435\|emb\|Z35716.1\|HBVGEN1 | 231786 | Z35716.1 | HBVGEN1 |
| gi\|4490398\|emb\|Y18856.1\|HBV18856 | 269371 | Y18856.1 | HBV18856 |
| gi\|4323196\|gb\|AF100308.1\|AF100308 | 295195 | AF100308.1 | AF100308 |
| gi\|13365542\|dbj\|AB048701.1\|AB048701 | 203430 | AB048701.1 | AB048701 |
| gi\|4206634\|gb\|AF068756.1\|AF068756 | 278450 | AF068756.1 | AF068756 |
| gi\|12247033\|gb\|AF223964.1\|AF223964 | 165757 | AF223964.1 | AF223964 |
| gi\|11191843\|dbj\|AB036906.1\|AB036906 | 183325 | AB036906.1 | AB036906 |
| gi\|11877208\|emb\|AJ132335.1\|HBV132335 | 201764 | AJ132335.1 | HBV132335 |
| gi\|1107583\|emb\|X80926.1\|HBVP5PCXX | 219450 | X80926.1 | HBVP5PCXX |
| gi\|5019963\|gb\|AF143304.1\|AF143304 | 241689 | AF143304.1 | AF143304 |
| gi\|21280267\|dbj\|AB073841.1\| | 277079 | AB073841.1 | |
| gi\|2288869\|dbj\|D28880.1\|D28880 | 255723 | D28880.1 | D28880 |
| gi\|5019958\|gb\|AF143303.1\|AF143303 | 220264 | AF143303.1 | AF143303 |
| gi\|18621103\|emb\|AJ344115.1\|HEB344115 | 201900 | AJ344115.1 | HEB344115 |
| gi\|21280293\|dbj\|AB073854.1\| | 241071 | AB073854.1 | |
| gi\|18031713\|gb\|AY033073.1\| | 267088 | AY033073.1 | |
| gi\|14485224\|gb\|AF384372.1\|AF384372 | 276619 | AF384372.1 | AF384372 |
| gi\|5114084\|gb\|AF090842.1\|AF090842 | 214699 | AF090842.1 | AF090842 |
| gi\|21280277\|dbj\|AB073846.1\| | 266691 | AB073846.1 | |
| gi\|5114079\|gb\|AF090841.1\|AF090841 | 232925 | AF090841.1 | AF090841 |
| gi\|18031708\|gb\|AY033072.1\| | 270012 | AY033072.1 | |
| gi\|11191947\|dbj\|AB036919.1\|AB036919 | 184078 | AB036919.1 | AB036919 |
| gi\|221505\|dbj\|D00220.1\|HPBVCG | 178041 | D00220.1 | HPBVCG |
| gi\|6063462\|dbj\|AB033558.1\|AB033558 | 197912 | AB033558.1 | AB033558 |
| gi\|541655\|dbj\|D16665.1\|HPBADRM | 253402 | D16665.1 | HPBADRM |
| gi\|11071963\|dbj\|AB042282.1\|AB042282 | 283035 | AB042282.1 | AB042282 |
| gi\|10443822\|gb\|AF241409.1\|AF241409 | 226800 | AF241409.1 | AF241409 |
| gi\|15778336\|gb\|AF411412.1\|AF411412 | 263874 | AF411412.1 | AF411412 |
| gi\|1359690\|emb\|X97850.1\|HBVP4CSX | 253482 | X97850.1 | HBVP4CSX |
| gi\|313780\|emb\|X59795.1\|HBVAYWMCG | 214427 | X59795.1 | HBVAYWMCG |
| gi\|12247009\|gb\|AF223961.1\|AF223961 | 274267 | AF223961.1 | AF223961 |
| gi\|6692480\|gb\|AF121241.1\|AF121241 | 236801 | AF121241.1 | AF121241 |
| gi\|21280251\|dbj\|AB073833.1\| | 294823 | AB073833.1 | |
| gi\|11191915\|dbj\|AB036915.1\|AB036915 | 183839 | AB036915.1 | AB036915 |
| gi\|21280235\|dbj\|AB073825.1\| | 266911 | AB073825.1 | |
| gi\|11191931\|dbj\|AB036917.1\|AB036917 | 184242 | AB036917.1 | AB036917 |
| gi\|1107576\|emb\|X80924.1\|HBVP4PCXX | 218080 | X80924.1 | HBVP4PCXX |
| gi\|221500\|dbj\|D12980.1\|HPBCG | 289726 | D12980.1 | HPBCG |
| gi\|21280261\|dbj\|AB073838.1\| | 248223 | AB073838.1 | |
| gi\|13365548\|dbj\|AB048704.1\|AB048704 | 218626 | AB048704.1 | AB048704 |
| gi\|21624234\|dbj\|AB074756.1\| | 270134 | AB074756.1 | |
| gi\|11191899\|dbj\|AB036913.1\|AB036913 | 177808 | AB036913.1 | AB036913 |
| gi\|14290239\|gb\|AF384371.1\|AF384371 | 285891 | AF384371.1 | AF384371 |
| gi\|18146691\|dbj\|AB064315.1\| | 121870 | AB064315.1 | |
| gi\|15419830\|gb\|AF297620.1\|AF297620 | 237532 | AF297620.1 | AF297620 |
| gi\|6983934\|gb\|AF160501.1\|AF160501 | 182351 | AF160501.1 | AF160501 |
| gi\|21388705\|dbj\|AB074047.1\| | 244069 | AB074047.1 | |
| gi\|6063452\|dbj\|AB033556.1\|AB033556 | 290959 | AB033556.1 | AB033556 |
| gi\|6692484\|gb\|AF121245.1\|AF121245 | 300260 | AF121245.1 | AF121245 |
| gi\|21280289\|dbj\|AB073852.1\| | 246774 | AB073852.1 | |
| gi\|18146679\|dbj\|AB064313.1\| | 175470 | AB064313.1 | |
| gi\|221499\|dbj\|D00331.1\|HPBADW3 | 252986 | D00331.1 | HPBADW3 |
| gi\|15211900\|emb\|AJ309371.1\|HEB309371 | 239614 | AJ309371.1 | HEB309371 |
| gi\|560082\|dbj\|D23682.1\|HPBB5HKO1 | 255559 | D23682.1 | HPBB5HKO1 |
| gi\|21280299\|dbj\|AB073857.1\| | 247755 | AB073857.1 | |
| gi\|15778332\|gb\|AF411411.1\|AF411411 | 286382 | AF411411.1 | AF411411 |
| gi\|15778327\|gb\|AF411410.1\|AF411410 | 267992 | AF411410.1 | AF411410 |
| gi\|12246993\|gb\|AF223959.1\|AF223959 | 279509 | AF223959.1 | AF223959 |
| gi\|2829154\|gb\|AF043594.1\|AF043594 | 223502 | AF043594.1 | AF043594 |
| gi\|6692488\|gb\|AF121249.1\|AF121249 | 305912 | AF121249.1 | AF121249 |
| gi\|10441116\|gb\|AF182805.1 51 AF182805 | 265419 | AF182805.1 | AF182805 |
| gi\|15419857\|gb\|AF297625.1\|AF297625 | 216580 | AF297625.1 | AF297625 |
| gi\|11191883\|dbj\|AB036911.1\|AB036911 | 184393 | AB036911.1 | AB036911 |
| gi\|5019937\|gb\|AF143299.1\|AF143299 | 235942 | AF143299.1 | AF143299 |
| gi\|11071969\|dbj\|AB042285.1\|AB042285 | 257613 | AB042285.1 | AB042285 |
| gi\|12060190\|dbj\|AB037928.1\|AB037928 | 168481 | AB037928.1 | AB037928 |
| gi\|21280247\|dbj\|AB073831.1\| | 287868 | AB073831.1 | |
| gi\|1359682\|emb\|X97849.1\|HBVP3CSX | 218388 | X97849.1 | HBVP3CSX |
| gi\|329616\|gb\|M38454.1\|HPBADR1CG | 272026 | M38454.1 | HPBADR1CG |
| gi\|21280273\|dbj\|AB073844.1\| | 261056 | AB073844.1 | |
| gi\|1359698\|emb\|X97851.1\|HBVP6CSX | 295810 | X97851.1 | HBVP6CSX |
| gi\|6063442\|dbj\|AB033554.1\|AB033554 | 257344 | AB033554.1 | AB033554 |
| gi\|5114074\|gb\|AF090840.1\|AF090840 | 247925 | AF090840.1 | AF090840 |
| gi\|6692489\|gb\|AF121250.1\|AF121250 | 294273 | AF121250.1 | AF121250 |
| gi\|18146667\|dbj\|AB064311.1\| | 179163 | AB064311.1 | |
| gi\|21280257\|dbj\|AB073836.1\| | 264148 | AB073836.1 | |
| gi\|12246953\|gb\|AF223954.1\|AF223954 | 285309 | AF223954.1 | AF223954 |
| gi\|9082083\|gb\|AF233236.1\|AF233236 | 282508 | AF233236.1 | AF233236 |
| gi\|288927\|emb\|X72702.1\|HBVORFS | 225307 | X72702.1 | HBVORFS |
| gi\|221498\|dbj\|D00330.1\|HPBADW2 | 299926 | D00330.1 | HPBADW2 |
| gi\|21280283\|dbj\|AB073849.1\| | 230392 | AB073849.1 | |
| gi\|1914716\|emb\|X98076.1\|HBVDEFVP3 | 228252 | X98076.1 | HBVDEFVP3 |
| gi\|15425696\|dbj\|AB056515.1\| | 180333 | AB056515.1 | |
| gi\|560057\|dbj\|D23677.1\|HPBA1HKK2 | 269146 | D23677.1 | HPBA1HKK2 |
| gi\|6009780\|dbj\|AB026815.1\|AB026815 | 294151 | AB026815.1 | AB026815 |
| gi\|12246969\|gb\|AF223956.1\|AF223956 | 278063 | AF223956.1 | AF223956 |
| gi\|15072539\|gb\|AY040627.1\| | 287659 | AY040627.1 | |
| gi\|2829148\|gb\|AF043593.1\|AF043593 | 226770 | AF043593.1 | AF043593 |
| gi\|560072\|dbj\|D23680.1\|HPBB4HST1 | 279791 | D23680.1 | HPBB4HST1 |
| gi\|13991873\|gb\|AF363963.1\|AF363963 | 279252 | AF363963.1 | AF363963 |
| gi\|560092\|dbj\|D23684.1\|HPBC6T588 | 291474 | D23684.1 | HPBC6T588 |
| gi\|21280231\|dbj\|AB073823.1\| | 279375 | AB073823.1 | |
| gi\|11191955\|dbj\|AB036920.1\|AB036920 | 183088 | AB036920.1 | AB036920 |
| gi\|15419825\|gb\|AF297619.1\|AF297619 | 231156 | AF297619.1 | AF297619 |
| gi\|13991868\|gb\|AF363962.1\|AF363962 | 259635 | AF363962.1 | AF363962 |
| gi\|20151226\|gb\|U87742.3\| | 203103 | U87742.3 | |
| gi\|13365544\|dbj\|AB048702.1\|AB048702 | 219013 | AB048702.1 | AB048702 |
| gi\|5019952\|gb\|AF143302.1\|AF143302 | 229555 | AF143302.1 | AF143302 |
| gi\|12247025\|gb\|AF223963.1\|AF223963 | 161165 | AF223963.1 | AF223963 |

TABLE II

HBV siRNA and Target Sequences

| | | | | | |
|---|---|---|---|---|---|
| Sequence | Seq ID | Upper seq | Seq ID | Lower seq | Seq ID |

| | | | | | |
|---|---|---|---|---|---|
| GAUCCUGCUGCUAUGCCUC | 1 | CAUCCUGCUGCUAUGCCUC | 1 | GAGGCAUAGCAGCAGGAUG | 647 |
| AUCCUGCUGCUAUGCCUCA | 2 | AUCCUGCUGCUAUGCCUCA | 2 | UGAGGCAUAGCAGCAGGAU | 648 |
| GGCUGUAGGCAUAAAUUGG | 3 | GGCUGUAGGCAUAAAUUGG | 3 | CCAAUUUAUGCCUACAGCC | 649 |
| GCUGUAGGCAUAAAUUGGU | 4 | GCUGUAGGCAUAAAUUGGU | 4 | ACCAAUUUAUGCCUACAGC | 650 |
| GCUGCUAUGCCUCAUCUUC | 5 | GCUGCUAUGCCUCAUCUUC | 5 | GAAGAUGAGGCAUAGCAGC | 651 |
| UGCUAUGCCUCAUCUUCUU | 6 | UGCUAUGCCUCAUCUUCUU | 6 | AAGAAGAUGAGGCAUAGCA | 652 |
| UGCUGCUAUGCCUCAUCUU | 7 | UGCUGCUAUGCCUCAUCUU | 7 | AAGAUGAGGCAUAGCAGCA | 653 |
| CUGCUAUGCCUCAUCUUCU | 8 | CUGCUAUGCCUCAUCUUCU | 8 | AGAAGAUGAGGCAUAGCAG | 654 |
| CUGCUGCUAUGCCUCAUCU | 9 | CUGCUGCUAUGCCUGAUCU | 9 | AGAUGAGGCAUAGCAGCAG | 655 |
| UCCUGCUGCUAUGCCUCAU | 10 | UCCUGCUGCUAUGCCUCAU | 10 | AUGAGGCAUAGCAGCAGGA | 656 |
| CCUGCUGCUAUGCCUCAUC | 11 | CCUGCUGCUAUGCCUCAUC | 11 | GAUGAGGCAUAGCAGCAGG | 657 |
| GAAGAAGAACUCCCUCGCC | 12 | GAAGAAGAACUCCCUCGCC | 12 | GGCGAGGGAGUUCUUCUUC | 658 |
| GAGGCUGUAGGCAUAAAUU | 13 | GAGGCUGUAGGCAUAAAUU | 13 | AAUUUAUGCCUACAGCCUC | 659 |
| AGGCUGUAGGCAUAAAUUG | 14 | AGGCUGUAGGCAUAAAUUG | 14 | CAAUUUAUGCCUACAGCCU | 660 |
| GGAGGCUGUAGGCAUAAAU | 15 | GGAGGCUGUAGGCAUAAAU | 15 | AUUUAUGCCUACAGCCUCC | 661 |
| AAGCCUCCAAGCUGUGCCU | 16 | AAGCCUCCAAGCUGUGCCU | 16 | AGGCACAGCUUGGAGGCUU | 662 |
| CCUCCAAGCUGUGCCUUGG | 17 | CCUCCAAGCUGUGCCUUGG | 17 | CCAAGGCACAGCUUGGAGG | 663 |
| GAAGAACUCCCUCGCCUCG | 18 | GAAGAACUCCCUCGCCUCG | 18 | CGAGGCGAGGGAGUUCUUC | 664 |
| UCAAGCCUCCAAGCUGUGC | 19 | UCAAGCCUCCAAGCUGUGC | 19 | GCACAGCUUGGAGGCUUGA | 665 |
| UCGUGGUGGACUUCUCUCA | 20 | UCGUGGUGGACUUCUCUCA | 20 | UGAGAGAAGUCCACCACGA | 666 |
| CUCGUGGUGGACUUCUCUC | 21 | CUCGUGGUGGACUUCUCUC | 21 | GAGAGAAGUCCACCACGAG | 667 |
| AAGAACUCCCUCGCCUCGC | 22 | AAGAACUCCCUCGCCUCGC | 22 | GCGAGGCGAGGGAGUUCUU | 668 |
| UUCAAGCCUCCAAGCUGUG | 23 | UUCAAGCCUCCAAGCUGUG | 23 | CACAGCUUGGAGGCUUGAA | 669 |
| GCCUCCAAGCUGUGCCUUG | 24 | GCCUCCAAGCUGUGCCUUG | 24 | CAAGGCACAGCUUGGAGGC | 670 |
| AAGAAGAACUCCCUCGCCU | 25 | AAGAAGAACUCCCUCGCCU | 25 | AGGCGAGGGAGUUCUUCUU | 671 |
| CAAGCCUCCAAGCUGUGCC | 26 | CAAGCCUCCAAGCUGUGCC | 26 | GGCACAGCUUGGAGGCUUG | 672 |
| AGAAGAACUCCCUCGCCUC | 27 | AGAAGAACUCCCUCGCCUC | 27 | GAGGCGAGGGAGUUCUUCU | 673 |
| AGCCUCCAAGCUGUGCCUU | 28 | AGCCUCCAAGCUGUGCGUU | 28 | AAGGCACAGCUUGGAGGCU | 674 |
| ACUCGUGGUGGACUUCUCU | 29 | ACUCGUGGUGGACUUCUCU | 29 | AGAGAAGUCCACCACGAGU | 675 |
| UGUGCACUUCGCUUCACCU | 30 | UGUGCACUUCGCUUCACCU | 30 | AGGUGAAGCGAAGUGCACA | 676 |
| CCGUGUGCACUUCGCUUCA | 31 | CCGUGUGCACUUCGCUUCA | 31 | UGAAGCGAAGUGCACACGG | 677 |
| CGUGUGCACUUCGCUUCAC | 32 | CGUGUGCACUUCGCUUCAC | 32 | GUGAAGCGAAGUGCACACG | 678 |
| UCGCUUCACCUCUGCACGU | 33 | UCGCUUCACCUCUGCACGU | 33 | ACGUGCAGAGGUGAAGCGA | 679 |
| GUGUGCACUUCGCUUCACC | 34 | GUGUGCACUUCGCUUCACC | 34 | GGUGAAGCGAAGUGCACAC | 680 |
| UUCGCUUCACCUCUGCACG | 35 | UUCGCUUCACCUCUGCACG | 35 | CGUGCAGAGGUGAAGCGAA | 681 |
| ACUUCGCUUCACCUCUGCA | 36 | ACUUCGCUUCACCUCUGCA | 36 | UGCAGAGGUGAAGCGAAGU | 682 |
| CUUCGCUUCACCUCUGCAC | 37 | CUUCGCUUCACCUCUGCAC | 37 | GUGCAGAGGUGAAGCGAAG | 683 |
| CACUUCGCUUCACCUCUGC | 38 | CACUUCGCUUCACCUCUGC | 38 | GCAGAGGUGAAGCGAAGUG | 684 |
| CCUAUGGGAGUGGGCCUCA | 39 | CCUAUGGGAGUGGGCCUCA | 39 | UGAGGCCCACUCCCAUAGG | 685 |
| CGCACCUCUCUUUACGCGG | 40 | CGCACCUCUCUUUACGCGG | 40 | CCGCGUAAAGAGAGGUGCG | 686 |
| GUGCACUUCGCUUCACCUC | 41 | GUGCACUUCGCUUCACCUC | 41 | GAGGUGAAGCGAAGUGCAC | 687 |
| GACUCGUGGUGGACUUCUC | 42 | GACUCGUGGUGGACUUCUC | 42 | GAGAAGUCCACCACGAGUC | 688 |
| UCUAGACUCGUGGUGGACU | 43 | UCUAGACUCGUGGUGGACU | 43 | AGUCCACCACGAGUCUAGA | 689 |
| CUAGACUCGUGGUGGACUU | 44 | CUAGACUCGUGGUGGACUU | 44 | AAGUCCACCACGAGUCUAG | 690 |
| GCACUUCGCUUCACCUCUG | 45 | GCACUUCGCUUCACGUCUG | 45 | CAGAGGUGAAGCGAAGUGC | 691 |
| AGACUCGUGGUGGACUUCU | 46 | AGACUCGUGGUGGACUUCU | 46 | AGAAGUCCACCACGAGUCU | 692 |
| UGCACUUCGCUUCACCUCU | 47 | UGCACUUCGCUUCACCUCU | 47 | AGAGGUGAAGCGAAGUGCA | 693 |
| UAGACUCGUGGUGGACUUC | 48 | UAGACUCGUGGUGGACUUC | 48 | GAAGUCCACCACGAGUCUA | 694 |
| AGUCUAGACUGGUGGUGGA | 49 | AGUCUAGACUCGUGGUGGA | 49 | UCCACCACGAGUCUAGACU | 695 |
| GAGUCUAGACUCGUGGUGG | 50 | GAGUCUAGACUCGUGGUGG | 50 | CCACCACGAGUCUAGACUC | 696 |
| GUCUAGACUCGUGGUGGAC | 51 | GUCUAGACUCGUGGUGGAC | 51 | GUCCACCACGAGUCUAGAC | 697 |
| GUUCAAGCCUCCAAGCUGU | 52 | GUUCAAGCCUCCAAGCUGU | 52 | ACAGCUUGGAGGCUUGAAC | 698 |
| AAGCUGUGCCUUGGGUGGC | 53 | AAGCUGUGCCUUGGGUGGC | 53 | GCCACCCAAGGCACAGCUU | 699 |
| CUGUGCCUUGGGUGGCUUU | 54 | CUGUGCCUUGGGUGGCUUU | 54 | AAAGCCACCCAAGGCACAG | 700 |
| UGUUCAAGCCUCCAAGCUG | 55 | UGUUCAAGCCUCCAAGCUG | 55 | CAGCUUGGAGGCUUGAACA | 701 |
| CAAGCUGUGCCUUGGGUGG | 56 | CAAGCUGUGCCUUGGGUGG | 56 | CCACCCAAGGCACAGCUUG | 702 |
| CUCCAAGCUGUGCCUUGGG | 57 | CUCCAAGCUGUGCCUUGGG | 57 | CCCAAGGCACAGCUUGGAG | 703 |
| CCAAGCUGUGCCUUGGGUG | 58 | CCAAGCUGUGCCUUGGGUG | 58 | CACCCAAGGCACAGCUUGG | 704 |
| UCCAAGCUGUGCCUUGGGU | 59 | UCCAAGCUGUGCCUUGGGU | 59 | ACCCAAGGCACAGCUUGGA | 705 |
| CUAUGGGAGUGGGCCUCAG | 60 | CUAUGGGAGUGGGCCUCAG | 60 | CUGAGGCCCACUCCCAUAG | 706 |
| AGCUGUGCCUUGGGUGGCU | 61 | AGCUGUGCCUUGGGUGGCU | 61 | AGCCACCCAAGGCACAGCU | 707 |
| ACUGUUCAAGCCUCCAAGC | 62 | ACUGUUCAAGCCUCCAAGC | 62 | GCUUGGAGGCUUGAACAGU | 708 |
| AGAGUCUAGACUCGUGGUG | 63 | AGAGUCUAGACUCGUGGUG | 63 | CACCACGAGUCUAGACUCU | 709 |
| GCUGUGCCUUGGGUGGCUU | 64 | GCUGUGCCUUGGGUGGCUU | 64 | AAGCCACCCAAGGCACAGC | 710 |
| CUGUUCAAGCCUCCAAGCU | 65 | CUGUUCAAGCCUCCAAGCU | 65 | AGCUUGGAGGCUUGAACAG | 711 |
| GGUAUGUUGCCCGUUUGUC | 66 | GGUAUGUUGCCCGUUUGUC | 66 | GACAAACGGGCAACAUACC | 712 |
| UGGAUGUGUCUGCGGCGUU | 67 | UGGAUGUGUCUGCGGCGUU | 67 | AACGCCGCAGACACAUCCA | 713 |
| CUGCUGGUGGCUCCAGUUC | 68 | CUGCUGGUGGCUCCAGUUC | 68 | GAACUGGAGCCACCAGCAG | 714 |
| CCUGCUGGUGGCUCCAGUU | 69 | CCUGCUGGUGGCUCCAGUU | 69 | AACUGGAGCCACCAGCAGG | 715 |
| GUAUGUUGCCCGUUUGUCC | 70 | GUAUGUUGCCCGUUUGUCC | 70 | GGACAAACGGGCAACAUAC | 716 |
| UGGCUCAGUUUACUAGUGC | 71 | UGGCUCAGUUUACUAGUGC | 71 | GCACUAGUAAACUGAGCCA | 717 |
| CCGAUCCAUACUGCGGAAC | 72 | CCGAUCCAUACUGCGGAAC | 72 | GUUCCGCAGUAUGGAUCGG | 718 |
| CGAUCCAUACUGCGGAACU | 73 | CGAUCCAUACUGCGGAACU | 73 | AGUUCCGCAGUAUGGAUCG | 719 |
| AGGUAUGUUGCCCGUUUGU | 74 | AGGUAUGUUGCCCGUUUGU | 74 | ACAAACGGGCAACAUACCU | 720 |
| CAGAGUCUAGACUCGUGGU | 75 | CAGAGUCUAGACUCGUGGU | 75 | ACCACGAGUCUAGACUCUG | 721 |
| UGGACUUCUCUCAAUUUUC | 76 | UGGACUUCUCUCAAUUUUC | 76 | GAAAAUUGAGAGAAGUCCA | 722 |
| GGACUUCUCUCAAUUUUCU | 77 | GGACUUCUCUCAAUUUUCU | 77 | AGAAAAUUGAGAGAAGUCC | 723 |
| UGGUGGACUUCUCUCAAUU | 78 | UGGUGGACUUCUCUCAAUU | 78 | AAUUGAGAGAAGUCCACCA | 724 |
| AUGUUGCCCGUUUGUCCUC | 79 | AUGUUGCCCGUUUGUCCUC | 79 | GAGGACAAACGGGCAACAU | 725 |
| GACUUCUCUCAAUUUUCUA | 80 | GACUUCUCUCAAUUUUCUA | 80 | UAGAAAAUUGAGAGAAGUC | 726 |
| CUCCUCUGCCGAUCCAUAC | 81 | CUCCUCUGCCGAUCCAUAC | 81 | GUAUGGAUCGGCAGAGGAG | 727 |
| GUGGUGGACUUCUCUCAAU | 82 | GUGGUGGACUUCUCUCAAU | 82 | AUUGAGAGAAGUCCACCAC | 728 |
| UAUGUUGCCCGUUUGUCCU | 83 | UAUGUUGCCCGUUUGUCCU | 83 | AGGACAAACGGGCAACAUA | 729 |
| GUGGACUUCUCUCAAUUUU | 84 | GUGGACUUCUCUCAAUUUU | 84 | AAAAUUGAGAGAAGUCCAC | 730 |
| AACUUUUUCACCUCUGCCU | 85 | AACUUUUUCACCUCUGCCU | 85 | AGGCAGAGGUGAAAAAGUU | 731 |
| GAGUGUGGAUUCGCACUCC | 86 | GAGUGUGGAUUCGCACUCC | 86 | GGAGUGCGAAUCCACACUC | 732 |
| AUGUGUCUGCGGCGUUUUA | 87 | AUGUGUCUGCGGCGUUUUA | 87 | UAAAACGCCGCAGACACAU | 733 |
| GAUGUGUCUGCGGCGUUUU | 88 | GAUGUGUCUGCGGCGUUUU | 88 | AAAACGCCGCAGACACAUC | 734 |
| GGUGGACUUCUCUCAAUUU | 89 | GGUGGACUUCUCUCAAUUU | 89 | AAAUUGAGAGAAGUCCACC | 735 |
| GUGUCUGCGGCGUUUUAUC | 90 | GUGUCUGCGGCGUUUUAUC | 90 | GAUAAAACGCCGCAGACAC | 736 |
| UAGAAGAAGAACUCCCUCG | 91 | UAGAAGAAGAACUCCCUCG | 91 | CGAGGGAGUUCUUCUUCUA | 737 |
| AGAAGAAGAACUCCCUCGC | 92 | AGAAGAAGAACUCCCUCGC | 92 | GCGAGGGAGUUCUUCUUCU | 738 |
| UGUCUGCGGCGUUUUAUCA | 93 | UGUCUGCGGCGUUUUAUCA | 93 | UGAUAAAACGCCGCAGACA | 739 |
| ACUUUUUCACCUCUGCCUA | 94 | ACUUUUUCACCUCUGCCUA | 94 | UAGGCAGAGGUGAAAAAGU | 740 |
| CCUGCUCGUGUUACAGGCG | 95 | CCUGCUCGUGUUACAGGCG | 95 | CGCCUGUAACACGAGCAGG | 741 |
| GUCUGCGGCGUUUUAUCAU | 96 | GUCUGCGGCGUUUUAUCAU | 96 | AUGAUAAAACGCCGCAGAC | 742 |
| ACUUCUCUCAAUUUUCUAG | 97 | ACUUCUCUCAAUUUUCUAG | 97 | CUAGAAAAUUGAGAGAAGU | 743 |
| CCUAGAAGAAGAACUCCCU | 98 | CCUAGAAGAAGAACUCCCU | 98 | AGGGAGUUCUUCUUCUAGG | 744 |
| GGAGUGUGGAUUCGCACUC | 99 | GGAGUGUGGAUUCGCACUC | 99 | GAGUGCGAAUCCACACUCC | 745 |
| UGUGUCUGCGGCGUUUUAU | 100 | UGUGUCUGCGGCGUUUUAU | 100 | AUAAAACGCCGCAGACACA | 746 |
| CUAGAAGAAGAACUCCCUC | 101 | CUAGAAGAAGAACUCCCUC | 101 | GAGGGAGUUCUUCUUCUAG | 747 |
| CCCUAGAAGAAGAACUCCC | 102 | CCCUAGAAGAAGAACUCCC | 102 | GGGAGUUCUUCUUCUAGGG | 748 |
| CUGCUCGUGUUACAGGCGG | 103 | CUGCUCGUGUUACAGGCGG | 103 | CCGCCUGUAACACGAGCAG | 749 |
| GGAUGUGUCUGCGGCGUUU | 104 | GGAUGUGUCUGCGGCGUUU | 104 | AAACGCCGCAGACACAUCC | 750 |
| CCCCUAGAAGAAGAACUCC | 105 | CCCCUAGAAGAAGAACUCC | 105 | GGAGUUCUUCUUCUAGGGG | 751 |
| CGUGGUGGACUUCUCUCAA | 106 | CGUGGUGGACUUCUCUCAA | 106 | UUGAGAGAAGUCCACCACG | 752 |
| GGACCCCUGCUCGUGUUAC | 107 | GGACCCCUGCUCGUGUUAC | 107 | GUAACACGAGCAGGGGUCC | 753 |
| UGUUGCCCGUUUGUCCUCU | 108 | UGUUGCCCGUUUGUCCUCU | 108 | AGAGGACAAACGGGCAACA | 754 |
| CCCUGCUCGUGUUACAGGC | 109 | CCCUGCUCGUGUUACAGGC | 109 | GCCUGUAACACGAGCAGGG | 755 |
| GACCCCUGCUCGUGUUACA | 110 | GACCCCUGCUCGUGUUACA | 110 | UGUAACACGAGCAGGGGUC | 756 |
| CCCCUGCUCGUGUUACAGG | 111 | CCCCUGCUCGUGUUACAGG | 111 | CCUGUAACACGAGCAGGGG | 757 |
| UUCUCUCAAUUUUCUAGGG | 112 | UUCUCUCAAUUUUCUAGGG | 112 | CCCUAGAAAAUUGAGAGAA | 758 |
| ACCCCUGCUCGUGUUACAG | 113 | ACCCCUGCUCGUGUUACAG | 113 | CUGUAACACGAGCAGGGGU | 759 |
| CUUCUCUCAAUUUUCUAGG | 114 | CUUCUCUCAAUUUUCUAGG | 114 | CCUAGAAAAUUGAGAGAAG | 760 |
| AAGGUAUGUUGCCCGUUUG | 115 | AAGGUAUGUUGCCCGUUUG | 115 | CAAACGGGCAACAUACCUU | 761 |
| GCCGAUCCAUACUGCGGAA | 116 | GCCGAUCCAUACUGCGGAA | 116 | UUCCGCAGUAUGGAUCGGC | 762 |
| GAUCCAUACUGCGGAACUC | 117 | GAUCCAUACUGCGGAACUC | 117 | GAGUUCCGCAGUAUGGAUC | 763 |
| UCCAUACUGCGGAACUCCU | 118 | UCCAUACUGCGGAACUCCU | 118 | AGGAGUUCCGCAGUAUGGA | 764 |
| UCUCUCAAUUUUCUAGGGG | 119 | UCUCUCAAUUUUCUAGGGG | 119 | CCCCUAGAAAAUUGAGAGA | 765 |
| AUCCAUACUGCGGAACUCC | 120 | AUCCAUACUGCGGAACUCC | 120 | GGAGUUCCGCAGUAUGGAU | 766 |
| UGCCGAUCCAUACUGCGGA | 121 | UGCCGAUCCAUACUGCGGA | 121 | UCCGCAGUAUGGAUCGGCA | 767 |
| AACUCCCUCGCCUCGCAGA | 122 | AACUCCCUCGCCUCGCAGA | 122 | UCUGCGAGGCGAGGGAGUU | 768 |
| CGUCGCAGAAGAUCUCAAU | 123 | CGUCGCAGAAGAUCUCAAU | 123 | AUUGAGAUCUUCUGCGACG | 769 |
| CUGCCGAUCCAUACUGCGG | 124 | CUGCCGAUCCAUACUGCGG | 124 | CCGCAGUAUGGAUCGGCAG | 770 |
| GAACUCCCUCGCCUCGCAG | 125 | GAACUCCCUCGCCUCGCAG | 125 | CUGCGAGGCGAGGGAGUUC | 771 |
| GUCGCAGAAGAUCUCAAUC | 126 | GUCGCAGAAGAUCUCAAUC | 126 | GAUUGAGAUCUUCUGCGAC | 772 |
| AGGACCCCUGCUCGUGUUA | 127 | AGGACCCCUGCUCGUGUUA | 127 | UAACACGAGCAGGGGUCCU | 773 |
| UCGCAGAAGAUCUCAAUCU | 128 | UCGCAGAAGAUCUCAAUCU | 128 | AGAUUGAGAUCUUCUGCGA | 774 |
| AGAACUCCCUCGCCUCGCA | 129 | AGAACUCCCUCGCCUCGCA | 129 | UGCGAGGCGAGGGAGUUCU | 775 |
| UCUGCCGAUCCAUACUGCG | 130 | UCUGCCGAUCCAUACUGCG | 130 | CGCAGUAUGGAUCGGCAGA | 776 |
| CGCGUCGCAGAAGAUCUCA | 131 | CGCGUCGCAGAAGAUCUCA | 131 | UGAGAUCUUCUGCGACGCG | 777 |
| CCUCUGCCGAUCCAUACUG | 132 | CCUCUGCCGAUCCAUACUG | 132 | CAGUAUGGAUCGGCAGAGG | 778 |
| GCGUCGCAGAAGAUCUCAA | 133 | GCGUCGCAGAAGAUCUCAA | 133 | UUGAGAUCUUCUGCGACGC | 779 |
| CUCUGCCGAUCCAUACUGC | 134 | CUCUGCCGAUCCAUACUGC | 134 | GCAGUAUGGAUCGGCAGAG | 780 |
| CGCAGAAGAUCUCAAUCUC | 135 | CGCAGAAGAUCUCAAUCUC | 135 | GAGAUUGAGAUCUUCUGCG | 781 |
| UCCUCUGCCGAUCCAUACU | 136 | UCCUCUGCCGAUCCAUACU | 136 | AGUAUGGAUCGGCAGAGGA | 782 |
| UCCCCUAGAAGAAGAACUC | 137 | UCCCCUAGAAGAAGAACUC | 137 | GAGUUCUUCUUCUAGGGGA | 783 |
| CCGCGUCGCAGAAGAUCUC | 138 | CCGCGUCGCAGAAGAUCUC | 138 | GAGAUCUUCUGCGACGCGG | 784 |
| CCAAGUGUUUGCUGACGCA | 139 | CCAAGUGUUUGCUGACGCA | 139 | UGCGUCAGCAAACACUUGG | 785 |
| UGCCAAGUGUUUGCUGACG | 140 | UGCCAAGUGUUUGCUGACG | 140 | CGUCAGCAAACACUUGGCA | 786 |
| AGGAGGCUGUAGGCAUAAA | 141 | AGGAGGCUGUAGGCAUAAA | 141 | UUUAUGCCUACAGCCUCCU | 787 |
| UAGGAGGCUGUAGGCAUAA | 142 | UAGGAGGCUGUAGGCAUAA | 142 | UUAUGCCUACAGCCUCCUA | 788 |
| GCCAAGUGUUUGCUGACGC | 143 | GCCAAGUGUUUGCUGACGC | 143 | GCGUCAGCAAACACUUGGC | 789 |
| CUCCCUCGCCUCGCAGACG | 144 | CUCCCUCGCCUCGCAGACG | 144 | CGUCUGCGAGGCGAGGGAG | 790 |
| UUGCUGACGCAACCCCCAC | 145 | UUGCUGACGCAACCCCCAC | 145 | GUGGGGGUUGCGUCAGCAA | 791 |
| UCCCGUCGGCGCUGAAUCC | 146 | UCCCGUCGGCGCUGAAUCC | 146 | GGAUUCAGCGCCGACGGGA | 792 |
| GCAACUUUUUCACCUCUGC | 147 | GCAACUUUUUCACCUCUGC | 147 | GCAGAGGUGAAAAAGUUGC | 793 |
| GGUCCCCUAGAAGAAGAAC | 148 | GGUCCCCUAGAAGAAGAAC | 148 | GUUCUUCUUCUAGGGGACC | 794 |
| GCAGGUCCCCUAGAAGAAG | 149 | GCAGGUCCCCUAGAAGAAG | 149 | CUUCUUCUAGGGGACCUGC | 795 |
| ACUCCCUCGCCUCGCAGAC | 150 | ACUCCCUCGCCUCGGAGAC | 150 | GUCUGCGAGGCGAGGGAGU | 796 |
| CGUCCCGUCGGCGCUGAAU | 151 | CGUCCCGUCGGCGCUGAAU | 151 | AUUCAGCGCCGACGGGACG | 797 |
| CAGGUCCCCUAGAAGAAGA | 152 | CAGGUCCCCUAGAAGAAGA | 152 | UCUUCUUCUAGGGGACCUG | 798 |
| AGGUCCCCUAGAAGAAGAA | 153 | AGGUCCCCUAGAAGAAGAA | 153 | UUCUUCUUCUAGGGGACCU | 799 |
| UACGUCCCGUCGGCGCUGA | 154 | UACGUCCCGUCGGCGCUGA | 154 | UCAGCGCCGACGGGACGUA | 800 |
| CAAGUGUUUGCUGACGCAA | 155 | CAAGUGUUUGCUGACGCAA | 155 | UUGCGUCAGCAAACACUUG | 801 |
| ACGUCCCGUCGGCGCUGAA | 156 | ACGUCCCGUCGGCGCUGAA | 156 | UUCAGCGCCGACGGGACGU | 802 |
| CAAGGUAUGUUGCCCGUUU | 157 | CAAGGUAUGUUGCCCGUUU | 157 | AAACGGGCAACAUACCUUG | 803 |
| UUUGCUGACGCAACCCCCA | 158 | UUUGCUGACGCAACCCCCA | 158 | UGGGGGUUGCGUCAGCAAA | 804 |
| CAACUUUUUCACCUCUGCC | 159 | CAACUUUUUCACCUCUGCC | 159 | GGCAGAGGUGAAAAAGUUG | 805 |
| UCCUAGGACCCCUGCUCGU | 160 | UCCUAGGACCCCUGCUCGU | 160 | ACGAGCAGGGGUCCUAGGA | 806 |
| GUCCCGUCGGCGCUGAAUC | 161 | GUCCCGUCGGCGCUGAAUC | 161 | GAUUCAGCGCCGACGGGAC | 807 |
| AAGUGUUUGCUGACGCAAC | 162 | AAGUGUUUGCUGACGCAAC | 162 | GUUGCGUCAGCAAACACUU | 808 |
| CCUAGGACCCCUGCUCGUG | 163 | CCUAGGACCCCUGCUCGUG | 163 | CACGAGCAGGGGUCCUAGG | 809 |
| GUGUUUGCUGACGCAACCC | 164 | GUGUUUGCUGACGCAACCC | 164 | GGGUUGCGUCAGCAAACAC | 810 |
| AGUGUUUGCUGACGCAACC | 165 | AGUGUUUGCUGACGCAACC | 165 | GGUUGCGUCAGCAAACACU | 811 |
| CUAGGACCCCUGCUCGUGU | 166 | CUAGGACCCCUGCUCGUGU | 166 | ACACGAGCAGGGGUCCUAG | 812 |
| GUCCCCUAGAAGAAGAACU | 167 | GUCCCCUAGAAGAAGAACU | 167 | AGUUCUUCUUCUAGGGGAC | 813 |
| GUUUGCUGACGCAACCCCC | 168 | GUUUGCUGACGCAACCCCC | 168 | GGGGGUUGCGUCAGCAAAC | 814 |
| UGUUUGCUGACGCAACCCC | 169 | UGUUUGCUGACGCAACCCC | 169 | GGGGUUGCGUCAGCAAACA | 815 |
| UAGGACCCCUGCUCGUGUU | 170 | UAGGACCCCUGCUCGUGUU | 170 | AACACGAGCAGGGGUCCUA | 816 |
| UGCAACUUUUUCACCUCUG | 171 | UGCAACUUUUUCACCUCUG | 171 | CAGAGGUGAAAAAGUUGCA | 817 |
| CUGACGCAACCCCCACUGG | 172 | CUGACGCAACCCCCACUGG | 172 | CCAGUGGGGGUUGCGUCAG | 818 |
| AUGCAACUUUUUCACCUCU | 173 | AUGCAACUUUUUCACCUCU | 173 | AGAGGUGAAAAAGUUGCAU | 819 |
| UGCUGACGCAACCCCCACU | 174 | UGCUGACGCAACCCCCACU | 174 | AGUGGGGGUUGCGUCAGCA | 820 |
| GCUGACGCAACCCCCACUG | 175 | GCUGACGCAACCCCCACUG | 175 | CAGUGGGGGUUGCGUCAGC | 821 |
| GGGCGCACCUCUCUUUACG | 176 | GGGCGCACCUCUCUUUACG | 176 | CGUAAAGAGAGGUGCGCCC | 822 |
| GGCGCACCUCUCUUUACGC | 177 | GGCGCACCUCUCUUUACGC | 177 | GCGUAAAGAGAGGUGCGCC | 823 |
| GGCCAAAAUUCGCAGUCCC | 178 | GGCCAAAAUUCGCAGUCCC | 178 | GGGACUGCGAAUUUUGGCC | 824 |
| UGGCCAAAAUUCGCAGUCC | 179 | UGGCCAAAAUUCGCAGUCC | 179 | GGACUGCGAAUUUUGGCCA | 825 |
| UUACAGGCGGGGUUUUUCU | 180 | UUACAGGCGGGGUUUUUCU | 180 | AGAAAAACCCCGCCUGUAA | 826 |
| GGGGCGCACCUCUCUUUAC | 181 | GGGGCGCACCUCUCUUUAC | 181 | GUAAAGAGAGGUGCGCCCC | 827 |
| CGGGGCGCACCUCUCUUUA | 182 | CGGGGCGCACCUCUCUUUA | 182 | UAAAGAGAGGUGCGCCCCG | 828 |
| GUUACAGGCGGGGUUUUUC | 183 | GUUACAGGCGGGGUUUUUC | 183 | GAAAPACCCCGCCUGUAAC | 829 |
| CACCUCUGCCUAAUCAUCU | 184 | CACCUCUGCCUAAUCAUCU | 184 | AGAUGAUUAGGCAGAGGUG | 830 |
| UUUCACCUCUGCCUAAUCA | 185 | UUUCACCUCUGCCUAAUCA | 185 | UGAUUAGGCAGAGGUGAAA | 831 |
| UUCACCUCUGCCUAAUCAU | 186 | UUCACCUCUGCCUAAUCAU | 186 | AUGAUUAGGCAGAGGUGAA | 832 |
| GCGCACCUCUCUUUACGCG | 187 | GCGCACCUCUCUUUACGCG | 187 | CGCGUAAAGAGAGGUGCGC | 833 |
| CGUAGGGCUUUCCCCCACU | 188 | CGUAGGGCUUUCCGCCACU | 188 | AGUGGGGGAAAGCCCUACG | 834 |
| ACGGGGCGCACCUCUCUUU | 189 | ACGGGGCGCACCUCUCUUU | 189 | AAAGAGAGGUGCGCCCCGU | 835 |
| AUAAGAGGACUCUUGGACU | 190 | AUAAGAGGACUCUUGGACU | 190 | AGUCCAAGAGUCCUCUUAU | 836 |
| UCACCUCUGCCUAAUCAUC | 191 | UCACCUCUGCCUAAUCAUC | 191 | GAUGAUUAGGCAGAGGUGA | 837 |
| GUAGGGCUUUCCCCCACUG | 192 | GUAGGGCUUUCCCCCACUG | 192 | CAGUGGGGGAAAGCCCUAC | 838 |
| UAGGGCUUUCCCCCACUGU | 193 | UAGGGCUUUCCCCCACUGU | 193 | ACAGUGGGGGAAAGCCCUA | 839 |
| UUUUCACCUCUGCCUAAUC | 194 | UUUUCACCUCUGCCUAAUC | 194 | GAUUAGGCAGAGGUGAAAA | 840 |
| UUUUUCACCUCUGCCUAAU | 195 | UUUUUCACCUCUGCCUAAU | 195 | AUUAGGCAGAGGUGAAAAA | 841 |
| CCAUUUGUUCAGUGGUUCG | 196 | CCAUUUGUUCAGUGGUUCG | 196 | CGAACCACUGAACAAAUGG | 842 |
| GUGUUACAGGCGGGGUUUU | 197 | GUGUUACAGGCGGGGUUUU | 197 | AAAACCCCGCCUGUAACAC | 843 |
| CGUGUUACAGGCGGGGUUU | 198 | CGUGUUACAGGCGGGGUUU | 198 | AAACCCCGCCUGUAACACG | 844 |
| CCACGGGGCGCACCUCUCU | 199 | CCACGGGGCGCACCUCUCU | 199 | AGAGAGGUGCGCCCCGUGG | 845 |
| AGGCAGGUCCCCUAGAAGA | 200 | AGGCAGGUCCCCUAGAAGA | 200 | UCUUCUAGGGGACCUGCCU | 846 |
| CUCUCAAUUUUCUAGGGGG | 201 | CUCUCAAUUUUCUAGGGGG | 201 | CCCCCUAGAAAAUUGAGAG | 847 |
| GAGGCAGGUCCCCUAGAAG | 202 | GAGGCAGGUCCCCUAGAAG | 202 | CUUCUAGGGGACCUGCCUC | 848 |
| UGUAUUCCCAUCCCAUCAU | 203 | UGUAUUCCCAUCCCAUCAU | 203 | AUGAUGGGAUGGGAAUACA | 849 |
| GUAUUCCCAUCCCAUCAUC | 204 | GUAUUCCCAUCCCAUCAUC | 204 | GAUGAUGGGAUGGGAAUAC | 850 |
| CUCGUGUUACAGGCGGGGU | 205 | CUCGUGUUACAGGCGGGGU | 205 | ACCCCGCCUGUAACACGAG | 851 |
| UCGUGUUACAGGCGGGGUU | 206 | UCGUGUUACAGGCGGGGUU | 206 | AACCCCGCCUGUAACACGA | 852 |
| GUUGCCCGUUUGUCCUCUA | 207 | GUUGCCCGUUUGUCCUCUA | 207 | UAGAGGACAAACGGGCAAC | 853 |
| GCCAUUUGUUCAGUGGUUC | 208 | GCCAUUUGUUCAGUGGUUC | 208 | GAACCACUGAACAAAUGGC | 854 |
| CACGGGGCGCACCUCUCUU | 209 | CACGGGGCGCACCUCUCUU | 209 | AAGAGAGGUGCGCCCCGUG | 855 |
| UGUUACAGGCGGGGUUUUU | 210 | UGUUACAGGCGGGGUUUUU | 210 | AAAAACCCCGCCUGUAACA | 856 |
| GGCAGGUCCCCUAGAAGAA | 211 | GGCAGGUCCCCUAGAAGAA | 211 | UUCUUCUAGGGGACCUGCC | 857 |
| CUAUGCCUCAUCUUCUUGU | 212 | CUAUGCCUCAUCUUCUUGU | 212 | ACAAGAAGAUGAGGCAUAG | 858 |
| CUCAAUCGCCGCGUCGCAG | 213 | CUCAAUCGCCGCGUCGCAG | 213 | CUGCGACGCGGCGAUUGAG | 859 |
| UCAAUCGCCGCGUCGCAGA | 214 | UCAAUCGCCGCGUCGCAGA | 214 | UCUGCGACGCGGCGAUUGA | 860 |
| CACCAUAUUCUUGGGAACA | 215 | CACCAUAUUCUUGGGAACA | 215 | UGUUCCCAAGAAUAUGGUG | 861 |
| UCACCAUAUUCUUGGGAAC | 216 | UCACCAUAUUCUUGGGAAC | 216 | GUUCCCAAGAAUAUGGUGA | 862 |
| ACCAUAUUCUUGGGAACAA | 217 | ACCAUAUUCUUGGGAACAA | 217 | UUGUUCCCAAGAAUAUGGU | 863 |
| GCUAUGCCUCAUCUUCUUG | 218 | GCUAUGCCUCAUCUUCUUG | 218 | CAAGAAGAUGAGGCAUAGC | 864 |
| GCCGCGUCGCAGAAGAUCU | 219 | GCCGCGUCGCAGAAGAUCU | 219 | AGAUCUUCUGCGACGCGGC | 865 |
| AAUCGCCGCGUCGCAGAAG | 220 | AAUCGCCGCGUCGCAGAAG | 220 | CUUCUGCGACGCGGCGAUU | 866 |
| CGCCGCGUCGCAGAAGAUC | 221 | CGCCGCGUCGCAGAAGAUC | 221 | GAUCUUCUGCGACGCGGCG | 867 |
| GGCUCAGUUUACUAGUGCC | 222 | GGCUCAGUUUACUAGUGCC | 222 | GGCACUAGUAAACUGAGCC | 868 |
| AUCGCCGCGUCGCAGAAGA | 223 | AUCGCCGCGUCGCAGAAGA | 223 | UCUUCUGCGACGCGGCGAU | 869 |
| AGUGUGGAUUCGCACUCCU | 224 | AGUGUGGAUUCGCACUCCU | 224 | AGGAGUGCGAACCACACU | 870 |
| UCGCCGCGUCGCAGAAGAU | 225 | UCGCCGCGUCGCAGAAGAU | 225 | AUCUUCUGCGACGCGGCGA | 871 |
| CUCAUCUUCUUGUUGGUUC | 226 | CUCAUCUUCUUGUUGGUUC | 226 | GAACCAACAAGAAGAUGAG | 872 |
| CAUAUUCUUGGGAACAAGA | 227 | CAUAUUCUUGGGAACAAGA | 227 | UCUUGUUCCCAAGAAUAUG | 873 |
| AUGCCUCAUCUUCUUGUUG | 228 | AUGCCUCAUCUUCUUGUUG | 228 | CAACAAGAAGAUGAGGCAU | 874 |
| CUCCCCGUCUGUGCCUUCU | 229 | CUCCCCGUCUGUGCCUUCU | 229 | AGAAGGCACAGACGGGGAG | 875 |
| GCCUCAUCUUCUUGUUGGU | 230 | GCCUCAUCUUCUUGUUGGU | 230 | ACCAACAAGAAGAUGAGGC | 876 |
| UAUGCCUCAUCUUCUUGUU | 231 | UAUGCGUCAUCUUCUUGUU | 231 | AACAAGAAGAUGAGGCAUA | 877 |
| UCAUCUUCUUGUUGGUUCU | 232 | UCAUCUUCUUGUUGGUUCU | 232 | AGAACCAACAAGAAGAUGA | 878 |
| CCUCAUCUUCUUGUUGGUU | 233 | CCUCAUCUUCUUGUUGGUU | 233 | AACCAACAAGAAGAUGAGG | 879 |
| UCCCCGUCUGUGCCUUCUC | 234 | UCCCCGUCUGUGCCUUCUC | 234 | GAGAAGGCACAGACGGGGA | 880 |
| UGCCUCAUCUUCUUGUUGG | 235 | UGCCUCAUCUUCUUGUUGG | 235 | CCAACAAGAAGAUGAGGCA | 881 |
| UCUCAAUCGCCGCGUCGCA | 236 | UCUCAAUCGCCGCGUCGCA | 236 | UGCGACGCGGCGAUUGAGA | 882 |
| UCUUGUUGGUUCUUCUGGA | 237 | UCUUGUUGGUUCUUCUGGA | 237 | UCCAGAAGAACCAACAAGA | 883 |
| GGGUCACCAUAUUCUUGGG | 238 | GGGUCACCAUAUUCUUGGG | 238 | CCCAAGAAUAUGGUGACCC | 884 |
| UAUCGCUGGAUGUGUCUGC | 239 | UAUCGCUGGAUGUGUCUGC | 239 | GCAGACACAUCCAGCGAUA | 885 |
| CAUCUUCUUGUUGGUUCUU | 240 | CAUCUUCUUGUUGGUUCUU | 240 | AAGAACCAACAAGAAGAUG | 886 |
| GUCACCAUAUUCUUGGGAA | 241 | GUCACCAUAUUCUUGGGAA | 241 | UUCCCAAGAAUAUGGUGAC | 887 |
| CCAUAUUCUUGGGAACAAG | 242 | CCAUAUUCUUGGGAACAAG | 242 | CUUGUUCCCAAGAAUAUGG | 888 |
| GGUCACCAUAUUCUUGGGA | 243 | GGUCACCAUAUUCUUGGGA | 243 | UCCCAAGAAUAUGGUGACC | 889 |
| CUUCUUGUUGGUUCUUCUG | 244 | CUUCUUGUUGGUUCUUCUG | 244 | CAGAAGAACCAACAAGAAG | 890 |
| UUCUUGUUGGUUCUUCUGG | 245 | UUCUUGUUGGUUCUUCUGG | 245 | CCAGAAGAACCAACAAGAA | 891 |
| CAUGCAACUUUUUCACCUC | 246 | CAUGCAACUUUUUCACCUC | 246 | GAGGUGAAAAAGUUGCAUG | 892 |
| AUCGCUGGAUGUGUCUGCG | 247 | AUCGCUGGAUGUGUCUGCG | 247 | CGCAGACACAUCCAGCGAU | 893 |
| CAAUCGCCGGGUCGCAGAA | 248 | CAAUCGCCGCGUCGCAGAA | 248 | UUCUGCGACGCGGCGAUUG | 894 |
| UAGUGCCAUUUGUUCAGUG | 249 | UAGUGCCAUUUGUUCAGUG | 249 | CACUGAACAAAUGGCACUA | 895 |
| UCGCUGGAUGUGUCUGCGG | 250 | UCGCUGGAUGUGUCUGCGG | 250 | CGGGAGACACAUCCAGCGA | 896 |
| GUUUACUAGUGCCAUUUGU | 251 | GUUUACUAGUGCCAUUUGU | 251 | ACAAAUGGCACUAGUAAAC | 897 |
| CAGUUUACUAGUGCCAUUU | 252 | CAGUUUACUAGUGCCAUUU | 252 | AAAUGGCACUAGUAAACUG | 898 |
| CGCUGGAUGUGUCUGCGGC | 253 | CGCUGGAUGUGUCUGCGGC | 253 | GCCGCAGACACAUCCAGCG | 899 |
| UCUUCUUGUUGGUUCUUCU | 254 | UCUUCUUGUUGGUUCUUCU | 254 | AGAAGAACCAACAAGAAGA | 900 |
| CUAGUGCCAUUUGUUCAGU | 255 | CUAGUGCCAUUUGUUCAGU | 255 | ACUGAACAAAUGGCACUAG | 901 |
| AGUUUACUAGUGCCAUUUG | 256 | AGUUUACUAGUGCCAUUUG | 256 | CAAAUGGCACUAGUAAACU | 902 |
| GCUCGUGUUACAGGCGGGG | 257 | GCUCGUGUUACAGGCGGGG | 257 | CCCCGCCUGUAACACGAGC | 903 |
| CUUUUUCACCUCUGCCUAA | 258 | CUUUUUCACCUCUGCCUAA | 258 | UUAGGCAGAGGUGAAAAAG | 904 |
| ACUAGUGCCAUUUGUUCAG | 259 | ACUAGUGCCAUUUGUUCAG | 259 | CUGAACAAAUGGCACUAGU | 905 |
| AUCUUCUUGUUGGUUCUUC | 260 | AUCUUCUUGUUGGUUCUUC | 260 | GAAGAACCAACAAGAAGAU | 906 |
| UGCUCGUGUUACAGGCGGG | 261 | UGCUCGUGUUACAGGCGGG | 261 | CCCGCCUGUAACACGAGCA | 907 |
| UGAAUCCCGCGGACGACCC | 262 | UGAAUCCCGCGGACGACCC | 262 | GGGUCGUCCGCGGGAUUCA | 908 |
| AGUGCCAUUUGUUCAGUGG | 263 | AGUGCCAUUUGUUCAGUGG | 263 | CCACUGAACAAAUGGCACU | 909 |
| GCUCAGUUUACUAGUGCCA | 264 | GCUCAGUUUACUAGUGCCA | 264 | UGGGACUAGUAAACUGAGC | 910 |
| UUUACUAGUGCCAUUUGUU | 265 | UUUACUAGUGCCAUUUGUU | 265 | AACAAAUGGCACUAGUAAA | 911 |
| UACUAGUGCCAUUUGUUCA | 266 | UACUAGUGCCAUUUGUUCA | 266 | UGAACAAAUGGCACUAGUA | 912 |
| UCAGUUUACUAGUGCCAUU | 267 | UCAGUUUACUAGUGCCAUU | 267 | AAUGGCACUAGUPAACUGA | 913 |
| UUACUAGUGCCAUUUGUUC | 268 | UUACUAGUGCCAUUUGUUC | 268 | GAACAAAUGGCACUAGUAA | 914 |
| CUCAGUUUACUAGUGCCAU | 269 | CUCAGUUUACUAGUGCCAU | 269 | AUGGCACUAGUAAACUGAG | 915 |
| UCUCAAUUUUCUAGGGGGA | 270 | UCUCAAUUUUCUAGGGGGA | 270 | UCCCCCUAGAAAAUUGAGA | 916 |
| CUGAAUCCCGCGGACGACC | 271 | CUGAAUCCCGCGGACGACC | 271 | GGUCGUCCGCGGGAUUCAG | 917 |
| GCUGGAUGUGUCUGCGGCG | 272 | GCUGGAUGUGUCUGCGGCG | 272 | CGCCGCAGACACAUCCAGC | 918 |
| GCUGAAUCCCGCGGACGAC | 273 | GCUGAAUCCCGCGGACGAC | 273 | GUCGUCCGCGGGAUUCAGC | 919 |
| CUGGAUGUGUCUGCGGCGU | 274 | CUGGAUGUGUCUGCGGCGU | 274 | ACGCCGCAGACACAUCCAG | 920 |
| UGUGCUGCCAACUGGAUCC | 275 | UGUGCUGCCAACUGGAUCC | 275 | GGAUCCAGUUGGCAGCACA | 921 |
| GUGCCAUUUGUUCAGUGGU | 276 | GUGCCAUUUGUUCAGUGGU | 276 | ACCACUGAACAAAUGGCAC | 922 |
| UGCCAUUUGUUCAGUGGUU | 277 | UGCCAUUUGUUCAGUGGUU | 277 | AACCACUGAACAAAUGGCA | 923 |
| CCAUGCAACUUUUUCACCU | 278 | CCAUGCAACUUUUUCACCU | 278 | AGGUGAAAAAGUUGCAUGG | 924 |
| GUGCUGCCAACUGGAUCCU | 279 | GUGCUGCCAACUGGAUCCU | 279 | AGGAUCCAGUUGGCAGCAC | 925 |
| CAUGGAGACCACCGUGAAC | 280 | CAUGGAGACCACCGUGAAC | 280 | GUUCACGGUGGUCUCCAUG | 926 |
| UACAGGCGGGGUUUUUCUU | 281 | UACAGGCGGGGUUUUUCUU | 281 | AAGAAAAACCCCGCCUGUA | 927 |
| GCGCUGAAUCCCGCGGACG | 282 | GCGCUGAAUCCCGCGGACG | 282 | CGUCCGCGGGAUUCAGCGC | 928 |
| CUUGUUGGUUCUUCUGGAC | 283 | CUUGUUGGUUCUUCUGGAC | 283 | GUCCAGAAGAACCAACAAG | 929 |
| UUGUUGGUUCUUCUGGACU | 284 | UUGUUGGUUCUUCUGGACU | 284 | AGUCCAGAAGAACCAACAA | 930 |
| CGCUGAAUCCCGCGGACGA | 285 | CGCUGAAUCCCGCGGACGA | 285 | UCGUCCGCGGGAUUCAGCG | 931 |
| GCAUGGAGACCACCGUGAA | 286 | GCAUGGAGACCACCGUGAA | 286 | UUCACGGUGGUCUCCAUGC | 932 |
| ACAGGCGGGGUUUUUCUUG | 287 | ACAGGCGGGGUUUUUCUUG | 287 | CAAGAAAAACCCCGCCUGU | 933 |
| ACCACGGGGCGCACCUCUC | 288 | ACCACGGGGCGCACCUCUC | 288 | GAGAGGUGCGCCCCGUGGU | 934 |
| UGUUGGUUCUUCUGGACUA | 289 | UGUUGGUUCUUCUGGACUA | 289 | UAGUCCAGAAGAACCAACA | 935 |
| CGGCGCUGAAUCCCGCGGA | 290 | CGGCGCUGAAUCCCGCGGA | 290 | UCCGCGGGAUUCAGCGCCG | 936 |
| GGGGUUUUUCUUGUUGACA | 291 | GGGGUUUUUCUUGUUGACA | 291 | UGUCAACAAGAAAAACCCC | 937 |
| AUGGAGACCACCGUGAACG | 292 | AUGGAGACCACCGUGAACG | 292 | CGUUCACGGUGGUCUCCAU | 938 |
| UCGCCAACUUACAAGGCCU | 293 | UCGCCAACUUACAAGGCCU | 293 | AGGCCUUGUAAGUUGGCGA | 939 |
| CCGUCGGCGCUGAAUCCCG | 294 | CCGUCGGCGCUGAAUCCCG | 294 | CGGGAUUCAGCGCCGACGG | 940 |
| CAGGCGGGGUUUUUCUUGU | 295 | CAGGCGGGGUUUUUCUUGU | 295 | ACAAGAAAAACCCCGCCUG | 941 |
| GGCGCUGAAUCCCGCGGAC | 296 | GGCGCUGAAUCCCGCGGAC | 296 | GUCCGCGGGAUUCAGCGCC | 942 |
| CAGCACCAUGCAACUUUUU | 297 | CAGCACCAUGCAACUUUUU | 297 | AAAAAGUUGCAUGGUGCUG | 943 |
| CCAGCACCAUGCAACUUUU | 298 | CCAGCACCAUGCAACUUUU | 298 | AAAAGUUGCAUGGUGCUGG | 944 |
| CGUCGGCGCUGAAUCCCGC | 299 | CGUCGGCGCUGAAUCCCGC | 299 | GCGGGAUUCAGCGCCGACG | 945 |
| GGGUUUUUCUUGUUGACAA | 300 | GGGUUUUUCUUGUUGACAA | 300 | UUGUCAACAAGAAAAACCC | 946 |
| CCCGUCGGCGCUGAAUCCC | 301 | CCCGUCGGCGCUGAAUCCC | 301 | GGGAUUCAGCGCCGACGGG | 947 |
| ACCAGCACCAUGCAACUUU | 302 | ACCAGCACCAUGCAACUUU | 302 | AAAGUUGCAUGGUGCUGGU | 948 |
| GCGGGGUUUUUCUUGUUGA | 303 | GCGGGGUUUUUCUUGUUGA | 303 | UCAACAAGAAAAACCCCGC | 949 |
| AGACCACCAAAUGCCCCUA | 304 | AGACCACCAAAUGCCCCUA | 304 | UAGGGGCAUUUGGUGGUCU | 950 |
| CGCCAACUUACAAGGCCUU | 305 | CGCCAACUUACAAGGCCUU | 305 | AAGGCCUUGUAAGUUGGCG | 951 |
| GACCACCAAAUGCCCCUAU | 306 | GACCACCAAAUGCCCCUAU | 306 | AUAGGGGCAUUUGGUGGUC | 952 |
| GGCGGGGUUUUUCUUGUUG | 307 | GGCGGGGUUUUUCUUGUUG | 307 | CAACAAGAAAAACCCCGCC | 953 |
| AGGCGGGGUUUUUCUUGUU | 308 | AGGCGGGGUUUUUCUUGUU | 308 | AACAAGAAAAACCCCGCCU | 954 |
| UCGGCGCUGAAUCCCGCGG | 309 | UCGGCGCUGAAUCCCGCGG | 309 | CCGCGGGAUUCAGCGCCGA | 955 |
| ACCACCAAAUGCCCCUAUC | 310 | ACCACCAAAUGCCCCUAUC | 310 | GAUAGGGGCAUUUGGUGGU | 956 |
| CGGGGUUUUUCUUGUUGAC | 311 | CGGGGUUUUUCUUGUUGAC | 311 | GUCAACAAGAAAAACCCCG | 957 |
| ACCAUGCAACUUUUUCACC | 312 | ACCAUGCAACUUUUUCACC | 312 | GGUGAAAAAGUUGCAUGGU | 958 |
| CUGUAGGCAUAAAUUGGUC | 313 | CUGUAGGCAUAAAUUGGUC | 313 | GACCAAUUUAUGCCUACAG | 959 |
| GUGUGGAUUCGCACUCCUC | 314 | GUGUGGAUUCGCACUCCUC | 314 | GAGGAGUGCGAAUCCACAC | 960 |
| UGUAGGCAUAAAUUGGUCU | 315 | UGUAGGCAUAAAUUGGUCU | 315 | AGACCAAUUUAUGCCUACA | 961 |
| CACCAUGCAACUUUUUCAC | 316 | CACCAUGCAACUUUUUCAC | 316 | GUGAAAAAGUUGCAUGGUG | 962 |
| GUCGGCGCUGAAUCCCGCG | 317 | GUCGGCGCUGAAUCCCGCG | 317 | CGCGGGAUUCAGCGCCGAC | 963 |
| AUACUGCGGAACUCCUAGC | 318 | AUACUGCGGAACUCCUAGC | 318 | GCUAGGAGUUCCGCAGUAU | 964 |
| UACCAAUUUUCUUUUGUCU | 319 | UACCAAUUUUCUUUUGUCU | 319 | AGACAAAAGAAAAUUGGUA | 965 |
| AUGCCCCUAUCUUAUGAAC | 320 | AUGCCCCUAUCUUAUCAAC | 320 | GUUGAUAAGAUAGGGGCAU | 966 |
| CCAUACUGCGGAAGUCCUA | 321 | CCAUACUGCGGAACUCCUA | 321 | UAGGAGUUCCGCAGUAUGG | 967 |
| GUAGGCAUAAAUUGGUCUG | 322 | GUAGGCAUAAAUUGGUCUG | 322 | CAGACCAAUUUAUGCCUAC | 968 |
| CAUACUGCGGAACUCCUAG | 323 | CAUACUGCGGAACUCCUAG | 323 | CUAGGAGUUCCGCAGUAUG | 969 |
| AAAUGCCCCUAUCUUAUCA | 324 | AAAUGCCCCUAUCUUAUCA | 324 | UGAUAAGAUAGGGGCAUUU | 970 |
| AAUGCCCCUAUCUUAUCAA | 325 | AAUGCCCCUAUCUUAUCAA | 325 | UUGAUAAGAUAGGGGCAUU | 971 |
| CACCAGCACCAUGCAACUU | 326 | CACCAGCACCAUGCAACUU | 326 | AAGUUGCAUGGUGCUGGUG | 972 |
| UGAACCUUUACCCCGUUGC | 327 | UGAACCUUUACCCCGUUGC | 327 | GCAACGGGGUAAAGGUUCA | 973 |
| UGGAGACCACCGUGAACGC | 328 | UGGAGACCACCGUGAACGC | 328 | GCGUUCACGGUGGUCUCCA | 974 |
| GCCAACUUACAAGGCCUUU | 329 | GCCAACUUACAAGGCCUUU | 329 | AAAGGCCUUGUAAGUUGGC | 975 |
| UUACCAAUUUUCUUUUGUC | 330 | UUACCAAUUUUCUUUUGUC | 330 | GACAAAAGAAAAUUGGUAA | 976 |
| UCCUGCUGGUGGCUCCAGU | 331 | UCCUGCUGGUGGCUCCAGU | 331 | ACUGGAGCCACCAGGAGGA | 977 |
| UGUGCCUUCUCAUCUGCCG | 332 | UGUGCCUUCUCAUCUGCCG | 332 | CGGCAGAUGAGAAGGCACA | 978 |
| CCCCGUCUGUGCCUUCUCA | 333 | CCCCGUCUGUGCCUUCUCA | 333 | UGAGAAGGCACAGACGGGG | 979 |
| UUCGUAGGGCUUUCCCCCA | 334 | UUCGUAGGGCUUUCCCCCA | 334 | UGGGGGAAAGCCCUACGAA | 980 |
| CAGAAGAUCUCAAUCUCGG | 335 | CAGAAGAUCUCAAUCUCGG | 335 | CCGAGAUUGAGAUCUUCUG | 981 |
| UGCCCCUAUCUUAUCAACA | 336 | UGCCCCUAUCUUAUCAACA | 336 | UGUUGAUAAGAUAGGGGCA | 982 |
| GCAGAAGAUCUCAAUCUCG | 337 | GCAGAAGAUCUCAAUCUCG | 337 | CGAGAUUGAGAUCUUCUGC | 983 |
| AGCACCAUGCAACUUUUUC | 338 | AGCACCAUGCAACUUUUUC | 338 | GAAAAAGUUGCAUGGUGCU | 984 |
| CGUCUGUGCCUUCUCAUCU | 339 | CGUCUGUGCCUUCUCAUCU | 339 | AGAUGAGAAGGCACAGACG | 985 |
| CAGUGGUUCGUAGGGCUUU | 340 | CAGUGGUUCGUAGGGCUUU | 340 | AAAGCCCUACGAACCACUG | 986 |
| UGGUUCGUAGGGCUUUCCC | 341 | UGGUUCGUAGGGCUUUCCC | 341 | GGGAAAGCCCUACGAACCA | 987 |
| GCCCCUAUCUUAUCAACAC | 342 | GCCCCUAUCUUAUCAACAC | 342 | GUGUUGAUAAGAUAGGGGC | 988 |
| CCCGUCUGUGCCUUCUCAU | 343 | CCCGUCUGUGCCUUCUCAU | 343 | AUGAGAAGGCACAGACGGG | 989 |
| GCACCAUGCAACUUUUUCA | 344 | GCACCAUGCAACUUUUUCA | 344 | UGAAAAAGUUGCAUGGUGC | 990 |
| AGUGGUUCGUAGGGCUUUC | 345 | AGUGGUUCGUAGGGCUUUC | 345 | GAAAGCCCUACGAACCACU | 991 |
| GGUUCGUAGGGCUUUCCCC | 346 | GGUUCGUAGGGCUUUCCCC | 346 | GGGGAAAGCCCUACGAACC | 992 |
| GUGGUUCGUAGGGCUUUCC | 347 | GUGGUUCGUAGGGCUUUCC | 347 | GGAAAGCCCUACGAACCAC | 993 |
| AGAAGAUCUCAAUCUCGGG | 348 | AGAAGAUCUCAAUCUCGGG | 348 | CCCGAGAUUGAGAUCUUCU | 994 |
| CCGUCUGUGCCUUCUCAUC | 349 | CCGUCUGUGCCUUCUCAUC | 349 | GAUGAGAAGGCACAGACGG | 995 |
| UGGGGUGGAGCCCUCAGGC | 350 | UGGGGUGGAGCCCUCAGGC | 350 | GCCUGAGGGCUCCACCCCA | 996 |
| GACCACGGGGCGCACCUCU | 351 | GACCACGGGGCGCACCUCU | 351 | AGAGGUGCGCCCGGUGGUC | 997 |
| UUGUUCAGUGGUUCGUAGG | 352 | UUGUUCAGUGGUUCGUAGG | 352 | CCUACGAACCACUGAACAA | 998 |
| UGUUCAGUGGUUCGUAGGG | 353 | UGUUCAGUGGUUCGUAGGG | 353 | CCCUACGAACCACUGAACA | 999 |
| GUUCGUAGGGCUUUCCCCC | 354 | GUUCGUAGGGCUUUCCCCC | 354 | GGGGGAAAGCCCUACGAAC | 1000 |
| GGAGACCACCGUGAACGCC | 355 | GGAGACCACCGUGAACGCC | 355 | GGCGUUCACGGUGGUCUCC | 1001 |
| UUUGUUCAGUGGUUCGUAG | 356 | UUUGUUCAGUGGUUCGUAG | 356 | CUACGAACCACUGAACAAA | 1002 |
| CCGACCACGGGGCGCACCU | 357 | CCGACCACGGGGCGCACCU | 357 | AGGUGCGCCCCGUGGUCGG | 1003 |
| UCCCUCGCCUCGCAGACGA | 358 | UCCCUCGCCUCGCAGACGA | 358 | UCGUCUGCGAGGCGAGGGA | 1004 |
| GUGCCUUCUCAUCUGCCGG | 359 | GUGCCUUCUCAUCUGCCGG | 359 | CCGGCAGAUGAGAAGGCAC | 1005 |
| UUCAGUGGUUCGUAGGGCU | 360 | UUCAGUGGUUCGUAGGGCU | 360 | AGCCCUACGAACCACUGAA | 1006 |
| CGACCACGGGGCGCACCUC | 361 | CGACCACGGGGCGCACCUC | 361 | GAGGUGCGCCCCGUGGUCG | 1007 |
| GUUCAGUGGUUCGUAGGGC | 362 | GUUCAGUGGUUCGUAGGGC | 362 | GCCCUACGAACCACUGAAC | 1008 |
| UCAGUGGUUCGUAGGGCUU | 363 | UCAGUGGUUCGUAGGGCUU | 363 | AAGCCCUACGAACCACUGA | 1009 |
| UUCCUGCUGGUGGCUCCAG | 364 | UUCCUGCUGGUGGCUCCAG | 364 | CUGGAGCCACCAGCAGGAA | 1010 |
| CCCUCGCCUCGCAGACGAA | 365 | CCCUCGCCUCGCAGACGAA | 365 | UUCGUCUGCGAGGCGAGGG | 1011 |
| CCUCGCCUCGCAGACGAAG | 366 | CCUCGCCUCGCAGACGAAG | 366 | CUUCGUCUGCGAGGCGAGG | 1012 |
| CUGUGCCUUCUCAUCUGCC | 367 | CUGUGCCUUCUCAUCUGCC | 367 | GGCAGAUGAGAAGGCACAG | 1013 |
| ACCUCUGCCUAAUCAUCUC | 368 | ACCUCUGCCUAAUCAUCUC | 368 | GAGAUGAUUAGGCAGAGGU | 1014 |
| UCGUAGGGCUUUCCGCCAC | 369 | UCGUAGGGCUUUCCCCCAC | 369 | GUGGGGGAAAGCCCUACGA | 1015 |
| ACUUCCGGAAACUACUGUU | 370 | ACUUCCGGAAACUACUGUU | 370 | AACAGUAGUUUCCGGAAGU | 1016 |
| UCUGUGCCUUCUCAUCUGC | 371 | UCUGUGCCUUCUCAUCUGC | 371 | GCAGAUGAGAAGGCACAGA | 1017 |
| GUCUGUGCCUUCUCAUCUG | 372 | GUCUGUGCCUUCUCAUCUG | 372 | CAGAUGAGAAGGCACAGAC | 1018 |
| ACCUCUGCACGUCGCAUGG | 373 | ACCUCUGCACGUCGCAUGG | 373 | CCAUGCGACGUGCAGAGGU | 1019 |
| CAACGACCGACCUUGAGGC | 374 | CAACGACCGACCUUGAGGC | 374 | GCCUCAAGGUCGGUCGUUG | 1020 |
| UCAACGACCGACCUUGAGG | 375 | UCAACGACCGACCUUGAGG | 375 | CCUCAAGGUCGGUCGUUGA | 1021 |
| UCACCUCUGCACGUCGCAU | 376 | UCACCUCUGCACGUCGCAU | 376 | AUGCGACGUGCAGAGGUGA | 1022 |
| GUCAACGACCGACCUUGAG | 377 | GUCAACGACCGACCUUGAG | 377 | CUCAAGGUCGGUCGUUGAC | 1023 |
| CAAAUGCCCCUAUCUUAUC | 378 | CAAAUGCCCCUAUCUUAUC | 378 | GAUAAGAUAGGGGCAUUUG | 1024 |
| CACCUCUGCACGUCGCAUG | 379 | CACCUCUGCACGUCGCAUG | 379 | CAUGCGACGUGCAGAGGUG | 1025 |
| CACUUCCGGAAACUACUGU | 380 | CACUUCCGGAAACUACUGU | 380 | ACAGUAGUUUCCGGAAGUG | 1026 |
| ACACUUCCGGAAACUACUG | 381 | ACACUUCCGGAAACUACUG | 381 | CAGUAGUUUCCGGAAGUGU | 1027 |
| UGUCAACGACCGACCUUGA | 382 | UGUCAACGACCGACCUUGA | 382 | UCAAGGUCGGUCGUUGACA | 1028 |
| AUGUCAACGACCGACCUUG | 383 | AUGUCAACGACCGACCUUG | 383 | CAAGGUCGGUCGUUGACAU | 1029 |
| GCGCAUGCGUGGAACCUUU | 384 | GCGCAUGCGUGGAACCUUU | 384 | AAAGGUUCCACGCAUGCGC | 1030 |
| UCUUAUCAACACUUCCGGA | 385 | UCUUAUCAACACUUCCGGA | 385 | UCCGGAAGUGUUGAUAAGA | 1031 |
| AUUUGUUCAGUGGUUCGUA | 386 | AUUUGUUCAGUGGUUCGUA | 386 | UACGAACCACUGAACAAAU | 1032 |
| CCCUAUCUUAUCAACACUU | 387 | CCCUAUCUUAUCAACACUU | 387 | AAGUGUUGAUAAGAUAGGG | 1033 |
| UAUCUUAUCAACACUUCCG | 388 | UAUCUUAUCAACACUUCCG | 388 | CGGAAGUGUUGAUAAGAUA | 1034 |
| CCUCUGCACGUCGCAUGGA | 389 | CCUCUGCACGUCGCAUGGA | 389 | UCCAUGCGACGUGCAGAGG | 1035 |
| UGUGGAUUCGCACUCCUCC | 390 | UGUGGAUUCGCACUCCUCC | 390 | GGAGGAGUGCGAAUCCACA | 1036 |
| CCCCUAUCUUAUCAACACU | 391 | CCCCUAUCUUAUCAACACU | 391 | AGUGUUGAUAAGAUAGGGG | 1037 |
| CCUAUCUUAUCAACACUUC | 392 | CCUAUCUUAUCAACACUUC | 392 | GAAGUGUUGAUAAGAUAGG | 1038 |
| UUCACCUCUGCACGUCGCA | 393 | UUCACCUCUGCACGUCGCA | 393 | UGCGACGUGCAGAGGUGAA | 1039 |
| CUAUCUUAUCAACACUUCC | 394 | CUAUCUUAUCAACACUUCC | 394 | GGAAGUGUUGAUAAGAUAG | 1040 |
| AUCUUAUCAACACUUCCGG | 395 | AUCUUAUCAACACUUCCGG | 395 | CCGGAAGUGUUGAUAAGAU | 1041 |
| CAUUUGUUCAGUGGUUCGU | 396 | CAUUUGUUCAGUGGUUCGU | 396 | ACGAACCACUGAACAAAUG | 1042 |
| GGAAACUACUGUUGUUAGA | 397 | GG~AACUACUGUUGUUAGA | 397 | UCUAACAACAGUAGUUUCC | 1043 |
| UCCGGAAACUACUGUUGUU | 398 | UCCGGAAACUACUGUUGUU | 398 | AACAACAGUAGUUUCCGGA | 1044 |
| CCAACUUACAAGGCCUUUC | 399 | CCAACUUACAAGGCCUUUC | 399 | GAAAGGCCUUGUAAGUUGG | 1045 |
| CGGAAACUACUGUUGUUAG | 400 | CGGAAACUACUGUUGUUAG | 400 | CUAACAACAGUAGUUUCCG | 1046 |
| GAGACCACCGUGAACGCCC | 401 | GAGACCACCGUGAACGCCC | 401 | GGGCGUUCACGGUGGUCUC | 1047 |
| CUUCACCUCUGCACGUCGC | 402 | CUUCACCUCUGCACGUCGC | 402 | GCGACGUGCAGAGGUGAAG | 1048 |
| CCGGAAACUACUGUUGUUA | 403 | CCGGAAACUACUGUUGUUA | 403 | UAACAACAGUAGUUUCCGG | 1049 |
| CAACUUACAAGGCCUUUCU | 404 | CAACUUACAAGGCCUUUCU | 404 | AGAAAGGCCUUGUAAGUUG | 1050 |
| CGCUUCACCUCUGCACGUC | 405 | CGCUUCACCUCUGCACGUC | 405 | GACGUGCAGAGGUGAAGCG | 1051 |
| CAUAAGAGGACUCUUGGAC | 406 | CAUAAGAGGACUCUUGGAC | 406 | GUCCAAGAGUCCUCUUAUG | 1052 |
| GCUUCACCUCUGCACGUCG | 407 | GCUUCACCUCUGCACGUCG | 407 | CGACGUGCAGAGGUGAAGC | 1053 |
| AAGAUCUCAAUCUCGGGAA | 408 | AAGAUCUCAAUCUCGGGAA | 408 | UUCCCGAGAUUGAGAUCUU | 1054 |
| UUGGAGUGUGGAUUCGCAC | 409 | UUGGAGUGUGGAUUCGCAC | 409 | GUGCGAAUCCACACUCCAA | 1055 |
| UUUGGAGUGUGGAUUCGCA | 410 | UUUGGAGUGUGGAUUCGCA | 410 | UGCGAAUCCACACUCCAAA | 1056 |
| UUCCGGAAACUACUGUUGU | 411 | UUCCGGAAACUACUGUUGU | 411 | ACAACAGUAGUUUCCGGAA | 1057 |
| GAAACUACUGUUGUUAGAC | 412 | GAAACUACUGUUGUUAGAC | 412 | GUCUAACAACAGUAGUUUC | 1058 |
| GAAGAUCUCAAUCUCGGGA | 413 | GAAGAUCUCAAUCUCGGGA | 413 | UCCCGAGAUUGAGAUCUUC | 1059 |
| UGGGGGCCAAGUCUGUACA | 414 | UGGGGGCCAAGUCUGUACA | 414 | UGUACAGACUUGGCCCCCA | 1060 |
| CUUCCGGAAACUACUGUUG | 415 | CUUCCGGAAACUACUGUUG | 415 | CAACAGUAGUUUCCGGAAG | 1061 |
| CCAAAUGCCCCUAUCUUAU | 416 | CCAAAUGCCCCUAUCUUAU | 416 | AUAAGAUAGGGGCAUUUGG | 1062 |
| AACUACUGUUGUUAGACGA | 417 | AACUACUGUUGUUAGACGA | 417 | UCGUCUAACAACAGUAGUU | 1063 |
| GUCCUACUGUUCAAGCCUC | 418 | GUCCUACUGUUCAAGCCUC | 418 | GAGGCUUGAACAGUAGGAC | 1064 |
| CCUACUGUUCAAGCCUCCA | 419 | CCUACUGUUCAAGCCUCCA | 419 | UGGAGGCUUGAACAGUAGG | 1065 |
| AAUGUCAACGACCGACCUU | 420 | AAUGUCAACGACCGACCUU | 420 | AAGGUCGGUCGUUGACAUU | 1066 |
| UCCUACUGUUCAAGCCUCC | 421 | UCCUACUGUUCAAGCCUCC | 421 | GGAGGCUUGAACAGUAGGA | 1067 |
| AAACUACUGUUGUUAGACG | 422 | AAACUACUGUUGUUAGACG | 422 | CGUCUAACAACAGUAGUUU | 1068 |
| CUACUGUUCAAGCCUCCAA | 423 | CUACUGUUCAAGCCUCCAA | 423 | UUGGAGGCUUGAACAGUAG | 1069 |
| UGUCCUACUGUUCAAGCCU | 424 | UGUCCUACUGUUCAAGCCU | 424 | AGGCUUGAACAGUAGGACA | 1070 |
| UACUGUUCAAGCCUCCAAG | 425 | UACUGUUCAAGCCUCCAAG | 425 | CUUGGAGGCUUGAACAGUA | 1071 |
| GUGGGCCUCAGUCCGUUUC | 426 | GUGGGCCUCAGUCCGUUUC | 426 | GAAACGGACUGAGGCCCAC | 1072 |
| CUCCUGCCUCCACCAAUCG | 427 | CUCCUGCCUCCACCAAUCG | 427 | CGAUUGGUGGAGGCAGGAG | 1073 |
| UGGGCCUCAGUCCGUUUCU | 428 | UGGGCCUCAGUCCGUUUCU | 428 | AGAAACGGACUGAGGCCCA | 1074 |
| UGGGAGUGGGCCUCAGUCC | 429 | UGGGAGUGGGCCUCAGUCC | 429 | GGACUGAGGCCCACUCCCA | 1075 |
| CCUCCUGCCUCCACCAAUC | 430 | CCUCCUGCCUCCACCAAUC | 430 | GAUUGGUGGAGGCAGGAGG | 1076 |
| UAUGGGAGUGGGCCUCAGU | 431 | UAUGGGAGUGGGCCUCAGU | 431 | ACUGAGGCCCACUCCCAUA | 1077 |
| GCAUGCGUGGAACCUUUGU | 432 | GCAUGCGUGGAACCUUUGU | 432 | ACAAAGGUUCCACGCAUGC | 1078 |
| AUAAGGUGGGAAACUUUAC | 433 | AUAAGGUGGGAAACUUUAC | 433 | GUAAAGUUUCCCACCUUAU | 1079 |
| CGCAUGCGUGGAACCUUUG | 434 | CGCAUGCGUGGAACCUUUG | 434 | CAAAGGUUCCACGCAUGCG | 1080 |
| AUGUCCUACUGUUCAAGCC | 435 | AUGUCCUACUGUUCAAGCC | 435 | GGCUUGAACAGUAGGACAU | 1081 |
| UUUUUGCCUUCUGACUUCU | 436 | UUUUUGCCUUCUGACUUCU | 436 | AGAAGUCAGAAGGCAAAAA | 1082 |
| GGGCCUCAGUCCGUUUCUC | 437 | GGGCCUCAGUCCGUUUCUC | 437 | GAGAAACGGACUGAGGCCC | 1083 |
| CAUAAGGUGGGAAACUUUA | 438 | CAUAAGGUGGGAAACUUUA | 438 | UAAAGUUUCCCACCUUAUG | 1084 |
| GGAGUGGGCCUCAGUCCGU | 439 | GGAGUGGGCCUCAGUCCGU | 439 | ACGGACUGAGGCCCACUCC | 1085 |
| UGGAGUGUGGAUUCGCACU | 440 | UGGAGUGUGGAUUCGCACU | 440 | AGUGCGAAUCCACACUCCA | 1086 |
| AUGGGAGUGGGCCUCAGUC | 441 | AUGGGAGUGGGCCUCAGUC | 441 | GACUGAGGCCCACUCCCAU | 1087 |
| GAGUGGGCCUCAGUCCGUU | 442 | GAGUGGGCCUCAGUCCGUU | 442 | AACGGACUGAGGCCCACUC | 1088 |
| CAUGUCCUACUGUUCAAGC | 443 | CAUGUCCUACUGUUCAAGC | 443 | GCUUGAACAGUAGGACAUG | 1089 |
| GGGAGUGGGCCUCAGUCCG | 444 | GGGAGUGGGCCUCAGUCCG | 444 | CGGACUGAGGCCCACUCGC | 1090 |
| AGUGGGCCUCAGUCCGUUU | 445 | AGUGGGCCUCAGUCCGUUU | 445 | AAACGGACUGAGGCCCACU | 1091 |
| CCACCAAAUGCCCCUAUCU | 446 | CCACCAAAUGCCCCUAUCU | 446 | AGAUAGGGGCAUUUGGUGG | 1092 |
| UGUUCAUGUCCUACUGUUC | 447 | UGUUCAUGUCCUACUGUUC | 447 | GAACAGUAGGACAUGAACA | 1093 |
| ACCAAAUGCCCCUAUCUUA | 448 | ACCAAAUGCCCCUAUCUUA | 448 | UAAGAUAGGGGCAUUUGGU | 1094 |
| CACCAAAUGCCCCUAUCUU | 449 | CACCAAAUGCCCCUAUCUU | 449 | AAGAUAGGGGCAUUUGGUG | 1095 |
| UUGGGGGCCAAGUCUGUAC | 450 | UUGGGGGCCAAGUCUGUAC | 450 | GUACAGACUUGGCCCCCAA | 1096 |
| GUUCAUGUCCUACUGUUCA | 451 | GUUCAUGUCCUACUGUUCA | 451 | UGAACAGUAGGACAUGAAC | 1097 |
| UCAUGUCCUACUGUUCAAG | 452 | UCAUGUCCUACUGUUCAAG | 452 | CUUGAACAGUAGGACAUGA | 1098 |
| UUCUCGCCAACUUACAAGG | 453 | UUCUCGCCAACUUACAAGG | 453 | CCUUGUAAGUUGGCGAGAA | 1099 |
| UUUUGCCUUCUGACUUCUU | 454 | UUUUGCCUUCUGACUUCUU | 454 | AAGAAGUCAGAAGGCAAAA | 1100 |
| UCCUCAGGCCAUGCAGUGG | 455 | UCCUCAGGCCAUGCAGUGG | 455 | CCACUGCAUGGCCUGAGGA | 1101 |
| CAUGCGUGGAACCUUUGUG | 456 | CAUGCGUGGAACCUUUGUG | 456 | CACAAAGGUUCCACGCAUG | 1102 |
| UUCAUGUCCUACUGUUCAA | 457 | UUCAUGUCCUACUGUUCAA | 457 | UUGAACAGUAGGACAUGAA | 1103 |
| UGGACUCAUAAGGUGGGAA | 458 | UGGACUCAUAAGGUGGGAA | 458 | UUCCCACCUUAUGAGUCCA | 1104 |
| UUUCUCGCCAACUUACAAG | 459 | UUUCUCGCCAACUUACAAG | 459 | CUUGUAAGUUGGCGAGAAA | 1105 |
| UGCGCGGGACGUCCUUUGU | 460 | UGCGCGGGACGUCCUUUGU | 460 | ACAAAGGACGUCCCGCGCA | 1106 |
| CCGGACCGUGUGCACUUCG | 461 | CCGGACCGUGUGCACUUCG | 461 | CGAAGUGCACACGGUCCGG | 1107 |
| CAUCCUCAGGCCAUGCAGU | 462 | CAUCCUCAGGCCAUGCAGU | 462 | ACUGCAUGGCCUGAGGAUG | 1108 |
| GGACUCAUAAGGUGGGAAA | 463 | GGACUCAUAAGGUGGGAAA | 463 | UUUCCCACCUUAUGAGUCC | 1109 |
| CUGCGCGGGACGUCCUUUG | 464 | CUGCGCGGGACGUCCUUUG | 464 | CAAAGGACGUCCCGCGCAG | 1110 |
| AUCCUCAGGCCAUGCAGUG | 465 | AUCCUCAGGCCAUGCAGUG | 465 | CACUGCAUGGCCUGAGGAU | 1111 |
| GACCGUGUGCACUUCGCUU | 466 | GACCGUGUGCACUUCGCUU | 466 | AAGCGAAGUGCACACGGUC | 1112 |
| ACUUUCUCGCCAACUUACA | 467 | ACUUUCUCGCCAACUUACA | 467 | UGUAAGUUGGCGAGAAAGU | 1113 |
| GGACCGUGUGCACUUCGCU | 468 | GGACCGUGUGCACUUCGCU | 468 | AGCGAAGUGCACACGGUCC | 1114 |
| CUUUCUCGCCAACUUACAA | 469 | CUUUCUCGCCAACUUACAA | 469 | UUGUAAGUUGGCGAGAAAG | 1115 |
| ACCGUGUGCACUUCGCUUC | 470 | ACCGUGUGCACUUCGCUUC | 470 | GAAGCGAAGUGCACACGGU | 1116 |
| UGCUGCCAACUGGAUCCUG | 471 | UGCUGCCAACUGGAUCCUG | 471 | CAGGAUCCAGUUGGCAGCA | 1117 |
| GCUGCCAACUGGAUCCUGC | 472 | GCUGCCAACUGGAUCCUGC | 472 | GCAGGAUCCAGUUGGCAGC | 1118 |
| CGGACCGUGUGCACUUCGC | 473 | CGGACCGUGUGCACUUCGC | 473 | GCGAAGUGCACACGGUCCG | 1119 |
| GCUGGUGGCUCCAGUUCAG | 474 | GCUGGUGGCUCCAGUUCAG | 474 | CUGAACUGGAGCCACCAGC | 1120 |
| UGCCUUCUGACUUCUUUCC | 475 | UGCCUUCUGACUUCUUUCC | 475 | GGAAAGAAGUCAGAAGGCA | 1121 |
| UCUCGCCAACUUACAAGGC | 476 | UCUCGCCAACUUACAAGGC | 476 | GCCUUGUAAGUUGGCGAGA | 1122 |
| CUCUUCAUCCUGCUGCUAU | 477 | CUCUUCAUCCUGCUGCUAU | 477 | AUAGCAGCAGGAUGAAGAG | 1123 |
| UGCCAACUGGAUCCUGCGC | 478 | UGCCAACUGGAUCCUGCGC | 478 | GCGCAGGAUCCAGUUGGCA | 1124 |
| CUUCAUCCUGCUGCUAUGC | 479 | CUUCAUCCUGCUGCUAUGC | 479 | GCAUAGCAGCAGGAUGAAG | 1125 |
| CCAACUGGAUCCUGCGCGG | 480 | CCAACUGGAUCCUGCGCGG | 480 | CCGCGCAGGAUCCAGUUGG | 1126 |
| GGUGGAGCCCUCAGGCUCA | 481 | GGUGGAGCCCUCAGGCUCA | 481 | UGAGCCUGAGGGCUCCACC | 1127 |
| UGCUGGUGGCUCCAGUUCA | 482 | UGCUGGUGGCUCCAGUUCA | 482 | UGAACUGGAGCCACCAGCA | 1128 |
| UCAUCCUGCUGCUAUGCCU | 483 | UCAUCCUGCUGCUAUGCCU | 483 | AGGCAUAGCAGCAGGAUGA | 1129 |
| GGGUGGAGCCCUCAGGCUC | 484 | GGGUGGAGCCCUCAGGCUC | 484 | GAGCCUGAGGGCUCCACCC | 1130 |
| GGCCAUCAGCGCAUGCGUG | 485 | GGCCAUCAGCGCAUGCGUG | 485 | CACGCAUGCGCUGAUGGCC | 1131 |
| UUCAUCCUGCUGCUAUGCC | 486 | UUCAUCCUGCUGCUAUGCC | 486 | GGCAUAGCAGCAGGAUGAA | 1132 |
| UCUUCAUCCUGCUGCUAUG | 487 | UCUUCAUCCUGCUGCUAUG | 487 | CAUAGCAGCAGGAUGAAGA | 1133 |
| GCCAACUGGAUCCUGCGCG | 488 | GCCAACUGGAUCCUGCGCG | 488 | CGCGCAGGAUCCAGUUGGC | 1134 |
| CUGCCAACUGGAUCCUGCG | 489 | CUGCCAACUGGAUCCUGCG | 489 | CGCAGGAUCCAGUUGGCAG | 1135 |
| CUCGCCAACUUACAAGGCC | 490 | CUCGCCAACUUACAAGGCC | 490 | GGCCUUGUAAGUUGGCGAG | 1136 |
| CCUCUUCAUCCUGCUGCUA | 491 | CCUCUUCAUCCUGCUGCUA | 491 | UAGCAGCAGGAUGAAGAGG | 1137 |
| ACUGGAUCCUGCGCGGGAC | 492 | ACUGGAUCCUGCGCGGGAC | 492 | GUCCCGCGCAGGAUCCAGU | 1138 |
| GGGGUGGAGCCCUCAGGCU | 493 | GGGGUGGAGCCCUCAGGCU | 493 | AGCCUGAGGGCUCCACCCC | 1139 |
| AACUGGAUCCUGCGCGGGA | 494 | AACUGGAUCCUGCGCGGGA | 494 | UCCCGCGCAGGAUCCAGUU | 1140 |
| CUUGGACUCAUAAGGUGGG | 495 | CUUGGACUCAUAAGGUGGG | 495 | CCCACCUUAUGAGUCCAAG | 1141 |
| CUGCCGGACCGUGUGCACU | 496 | CUGCCGGACCGUGUGCACU | 496 | AGUGCACACGGUCCGGCAG | 1142 |
| CCUGCGCGGGACGUCCUUU | 497 | CCUGCGCGGGACGUCCUUU | 497 | AAAGGACGUCCCGCGCAGG | 1143 |
| GAUCCUGCGCGGGACGUCC | 498 | GAUCCUGCGCGGGACGUCC | 498 | GGACGUCCCGCGCAGGAUC | 1144 |
| CCUUGGACUCAUAAGGUGG | 499 | CCUUGGACUCAUAAGGUGG | 499 | CCACCUUAUGAGUCCAAGG | 1145 |
| UGCCGGACCGUGUGCACUU | 500 | UGCCGGACCGUGUGCACUU | 500 | AAGUGCACACGGUCCGGCA | 1146 |
| AUCCUGCGCGGGACGUCCU | 501 | AUCCUGCGCGGGACGUCCU | 501 | AGGACGUCCCGCGCAGGAU | 1147 |
| GCCAUCAGCGCAUGCGUGG | 502 | GCCAUCAGCGCAUGCGUGG | 502 | CCACGCAUGCGCUGAUGGC | 1148 |
| UUGCCUUCUGACUUCUUUC | 503 | UUGCCUUCUGACUUCUUUC | 503 | GAAAGAAGUCAGAAGGCAA | 1149 |
| CAACUGGAUCCUGCGCGGG | 504 | CAACUGGAUCCUGCGCGGG | 504 | CCCGCGCAGGAUCCAGUUG | 1150 |
| UGGAUCCUGCGCGGGACGU | 505 | UGGAUCCUGCGCGGGACGU | 505 | ACGUCCCGCGCAGGAUCCA | 1151 |
| UGCUCAAGGAACCUCUAUG | 506 | UGCUCAAGGAACCUCUAUG | 506 | CAUAGAGGUUCCUUGAGCA | 1152 |
| UCCUGCGCGGGACGUCCUU | 507 | UCCUGCGCGGGACGUCCUU | 507 | AAGGACGUCCCGCGCAGGA | 1153 |
| UUUGCCUUCUGACUUCUUU | 508 | UUUGCCUUCUGACUUCUUU | 508 | AAAGAAGUCAGAAGGCAAA | 1154 |
| GCCGGACCGUGUGCACUUC | 509 | GCCGGACCGUGUGCACUUC | 509 | GAAGUGCACACGGUCCGGC | 1155 |
| GGAUCCUGCGCGGGACGUC | 510 | GGAUCCUGCGCGGGACGUC | 510 | GACGUCCCGCGCAGGAUCC | 1156 |
| UCCUCUUCAUCCUGCUGCU | 511 | UCCUCUUCAUCCUGCUGCU | 511 | AGCAGCAGGAUGAAGAGGA | 1157 |
| CUGGAUCCUGCGCGGGACG | 512 | CUGGAUCCUGCGCGGGACG | 512 | CGUCCGGCGGAGGAUCCAG | 1158 |
| GCUCAAGGAACCUCUAUGU | 513 | GCUCAAGGAACCUCUAUGU | 513 | ACAUAGAGGUUCCUUGAGC | 1159 |
| UCAUCCUCAGGCCAUGCAG | 514 | UCAUCCUCAGGCCAUGCAG | 514 | CUGCAUGGCCUGAGGAUGA | 1160 |
| UUCCUCUUCAUCCUGCUGC | 515 | UUCCUCUUCAUCCUGCUGC | 515 | GCAGCAGGAUGAAGAGGAA | 1161 |
| UCCUUUGUUUACGUCCCGU | 516 | UCCUUUGUUUACGUCCCGU | 516 | ACGGGACGUAAACAAAGGA | 1162 |
| GAGCCCUCAGGCUCAGGGC | 517 | GAGCCCUCAGGCUCAGGGC | 517 | GCCCUGAGCCUGAGGGCUC | 1163 |
| CCUUUGUUUACGUCCCGUC | 518 | CCUUUGUUUACGUCCCGUC | 518 | GACGGGACGUAAACAAAGG | 1164 |
| UUGGGGUGGAGCCCUCAGG | 519 | UUGGGGUGGAGCCCUCAGG | 519 | CCUGAGGGCUCCACCCCAA | 1165 |
| AUCAACACUUCCGGAAACU | 520 | AUCAACACUUCCGGAAACU | 520 | AGUUUCCGGAAGUGUUGAU | 1166 |
| ACGUCCUUUGUUUACGUCC | 521 | ACGUCCUUUGUUUACGUCC | 521 | GGACGUAAACAAAGGACGU | 1167 |
| GGACGUCCUUUGUUUACGU | 522 | GGACGUCCUUUGUUUACGU | 522 | ACGUAAACAAAGGACGUCC | 1168 |
| GGAGCCCUCAGGCUCAGGG | 523 | GGAGCCCUCAGGCUCAGGG | 523 | CCCUGAGCCUGAGGGCUCC | 1169 |
| GUCCUUUGUUUACGUCCCG | 524 | GUCCUUUGUUUACGUCCCG | 524 | CGGGACGUAAACAPAGGAC | 1170 |
| AUGAUGUGGUAUUGGGGGC | 525 | AUGAUGUGGUAUUGGGGGC | 525 | GCCCCCAAUACCACAUCAU | 1171 |
| UCUGCCGGACGGUGUGGAC | 526 | UCUGCCGGACCGUGUGCAC | 526 | GUGCACACGGUCCGGCAGA | 1172 |
| UAUCAACACUUCCGGAAAC | 527 | UAUCAACACUUCCGGAAAC | 527 | GUUUCCGGAAGUGUUGAUA | 1173 |
| CGUCCUUUGUUUACGUCCC | 528 | CGUCCUUUGUUUACGUCCC | 528 | GGGACGUPAACAAAGGACG | 1174 |
| GAUGAUGUGGUAUUGGGGG | 529 | GAUGAUGUGGUAUUGGGGG | 529 | CCCCCAAUACCACAUCAUC | 1175 |
| GACGUCCUUUGUUUACGUC | 530 | GACGUCCUUUGUUUACGUC | 530 | GACGUAAACAAAGGACGUC | 1176 |
| GGAUGAUGUGGUAUUGGGG | 531 | GGAUGAUGUGGUAUUGGGG | 531 | CCCCAAUACCACAUCAUCC | 1177 |
| UGGAUGAUGUGGUAUUGGG | 532 | UGGAUGAUGUGGUAUUGGG | 532 | CCCAAUACCACAUCAUCCA | 1178 |
| AUGGAUGAUGUGGUAUUGG | 533 | AUGGAUGAUGUGGUAUUGG | 533 | CCAAUACCACAUCAUCCAU | 1179 |
| GGGACGUCCUUUGUUUACG | 534 | GGGACGUCCUUUGUUUACG | 534 | CGUAAACAAAGGACGUCCC | 1180 |
| AUCAAGGUAUGUUGCCCGU | 535 | AUCAAGGUAUGUUGCCCGU | 535 | ACGGGCAACAUACCUUGAU | 1181 |
| ACCUGUAUUCCCAUCCCAU | 536 | ACCUGUAUUCCCAUCCCAU | 536 | AUGGGAUGGGAAUACAGGU | 1182 |
| UAUCAAGGUAUGUUGCCCG | 537 | UAUCAAGGUAUGUUGCCCG | 537 | CGGGCAACAUACCUUGAUA | 1183 |
| CACCUGUAUUCCCAUCCCA | 538 | CACCUGUAUUCCCAUCCCA | 538 | UGGGAUGGGAAUACAGGUG | 1184 |
| UGCACCUGUAUUCCCAUCC | 539 | UGCACCUGUAUUCCCAUCC | 539 | GGAUGGGAAUACAGGUGCA | 1185 |
| UAUAUGGAUGAUGUGGUAU | 540 | UAUAUGGAUGAUGUGGUAU | 540 | AUACCACAUCAUCCAUAUA | 1186 |
| UAUGGAUGAUGUGGUAUUG | 541 | UAUGGAUGAUGUGGUAUUG | 541 | CAAUACCACAUCAUCCAUA | 1187 |
| UUGGACUCAUAAGGUGGGA | 542 | UUGGACUCAUAAGGUGGGA | 542 | UCCCACCUUAUGAGUCCAA | 1188 |
| UGGAGCCCUCAGGCUCAGG | 543 | UGGAGCCCUCAGGCUCAGG | 543 | CCUGAGCCUGAGGGCUCCA | 1189 |
| CCUGUAUUCCCAUCCCAUC | 544 | CCUGUAUUCCCAUCCCAUC | 544 | GAUGGGAUGGGAAUACAGG | 1190 |
| CGGGACGUCCUUUGUUUAC | 545 | CGGGACGUCCUUUGUUUAC | 545 | GUAAACAAAGGACGUCCCG | 1191 |
| AUAUGGAUGAUGUGGUAUU | 546 | AUAUGGAUGAUGUGGUAUU | 546 | AAUACCACAUCAUCCAUAU | 1192 |
| GCACCUGUAUUCCCAUCCC | 547 | GCACCUGUAUUCCCAUCCC | 547 | GGGAUGGGAAUACAGGUGC | 1193 |
| GUGGAGCCCUCAGGCUCAG | 548 | GUGGAGCCCUCAGGCUCAG | 548 | CUGAGCCUGAGGGCUCCAC | 1194 |
| CGCGGGACGUCCUUUGUUU | 549 | CGCGGGACGUCCUUUGUUU | 549 | AAACAAAGGACGUCCCGCG | 1195 |
| GCUCCUCUGCCGAUCCAUA | 550 | GCUCCUCUGCCGAUCCAUA | 550 | UAUGGAUCGGCAGAGGAGC | 1196 |
| UGAUGUGGUAUUGGGGGCC | 551 | UGAUGUGGUAUUGGGGGCC | 551 | GGCCCCCAAUACCACAUCA | 1197 |
| CAGCGCAUGCGUGGAACCU | 552 | CAGCGCAUGCGUGGAACCU | 552 | AGGUUCCACGCAUGCGCUG | 1198 |
| AGCGCAUGCGUGGAACCUU | 553 | AGCGCAUGCGUGGAACCUU | 553 | AAGGUUCCACGCAUGCGCU | 1199 |
| AUGUGGUAUUGGGGGCCAA | 554 | AUGUGGUAUUGGGGGCCAA | 554 | UUGGCCCCCAAUACCACAU | 1200 |
| UUUCCUGCUGGUGGCUCCA | 555 | UUUCCUGCUGGUGGCUCCA | 555 | UGGAGCCACCAGCAGGAAA | 1201 |
| GAUCUCAAUCUCGGGAAUC | 556 | GAUCUCAAUCUCGGGAAUC | 556 | GAUUCCCGAGAUUGAGAUC | 1202 |
| GCGGGACGUCCUUUGUUUA | 557 | GCGGGACGUCCUUUGUUUA | 557 | UAAACAAAGGACGUCCCGC | 1203 |
| GAUGUGGUAUUGGGGGCCA | 558 | GAUGUGGUAUUGGGGGCCA | 558 | UGGCCCCCAAUACCACAUC | 1204 |
| CUUAUCAACACUUCCGGAA | 559 | CUUAUCAACACUUCCGGAA | 559 | UUCCGGAAGUGUUGAUAAG | 1205 |
| GGCUCCUCUGCCGAUCCAU | 560 | GGCUCCUCUGCCGAUCCAU | 560 | AUGGAUCGGCAGAGGAGCC | 1206 |
| CAAUGUCAACGACCGACCU | 561 | CAAUGUCAACGACCGACCU | 561 | AGGUCGGUCGUUGACAUUG | 1207 |
| CUGGUGGCUCCAGUUCAGG | 562 | CUGGUGGCUCCAGUUCAGG | 562 | CCUGAACUGGAGCCACCAG | 1208 |
| UCCCCAACCUCCAAUCACU | 563 | UCCCCAACCUCCAAUCACU | 563 | AGUGAUUGGAGGUUGGGGA | 1209 |
| CCAUCAGCGCAUGCGUGGA | 564 | CCAUCAGCGCAUGCGUGGA | 564 | UCCACGCAUGCGCUGAUGG | 1210 |
| UUAUCAACACUUCCGGAAA | 565 | UUAUCAACACUUCCGGAAA | 565 | UUUCCGGAAGUGUUGAUAA | 1211 |
| UCAACACUUCCGGAAACUA | 566 | UCAACACUUCCGGAAACUA | 566 | UAGUUUCCGGAAGUGUUGA | 1212 |
| CAACACUUCCGGAAACUAC | 567 | CAACACUUCCGGAAACUAC | 567 | GUAGUUUCCGGAAGUGUUG | 1213 |
| GCAGUCCCCAACCUCCAAU | 568 | GCAGUCCCCAACCUCCAAU | 568 | AUUGGAGGUUGGGGACUGC | 1214 |
| AACACUUCCGGAAACUACU | 569 | AACACUUCCGGAAACUACU | 569 | AGUAGUUUCCGGAAGUGUU | 1215 |
| CAGUCCCCAACCUCCAAUC | 570 | CAGUCCCCAACCUCCAAUC | 570 | GAUUGGAGGUUGGGGACUG | 1216 |
| GUCCCCAACCUCCAAUCAC | 571 | GUCCCCAACCUCCAAUCAC | 571 | GUGAUUGGAGGUUGGGGAC | 1217 |
| AUUUUCUUUUGUCUUUGGG | 572 | AUUUUCUUUUGUCUUUGGG | 572 | CCCAAAGACAAAAGAAAAU | 1218 |
| AGUCCCCAACCUCCAAUCA | 573 | AGUCCCCAACCUCCAAUCA | 573 | UGAUUGGAGGUUGGGGACU | 1219 |
| GUGCCUUGGGUGGCUUUGG | 574 | GUGCCUUGGGUGGCUUUGG | 574 | CCAAAGCCACCCAAGGCAC | 1220 |
| CCACCAAUCGGCAGUCAGG | 575 | CCACCAAUCGGCAGUCAGG | 575 | CCUGACUGCCGAUUGGUGG | 1221 |
| UGCCUUGGGUGGCUUUGGG | 576 | UGCCUUGGGUGGCUUUGGG | 576 | CCCAAAGCCACCCAAGGCA | 1222 |
| UGUGCCUUGGGUGGCUUUG | 577 | UGUGCCUUGGGUGGCUUUG | 577 | CAAAGCCACCCAAGGCACA | 1223 |
| CCUGCCUCCACCAAUCGGC | 578 | CCUGCCUCCACCAAUCGGC | 578 | GCCGAUUGGUGGAGGCAGG | 1224 |
| GUUAUAUGGAUGAUGUGGU | 579 | GUUAUAUGGAUGAUGUGGU | 579 | ACCACAUCAUCCAUAUAAC | 1225 |
| AUCAGCGCAUGCGUGGAAC | 580 | AUCAGCGCAUGCGUGGAAC | 580 | GUUCCACGCAUGCGCUGAU | 1226 |
| UGGCUUUCAGUUAUAUGGA | 581 | UGGCUUUCAGUUAUAUGGA | 581 | UCCAUAUAACUGAAAGCCA | 1227 |
| GGCUUUCAGUUAUAUGGAU | 582 | GGCUUUCAGUUAUAUGGAU | 582 | AUCCAUAUAACUGAAAGCC | 1228 |
| AGAUCUCAAUCUCGGGAAU | 583 | AGAUCUCAAUCUCGGGAAU | 583 | AUUCCCGAGAUUGAGAUCU | 1229 |
| UCCUGCCUCCACCAAUCGG | 584 | UCCUGCCUCCACCAAUCGG | 584 | CCGAUUGGUGGAGGCAGGA | 1230 |
| UCAGCGCAUGCGUGGAACC | 585 | UCAGCGCAUGCGUGGAACC | 585 | GGUUCCACGCAUGCGCUGA | 1231 |
| CAUCAGCGCAUGCGUGGAA | 586 | CAUCAGCGCAUGCGUGGAA | 586 | UUCCACGCAUGCGCUGAUG | 1232 |
| CUGCCUCCACCAAUCGGCA | 587 | CUGCCUCCACCAAUCGGCA | 587 | UGCCGAUUGGUGGAGGGAG | 1233 |
| UCCACCAAUCGGCAGUCAG | 588 | UCCACCAAUCGGCAGUCAG | 588 | CUGACUGCCGAUUGGUGGA | 1234 |
| UCAAGGUAUGUUGCCCGUU | 589 | UCAAGGUAUGUUGCCCGUU | 589 | AACGGGCAACAUACCUUGA | 1235 |
| GCUUUCAGUUAUAUGGAUG | 590 | GCUUUCAGUUAUAUGGAUG | 590 | CAUCCAUAUAACUGAAAGC | 1236 |
| UGCCUCCACCAAUCGGCAG | 591 | UGCCUCCACCAAUCGGCAG | 591 | CUGCCGAUUGGUGGAGGCA | 1237 |
| GCCUCCACCAAUCGGCAGU | 592 | GCCUCCACCAAUCGGCAGU | 592 | ACUGCCGAUUGGUGGAGGC | 1238 |
| UUUCAGUUAUAUGGAUGAU | 593 | UUUCAGUUAUAUGGAUGAU | 593 | AUCAUCCAUAUAACUGAAA | 1239 |
| AGUUAUAUGGAUGAUGUGG | 594 | AGUUAUAUGGAUGAUGUGG | 594 | CCACAUCAUCCAUAUAACU | 1240 |
| CUUUCAGUUAUAUGGAUGA | 595 | CUUUCAGUUAUAUGGAUGA | 595 | UCAUCCAUAUAACUGAAAG | 1241 |
| UCUGCACGUCGCAUGGAGA | 596 | UCUGCACGUCGCAUGGAGA | 596 | UCUCCAUGCGACGUGCAGA | 1242 |
| UUCUUUUGUCUUUGGGUAU | 597 | UUCUUUUGUCUUUGGGUAU | 597 | AUACCCAAAGACAAAAGAA | 1243 |
| UUUCUUUUGUCUUUGGGUA | 598 | UUUCUUUUGUCUUUGGGUA | 598 | UACCCAAAGACAAAAGAAA | 1244 |
| CACGUCGCAUGGAGACCAC | 599 | CACGUCGCAUGGAGACCAC | 599 | GUGGUCUCCAUGCGACGUG | 1245 |
| CUUUGUUUACGUCCCGUCG | 600 | CUUUGUUUACGUCCCGUCG | 600 | CGACGGGACGUAAACAAAG | 1246 |
| UUUGUUUACGUCCCGUCGG | 601 | UUUGUUUACGUCCCGUCGG | 601 | CCGACGGGACGUAAACAAA | 1247 |
| UGCACGUCGCAUGGAGACC | 602 | UGCACGUCGCAUGGAGACC | 602 | GGUCUCCAUGCGACGUGCA | 1248 |
| GCACGUCGCAUGGAGACCA | 603 | GCACGUCGCAUGGAGACCA | 603 | UGGUCUCCAUGCGACGUGC | 1249 |
| CGCAUGGAGACCACCGUGA | 604 | CGCAUGGAGACCACGGUGA | 604 | UCACGGUGGUCUCCAUGCG | 1250 |
| UCGCAUGGAGACCACCGUG | 605 | UCGCAUGGAGACCACCGUG | 605 | CACGGUGGUCUCCAUGCGA | 1251 |
| UUUUCUUUUGUCUUUGGGU | 606 | UUUUCUUUUGUCUUUGGGU | 606 | ACCCAAAGACAAAAGAAAA | 1252 |
| GUCGCAUGGAGACCACCGU | 607 | GUCGCAUGGAGACCACCGU | 607 | ACGGUGGUCUCCAUGCGAC | 1253 |
| CUCUGCACGUCGCAUGGAG | 608 | CUCUGCACGUCGCAUGGAG | 608 | CUCCAUGCGACGUGCAGAG | 1254 |
| GCAAUGUCAACGACCGACC | 609 | GCAAUGUCAACGACCGACC | 609 | GGUCGGUCGUUGACAUUGC | 1255 |
| CUGCACGUCGCAUGGAGAC | 610 | CUGCACGUCGCAUGGAGAC | 610 | GUCUCCAUGCGACGUGCAG | 1256 |
| CGUCGCAUGGAGACCACCG | 611 | CGUCGCAUGGAGACCACCG | 611 | CGGUGGUCUCCAUGCGACG | 1257 |
| ACGUCGCAUGGAGACCACC | 612 | ACGUCGCAUGGAGACCACC | 612 | GGUGGUCUCCAUGCGACGU | 1258 |
| UUUGUCUUUGGGUAUACAU | 613 | UUUGUCUUUGGGUAUACAU | 613 | AUGUAUACCCAAAGACAAA | 1259 |
| UGUGGUUUCACAUUUCCUG | 614 | UGUGGUUUCACAUUUCCUG | 614 | CAGGAAAUGUGAAACCACA | 1260 |
| UCUUUUGUCUUUGGGUAUA | 615 | UCUUUUGUCUUUGGGUAUA | 615 | UAUACCCAAAGACAAAAGA | 1261 |
| CUUUUGUCUUUGGGUAUAC | 616 | CUUUUGUCUUUGGGUAUAC | 616 | GUAUACCCAAAGACAAAAG | 1262 |
| UUUUGUCUUUGGGUAUACA | 617 | UUUUGUCUUUGGGUAUACA | 617 | UGUAUACCCAAAGACAAAA | 1263 |
| CCUUCUCAUCUGCCGGACC | 618 | CCUUCUCAUCUGCCGGACC | 618 | GGUCCGGCAGAUGAGAAGG | 1264 |
| AUCUGCCGGACCGUGUGCA | 619 | AUCUGCCGGACCGUGUGCA | 619 | UGCACACGGUCCGGCAGAU | 1265 |
| CUCGCCUCGCAGACGAAGG | 620 | CUCGCCUCGCAGACGAAGG | 620 | CCUUCGUCUGCGAGGCGAG | 1266 |
| UCAUCUGCCGGACCGUGUG | 621 | UCAUCUGCCGGACGGUGUG | 621 | CACACGGUCCGGCAGAUGA | 1267 |
| UUGUUUACGUCCCGUCGGC | 622 | UUGUUUACGUCCCGUCGGC | 622 | GCCGACGGGACGUAAACAA | 1268 |
| UGUUUACGUCCCGUCGGCG | 623 | UGUUUACGUCCCGUCGGCG | 623 | CGCCGACGGGACGUAAACA | 1269 |
| CACCAAUCGGCAGUCAGGA | 624 | CACCAAUCGGCAGUCAGGA | 624 | UCCUGACUGCCGAUUGGUG | 1270 |
| UUUACGUCCCGUCGGCGCU | 625 | UUUACGUCCCGUCGGCGCU | 625 | AGCGCCGACGGGACGUAAA | 1271 |
| GUUUACGUCCCGUCGGCGC | 626 | GUUUACGUCCCGUCGGCGC | 626 | GCGCCGACGGGACGUAAAC | 1272 |
| CAUCUGCCGGACCGUGUGC | 627 | CAUCUGCCGGACGGUGUGC | 627 | GCACACGGUCCGGCAGAUG | 1273 |
| UCUUUUGGAGUGUGGAUUC | 628 | UCUUUUGGAGUGUGGAUUC | 628 | GAAUCCACACUCCAAAAGA | 1274 |
| UCGCCUCGCAGACGAAGGU | 629 | UCGCCUCGCAGACGAAGGU | 629 | ACCUUCGUCUGCGAGGCGA | 1275 |
| GCCUCGCAGACGAAGGUCU | 630 | GCCUCGCAGACGAAGGUCU | 630 | AGACCUUCGUCUGCGAGGC | 1276 |
| CUCAUCUGCCGGACCGUGU | 631 | CUCAUCUGCCGGACCGUGU | 631 | ACACGGUCCGGCAGAUGAG | 1277 |
| UGAGGCAUACUUCAAAGAC | 632 | UGAGGCAUACUUCAAAGAC | 632 | GUCUUUGAAGUAUGCCUCA | 1278 |
| UGGCUUUGGGGCAUGGACA | 633 | UGGCUUUGGGGCAUGGACA | 633 | UGUCCAUGCCCCAAAGCCA | 1279 |
| GGCUUUGGGGCAUGGACAU | 634 | GGCUUUGGGGCAUGGACAU | 634 | AUGUCCAUGCCCCAAAGCC | 1280 |
| CUUUUGGAGUGUGGAUUCG | 635 | CUUUUGGAGUGUGGAUUCG | 635 | CGAAUCCACACUCCAAAAG | 1281 |
| UCUCUUUUUUGCCUUCUGA | 636 | UCUCUUUUUUGCCUUCUGA | 636 | UCAGAAGGCAAAAAAGAGA | 1282 |
| ACCAAUUUUCUUUUGUCUU | 637 | ACCAAUUUUCUUUUGUCUU | 637 | AAGACAAAAGAAAAUUGGU | 1283 |
| CUUCUCAUCUGCCGGACCG | 638 | CUUCUCAUCUGCCGGACCG | 638 | CGGUCCGGCAGAUGAGAAG | 1284 |
| CCUCCUCCUGCCUCCACCA | 639 | CCUCCUCCUGCCUCCACCA | 639 | UGGUGGAGGCAGGAGGAGG | 1285 |
| UUUUGGAGUGUGGAUUCGC | 640 | UUUUGGAGUGUGGAUUCGC | 640 | GCGAAUCCACACUCCAAAA | 1286 |
| UUCUCAUCUGCCGGACCGU | 641 | UUCUCAUCUGCCGGACCGU | 641 | ACGGUCCGGCAGAUGAGAA | 1287 |
| AAUUUUCUUUUGUCUUUGG | 642 | AAUUUUCUUUUGUCUUUGG | 642 | CCAAAGACAAAAGAAAAUU | 1288 |
| CGCCUCGCAGACGAAGGUC | 643 | CGCCUCGCAGACGAAGGUC | 643 | GACCUUCGUCUGCGAGGCG | 1289 |
| CCAAUUUUCUUUUGUCUUU | 644 | CCAAUUUUCUUUUGUCUUU | 644 | AAAGACAAAAGAAAAUUGG | 1290 |
| CAAUUUUCUUUUGUCUUUG | 645 | CAAUUUUCUUUUGUCUUUG | 645 | CAAAGACAAAAGAAAAUUG | 1291 |
| UCUCAUCUGCCGGACCGUG | 646 | UCUCAUCUGCCGGACCGUG | 646 | CACGGUCCGGCAGAUGAGA | 1292 |

HBV Composite

The 3′-ends of the Upper sequence and the Lower sequence of the siRNA construct can include an overhang sequence, for example about 1, 2, 3, or 4 nucleotides in length, preferably 2 nucleotides in length, wherein the overhanging sequence of the lower sequence is optionally complementary to a portion of the target sequence. The upper sequence is also referred to as the sense strand, whereas the lower sequence is also referred to as the antisense strand. The upper and lower sequences in the Table can further comprise a chemical modification having Formula I-VII.

TABLE III

| HBV Synthetic siRNA constructs | | | | | |
|---|---|---|---|---|---|
| Target Sequence | Seq ID | Aliases | Sequence | Seq ID | |
| | | HBV (HepBzyme site) as siRNA str1 (sense) | B UGGACUUCUCUCAAUUUUCUA B | 1319 | |
| | | HBV (HepBzyme site) as siRNA str2 | B UAGAAAAUUGAGAGAAGUCCA B | 1320 | |
| | | (antisense) | | | |
| | | HBV18371 site as siRNA str1 (sense) | B UUUUUCACCUCUGCCUAAUCA B | 1321 | |
| | | HBV18371 site as siRNA str2 (antisense) | B UGAUUAGGCAGAGGUGAAAAA B | 1322 | |
| | | HBV16372-18373 site as siRNA str1 (sense) | B CAAGCCUCCAAGCUGUGCCUU B | 1323 | |
| | | HBV16372-18373 site as siRNA str2 | B AAGGCACAGCUUGGAGGCUUG B | 1324 | |
| | | (antisense) | | | |
| | | HBV (HepBzyme site) as siRNA str1 (sense) | B UAGAAAAUUGAGAGAAGUCCA B | 1320 | |
| | | Inverted Control | | | |
| | | HBV (HepBzyme site) as siRNA str1 (sense) | B UGGACUUCUCUCAAUUUUCUA B | 1319 | |
| | | Inverted Control Compliment | | | |
| | | HBV (HepBzyme site) as siRNA | UGGACUUCUCUCAAUUUUCUAUU | 1325 | |
| | | str1 (sense) + 2 | U overhang | | |
| | | HBV (HepBzyme site) as siRNA str2 | UAGAAAAUUGAGAGAAGUCCAUU | 1326 | |
| | | (antisense) + 2 | U overhang | | |
| | | HBV18371 site as siRNA strl (sense) + | UUUUUCACCUCUGCCUAAUCAUU | 1327 | |
| | | 2U overhang | | | |
| | | HBV18371 site as siRNA str2 (antisense) + | UGAUUAGGCAGAGGUGAAAAAUU | 1328 | |
| | | 2U overhang | | | |
| | | HBV16372-18373 site as siRNA | CAAGCCUCCAAGCUGUGCCUUUU | 1329 | |
| | | str1 (sense)-s-2 | U overhang | | |
| | | HBV16372-18373 site as siRNA str 2 | AAGGCACAGCUUGGAGGCUUGUU | 1330 | |
| | | (antisense) + 2 | U overhang | | |
| | | HBV (HepBzyme site) as siRNA | BUGGACUUCUCUCAAUUUUCUAUUB | 1331 | |
| | | str1 (sense) + 2 | U overhang | | |
| | | HBV (HepBzyme site) as siRNA str2 | BUAGAAAAUUGAGAGAAGUCCAUUB | 1332 | |
| | | (antisense) + 2U overhang | | | |
| | | HBV18371 site as siRNA str1 (sense) + | BUUUUUCACCUCUGCCUAAUCAUUB | 1333 | |
| | | 2U overhang | | | |
| | | HBV1 8371 site as siRNA str2 (antisense) + | BUGAUUAGGCAGAGGUGAAAAAUUB | 1334 | |
| | | 2U overhang | | | |
| | | HBV16372-18373 site as siRNA | BCAAGCCUCCAAGCUGUGCCUUUUB | 1335 | |
| | | str1 (sense) + 2U overhang | | | |
| | | HBV1 6372-18373 site as siRNA str2 | BAAGGCACAGCUUGGAGGCUUGUUB | 1336 | |
| | | (antisense) + 2U overhang | | | |
| GAGUCUAGACUCGUGGUGGACUU | 1293 | HBV:248U21 siRNA pos | GUCUAGACUCGUGGUGGACTT | 1337 | |
| AUCCUGCUGCUAUGCCUCAUCUU | 1294 | HBV:414U21 siRNA pos | CCUGCUGCUAUGCCUCAUCTT | 1338 | |
| UUCAAGCCUCCAAGCUGUGCCUU | 1295 | HBV:1867U21 siRNA pos | CAAGCCUCCAAGCUGUGCCTT | 1339 | |
| CAAGCUGUGCCUUGGGUGGCUUU | 1296 | HBV:1877U21 siRNA pos | AGCUGUGCCUUGGGUGGCUTT | 1340 | |
| GAGUCUAGACUCGUGGUGGACUU | 1293 | HBV:228L21 sIRNA neg (248C) | GUCCACCACGAGUCUAGACTT | 1341 | |
| AUCCUGCUGCUAUGCCUCAUCUU | 1294 | HBV:394L21 siRNA neg (414C) | GAUGAGGCAUAGCAGCAGGTT | 1342 | |
| UUCAAGCCUCCAAGCUGUGCCUU | 1295 | HBV:1847L21 siRNA neg (1867C) | GGCACAGCUUGGAGGCUUGTT | 1343 | |
| CAAGCUGUGCCUUGGGUGGCUUU | 1296 | HBV:1857L21 siRNA neg (18770) | AGCCACCCAAGGCACAGCUTT | 1344 | |
| GAGUCUAGACUCGUGGUGGACUU | 1293 | HBV:248U21 siRNA pos inv | CAGGUGGUGCUCAGAUCUGTT | 1345 | |
| AUCCUGCUGCUAUGCCUCAUCUU | 1294 | HBV:414U21 siRNA pos inv | CUAOUCCGUAUCGUCGUCGTT | 1346 | |
| UUCAAGCCUCCAAGCUGUGCCUU | 1295 | HBV:1867U21 siRNA pos inv | CCGUGUCGAACCUCCGAACTT | 1347 | |
| CAAGCUGUGCCUUGGGUGGCUUU | 1296 | HBV:1877U21 siRNApos inv | UCGGUGGGUUCCGUGUCGATT | 1348 | |
| GAGUCUAGACUCGUGGUGGACUU | 1293 | HBV:228L21 siRNA neg (2480) inv | CAGAUCUGAGCACCACCUGTT | 1349 | |
| AUCCUGCUGCUAUGCCUCAUCUU | 1294 | HBV:394L21 siRNA neg (4140) inv | GGACGACGAUACGGAGUAGTT | 1350 | |
| UUCAAGCCUCCAAGCUGUGCCUU | 1295 | HBV:1847L21 siRNA neg (18670) inv | GUUCGGAGGUUCGACACGGTT | 1351 | |
| CAAGCUGUGCCUUGGGUGGCUUU | 1296 | HBV:1857L21 siRNA neg (18770) inv | UCGACACGGAACCCACCGATT | 1352 | |
| AUCCUGCUGCUAUGCCUCAUCUU | 1294 | HBV:414U21 siRNA pos stab3 | c_{S}c_{S}u_{S}G_{S}cuGcuAuGccucA_{S}u_{S}c_{S}T_{S}T | 1353 | |
| AUCCUGCUGCUAUGCCUCAUCUU | 1294 | HBV:414U21 siRNA pos stab4 | B ccuGcuGcuAuGccucAucTTB | 1354 | |
| AUCCUGCUGCUAUGCCUCAUCUU | 1294 | HBV:414U21 siRNApos stab6 | BccuGcuGcuAuGccucAucTTB | 1355 | |
| AUCCUGCUGCUAUGCCUCAUCUU | 1294 | HBV:394L21 siRNA neg (414C) stab2 | G_{S}A_{S}U_{S}G_{S}A_{S}G_{S}G_{S}C_{S}A_{S}U_{S}A_{S}G_{S}C_{S}A_{S}G_{S}C_{S}A_{S}G_{S}G_{S}T_{S}T | 1356 | |
| AUCCUGCUGCUAUGCCUCAUCUU | 1294 | HBV:394L21 siRNA neg (414C) stab5 | GAUGAGGCAUAGCAGCAGGT_{S}T | 1357 | |
| AUCCUGCUGCUAUGCCUCAUCUU | 1294 | HBV:414U21 siRNApos inv stab3 | c_{S}u_{S}A_{S}c_{S}uccGuAucGucG_{S}u_{S}c_{S}c_{S}T_{S}T | 1358 | |
| AUCCUGCUGCUAUGCCUCAUCUU | 1294 | HBV:414U21 siRNApos inv stab4 | BcUAcuccGuAucGucGuccTTB | 1359 | |
| AUCCUGCUGCUAUGCCUCAUCUU | 1294 | HBV:414U21 siRNApos inv stab6 | BcUAcuccGuAucGucGuccTTB | 1360 | |
| AUCCUGCUGCUAUGCCUGAUCUU | 1294 | HBV:394L21 siRNA neg (414C) inv stab2 | G_{S}G_{S}A_{S}C_{S}G_{S}A_{S}C_{S}G_{S}A_{S}U_{S}A_{S}C_{S}G_{S}G_{S}A_{S}G_{S}U_{S}A_{S}G_{S}T_{S}T | 1361 | |
| AUCCUGCUGCUAUGCCUCAUCUU | 1294 | HBV:394L21 siRNA neg (414C) inv stabS | GGAcGAcGAuAcGGAGuAGT_{S}T | 1362 | |
| AUCCUGCUGCUAUGCCUCAUCUU | 1294 | HBV:414U21 siRNApos stab3 | c_{S}c_{S}u_{S}G_{S}cuGcuAuGccucA_{S}u_{S}c_{S}T_{S}T_{S} | 1353 | |
| AUCCUGCUGCUAUGCCUGAUCUU | 1294 | HBV:414U21 siRNApos stab4 | BccuGcuGcuAuGccucAucTTB | 1354 | |
| AU0OUGCUGCUAUGCCUCAUCUU | 1294 | HBV:414U21 siRNApos stab6 | BccuGcuGcuAuGccucAucTTB | 1355 | |
| AUCCUGCUGCUAUGCCUCAUCUU | 1294 | HBV:394L21 siRNA neg (4140) stab2 | G_{S}A_{S}U_{S}G_{S}A_{S}G_{S}G_{S}C_{S}A_{S}U_{S}A_{S}G_{S}C_{S}A_{S}G_{S}C_{S}A_{S}G_{S}G_{S}T_{S}T | 1356 | |
| AUCCUGCUGCUAUGCCUCAUCUU | 1294 | HBV:394L21 siRNA neg (4140) stab5 | GAuGAGGcAuAGcAGcAGGT_{S}T | 1357 | |
| AUCCUGCUGCUAUGCCUCAUCUU | 1294 | HBV:414U21 siRNApos inv stab3 | C_{S}U_{S}A_{S}c_{S}uccGuAucGucG_{S}u_{S}c_{S}c_{S}T_{S}T | 1358 | |
| AUOOUGOUGCUAUGCCUCAUCUU | 1294 | HBV:414U21 siRNApos inv stab4 | B cuAcuccGuAucGucGuccTT B | 1359 | |
| AUCGUGCUGCUAUGCCUCAUCUU | 1294 | HBV:414U21 siRNA P05 inv stab6 | B cuAcuccGuAucGucGuccTT B | 1360 | |
| AUCCUGCUGOUAUGCCUCAUCUU | 1294 | HBV:394L21 siRNA neg (4140) inv stab2 | G_{S}G_{S}A_{S}C_{S}G_{S}A_{S}C_{S}G_{S}A_{S}U_{S}A_{S}C_{S}G_{S}G_{S}A_{S}G_{S}U_{S}A_{S}G_{S}T_{S}T | 1361 | |
| AUCCUGCUGCUAUGCCUCAUCUU | 1294 | HBV:394L21 siRNA neg (4140) inv stab5 | GGAcGAcGAuAcGGAGuAGT_{S}T | 1362 | |
| GGUGGACUUCUCUGAAUUUUCUA | 1297 | HBV:262U21 siRNA | UGGACUUCUCUCAAUUUUCUA | 1363 | |
| GGACUUCUCUGAAUUUUCUAGGG | 1298 | HBV:265U21 siRNA | ACUUCUCUGAAUUUUCUAGGG | 1364 | |
| GAUGUGUCUGCGGCGUUUUAUCA | 1299 | HBV:380U21 siRNA | UGUGUCUGCGGCGUUUUAUCA | 1365 | |
| CAUCCUGCUGCUAUGCCUCAUCU | 1300 | HBV:413U21 siRNA | UCCUGCUGCUAUGCCUCAUCU | 1366 | |
| GGUAUGUUGCCCGUUUGUCGUCU | 1301 | HBV:462U21 siRNA | UAUGUUGCCCGUUUGUCCUCU | 1367 | |
| CGUGUGCACUUCGCUUCACCUCU | 1302 | HBV:1580U21 siRNA | UGUGCACUUCGCUUCACCUCU | 1368 | |
| CACUUCGCUUCACCUCUGCACGU | 1303 | HBV:1586U21 siRNA | CUUCGCUUCACCUCUGCACGU | 1369 | |
| GGAGGCUGUAGGCAUAAAUUGGU | 1304 | HBV:1780U21 siRNA | AGGCUGUAGGGAUAAAUUGGU | 1370 | |
| CUCCAAGCUGUGCCUUGGGUGGC | 1305 | HBV:1874U21 siRNA | CGAAGCUGUGOCUUGGGUGGC | 1371 | |
| CCCUAGAAGAAGAACUCGCUCGC | 1306 | HBV:2369U21 siRNA | CUAGAAGAAGAACUOOCUCGO | 1372 | |
| GAAGAAGAACUCCCUCGCCUCGC | 1307 | HBV:2374U21 siRNA | AGAAGAACUCCCUCG0 | CUCGC | 1373 |
| GGUGGACUUCUCUCAAUUUUCUA | 1297 | HBV:280L21 siRNA (262C) | GAAAAUUGAGAGAAGUCCACC | 1374 | |
| GGACUUCUCUCAAUUUUCUAGGG | 1298 | HBV:283L21 siRNA (265C) | CUAGAAAAUUGAGAGAAGUCC | 1375 | |
| GAUGUGUCUGCGGCGUUUUAUCA | 1299 | HBV:398L21 siRNA (380C) | AUAAAACGCGGGAGACACAUC | 1376 | |
| GAUCCUGCUGCUAUGCCUCAUCU | 1300 | HBV:431L21 siRNA (413C) | AUGAGGCAUAGCAGCAGGAUG | 1377 | |
| GGUAUGUUGCCGGUUUGUCCUCU | 1301 | HBV:480L21 siRNA (462C) | AGGACAAACGGGCAACAUACC | 1378 | |
| CGUGUGCACUUCGCUUCACCUCU | 1302 | HBV:1598L21 siRNA (1580C) | AGGUGAAGCGAAGUGCACACG | 1379 | |
| CACUUCGCUUCACCUCUGCACGU | 1303 | HBV:1604L21 siRNA (1586C) | GUGCAGAGGUGAAGCGAAGUG | 1380 | |
| GGAGGCUGUAGGCAUAAAUUGGU | 1304 | HBV:1798L21 siRNA (1780C) | CAAUUUAUGCCUACAGCCUC0 | 1381 | |
| CUCCAAGCUGUGCCUUGGGUGGC | 1305 | HBV:1892L21 siRNA (1874C) | ACCCAAGGGACAGCUUGGAG | 1382 | |
| CCCUAGAAGAAGAACUCCGUCGC | 1306 | HBV:2387L21 siRNA (2369C) | GAGGGAGUUCUUCUUCUAGGG | 1383 | |
| GAAGAAGAACUCCCUCGCCUCGC | 1307 | HBV:2392L21 siRNA (2374C) | GAGGCGAGGGAGUUCUUOUUC | 1384 | |
| AUOUUUUAACUCUCUUGAGGUGG | 1308 | HBV:260U21 siRNA inv | CUUUUAACUCUOUUGAGGUGG | 1385 | |
| GGGAUCUUUUAACUCUCUUCAGG | 1309 | HBV:263U21 siRNA inv | GAUCUUUUAACUOUOUUGAGG | 1386 | |
| AOUAUUUUGCGGCGUOUGUGUAG | 1310 | HBV:378U21 siRNA inv | UAUUUUGCGGCGUCUGUGUAG | 1387 | |
| UCUACUCCGUAUCGUCGUCGUAC | 1311 | HBV:411U21 siRNA inv | UACUCCGUAUCGUCGUCCUAC | 1388 | |
| UCUCCUGUUUGCCCGUUGUAUGG | 1312 | HBV:460U21 siRNA inv | UCGUGUUUGCCCGUUGUAUGG | 1389 | |
| UCUCGACUUCGCUUCACGUGUGC | 1313 | HBV:1578U21 siRNA inv | UCGACUUCGCUUCACGUGUGC | 1390 | |
| UGCACGUCUCCACUUCGCUUCAC | 1314 | HBV:1584U21 siRNA inv | GACGUCUCGACUUCGCUUCAC | 1391 | |
| UGGUUAAAUACGGAUGUCGGAGG | 1315 | HBV:1778U21 siRNA inv | GUUAAAUACGGAUGUCGGAGG | 1392 | |
| CGGUGGGUUCCGUGUCGAACCUC | 1316 | HBV:1872U21 siRNA inv | GUGGGUUCCGUGUCGAACCUC | 1393 | |
| CGCUCCCUGAAGAAGAAGAUCCC | 1317 | HBV:2367U21 siRNA inv | CUCCCUGAAGAAGAAGAUCCC | 1394 | |
| CGCUCCGCUCCCUGAAGAAGAAG | 1318 | HBV:2372U21 siRNA inv | CUCCGCUCCCUGAAGAAGAAG | 1395 | |
| AUCUUUUAACUCUCUUGAGGUGG | 1308 | bogus HBV:282L21 siRNA (260C) inv | ACCUGAAGAGAGUUAAAAGAU | 1396 | |
| GGGAUCUUUUAACUCUCUUCAGG | 1309 | HBV:285L21 siRNA (263C) inv | UGAAGAGAGUUAAAAGAUCCG | 1397 | |
| ACUAUUUUGCGGCGUCUGUGUAG | 1310 | HBV:400L21 siRNA (378C) inv | AGAGAGACGCCGGAAAAUAGU | 1398 | |
| UCUACUCCGUAUCGUCGUCCUAC | 1311 | HBV:433L21 siRNA (411C) inv | AGGACGACGAUACGGAGUAGA | 1399 | |
| UCUCCUGUUUGCCCGUUGUAUGG | 1312 | HBV:482L21 siRNA (460C) inv | AUAGAACGGGGAAAGAGGAGA | 1400 | |
| UCUCCACUUCGCUUGACGUGUGC | 1313 | HBV:1600L21 siRNA (1578C) inv | AGACGUGAAGCGAAGUGGAGA | 1401 | |
| UGCACGUCUCCACUUCGCUUCAC | 1314 | HBV:1606L21 siRNA (1584C) inv | GAAGCGAAGUGGAGACGUGCA | 1402 | |
| UGGUUAAAUACGGAUGUCGGAGG | 1315 | HBV:1800L21 siRNA (1778C) inv | UCCGACAUCCGUAUUUAACCA | 1403 | |
| CGGUGGGUUCCGUGUCGAACCUC | 1316 | HBV:1894L21 siRNA (1872C) inv | GGUUCGACACGGAACCCACCG | 1404 | |
| CGCUCCCUCAAGAAGAAGAUCCC | 1317 | HBV:2389L21 siRNA (2367C) inv | GAUCUUCUUCUUGAGGGAGCG | 1405 | |
| CGCUCCGCUCCCUCAAGAAGAAG | 1318 | HBV:2394L21 siRNA (2372C) inv | UCUUCUUGAGGGAGCGGAGCG | 1406 | |
| GGUGGACUUCUCUCAAUUUUCUA | 1297 | HBV:262U21 siRNA stab4 | BuGGAcuucucucAAuuuucuAB | 1407 | |
| GGACUUCUCUCAAUUUUCUAGGG | 1298 | HBV:265U21 siRNA stab4 | BAcuucucucAAuuuucuAGGGB | 1408 | |
| GAUGUGUCUGCGGCGUUUUAUCA | 1299 | HBV:380U21 siRNA stab4 | BuGuGucuGcGGcGuuuuAucAB | 1409 | |
| CAUCCUGCUGCUAUGCCUCAUCU | 1300 | HBV:413U21 siRNA stab4 | BuccuGcuGcuAuGccucAucuB | 1410 | |
| GGUAUGUUGCCCGUUUGUCCUCU | 1301 | HBV:462U21 siRNAstab4 | BuAuGuuGcccGuuuGuccucuB | 1411 | |
| CGUGUGCACUUCGCUUCACCUCU | 1302 | HBV:1586U21 siRNA stab4 | BuGuGcAcuucGcuucAccucuB | 1412 | |
| CACUUCGCUUCACCUCUGCACGU | 1303 | HBV:1586U21 siRNA stab4 | BcuucGcuucAccucuGcAcGuB | 1413 | |
| GGAGGCUGUAGGCAUAAAUUGGU | 1304 | HBV:1780U21 siRNA stab4 | BAGGcuGuAGGcAuAAAuuGGuB | 1414 | |
| CUCCAAGCUGUGCCUUGGGUGGC | 1305 | HBV:1874U21 siRNAstab4 | BccAAGcuGuGccuuGGGuGGcB | 1415 | |
| CCCUAGAAGAAGAACUCCCUCGC | 1306 | HBV:2369U21 siRNA stab4 | BcuAGAAGAAGAAcucccucGcB | 1416 | |
| GAAGAAGAACUCCCUCGCCUCGC | 1307 | HBV:2374U21 siRNAstab4B | AGAAGAAcucccucGccucGcB | 1417 | |
| GGUGGACUUCUCUCAAUUUUCUA | 1297 | HBV:280L21 siRNA (262C) stab5 | GAAAAuuGAGAGAAGuccAT_{S}T | 1418 | |
| GGACUUCUCUCAAUUUUCUAGGG | 1298 | HBV:283L21 siRNA (265C) stab5 | cuAGAAAAGuuGAGAGAAGuT_{S}T | 1419 | |
| GAUGUGUCUGCGGCGUUUUAUGA | 1299 | HBV:398L21 siRNA (380C) stab5 | AuAAAAcGccGcAGAcAcAT_{S}T | 1420 | |
| GAUCCUGCUGCUAUGCCUCAUCU | 1300 | HBV:431L21 siRNA (413C) stab5 | AuGAGGcAUAGcAGcAGGAT_{S}T | 1421 | |
| GGUAUGUUGCCCGUUUGUCCUCU | 1301 | HBV:480L21 siRNA (462C) stab5 | AGGAcAAAcGGGcAACAUAT_{S}T | 1422 | |
| CGUGUGCACUUCGCUUCACCUCU | 1302 | HBV:1598L21 siRNA (158CC) stab5 | AGGuGAAGcGAAGuGcAcAT_{S}T | 1423 | |
| CACUUCGCUUGACCUCUGCACGU | 1303 | HBV:1604L21 siRNA (1586C) stab5 | GuGcAGAGGuGAAGcGAAGT_{S}T | 1424 | |
| GGAGGCUGUAGGGAUAAAUUGGU | 1304 | HBV:1798L21 siRNA (178CC) stab5 | cAAuuuAuGccuAcAGccuT_{S}T | 1425 | |
| CUCCAAGCUGUGCGUUGGGUGGC | 1305 | HBV:1892L21 siRNA (1874C) stab5 | cAcccAAGGcAcAGCuuGGT_{S}T | 1426 | |
| CCCUAGAAGAAGAACUCCCUCGC | 1306 | HBV:2387L21 siRNA (2369C) stab5 | GAGGGAGuucuucuucuAGT_{S}T | 1427 | |
| GAAGAAGAACUCCCUCGCCUCGC | 1307 | HBV:2392L21 siRNA (2374C) stab5 | GAGGcGAGGGAGuucuucuT_{S}T | 1428 | |
| GGUGGACUUCUCUGAAUUUUCUA | 1297 | HBV:262U21 siRNA inv stab4 | BAucuuuuAAcucucuucAGGuB | 1429 | |
| GGACUUCUCUGAAUUUUCUAGGG | 1298 | HBV:265U21 siRNA inv stab4 | BGGGAucuuuuAAcucucuucAB | 1430 | |
| GAUGUGUCUGCGGCGUUUUAUGA | 1299 | HBV:380U21 siRNA inv stab4 | BAcuAuuuuGcGGcGucucGuGuB | 1431 | |
| CAUCCUGCUGCUAUGCCUGAUCU | 1300 | HBV:413U21 siRNA inv stab4 | BucuAcuccGuAucGucGuccuB | 1432 | |
| GGUAUGUUGCCGGUUUGUCCUCU | 1301 | HBV:462U21 siRNA inv stab4 | BucuccuGuuuGcccGuuGuAuB | 1433 | |
| CGUGUGGACUUCGCUUCACCUCU | 1302 | HBV:1580U21 siRNA inv stab4 | BucuccAcuucGcuucAcGuGuB | 1434 | |
| GACUUCGCUUGACCUCUGGACGU | 1303 | HBV:1586U21 siRNA inv stab4 | BuGcAcGucuccAcuucGcuucB | 1435 | |
| GGAGGCUGUAGGGAUAAAUUGGU | 1304 | HBV:1780U21 siRNA inv stab4 | BuGGuuAAAuAcGGAuGucGGAB | 1436 | |
| CUCGAAGCUGUGCCUUGGGUGGC | 1305 | HBV:1874U21 siRNA inv stab4 | BcGGuGGGuuccGuGucGAAccB | 1437 | |
| CCCUAGAAGAAGAACUCCCUCGC | 1306 | HBV:2369U21 siRNA inv stab4 | BcGcucccucAAGAAGAAGAucB | 1438 | |
| GAAGAAGAACUCCCUCGCCUCGC | 1307 | HBV:2374U21 siRNA inv stab4 | BcGcuccGcuccucAAGAAGAB | 1439 | |
| GGUGGACUUCUCUCAAUUUUCUA | 1297 | HBV:280L21 siRNA (262C) inv stab5 | AccuGAAGAGAGuuAAAAGT_{S}T | 1440 | |
| GGACUUCUCUCAAUUUUCUAGGG | 1298 | HBV:283L21 siRNA (265C) inv stab5 | uGAAGAGAGuuAAAAGAuCT_{S}T | 1441 | |
| GAUGUGUCUGCGGCGUUUUAUCA | 1299 | HBV:398L21 siRNA (380C) inv stab5 | AcAcAGAcGCccGcAAAAuAT_{S}T | 1442 | |
| CAUCCUGCUGCUAUGCGUCAUCU | 1300 | HBV:431L21 siRNA (413C) inv stab5 | AGGAcGAcGAuAcGGAGuAT_{S}T | 1443 | |
| GGUAUGUUGCCCGUUUGUCCUCU | 1301 | HBV:480L21 siRNA (462C) inv stab5 | AuAcGAAcGGGcAAAcAGGAT_{S}T | 1444 | |
| CGUGUGCACUUCGCUUCACCUCU | 1302 | HBV:1598L21 siRNA (158CC) inv stab5 | AcAcGuGAAGcGAAGuGGAT_{S}T | 1445 | |
| CACUUCGCUUCACCUCUGGACGU | 1303 | HBV:1604L21 siRNA (1586C) inv stab5 | GAAGcGAAGuGGAGAcGuGT_{S}T | 1446 | |
| GGAGGCUGUAGGCAUAAAUUGGU | 1304 | HBV:1798L21 siRNA (1780C) inv stab5 | uccGAcAuccGuAuuuAAcT_{S}T | 1447 | |
| CUCCAAGCUGUGCCUUGGGUGGC | 1305 | HBV:1892L21 siRNA (1874C) inv stab5 | GGuucGAcAcGGAAcccAcT_{S}T | 1448 | |
| CCCUAGAAGAAGAACUCCCUCGC | 1306 | HBV:2387L21 siRNA (2369C) inv stab5 | GAucuucuucuuGAGGGAGT_{S}T | 1449 | |
| GAAGAAGAACUCCCUCGCCUCGC | 1307 | HBV:2392L21 siRNA (2374C) inv stab5 | ucuucuuGAGGGAGcGGAGT_{S}T | 1450 | |
| GGUGGACUUCUCUCAAUUUUCUA | 1297 | HBV:262U21 siRNA inv | AUCUUUUAACUCUCUUCAGGU | 1451 | |
| GGACUUCUCUCAAUUUUCUAGGG | 1298 | HBV:265U21 siRNA inv | GGGAUCUUUUAACUCUCUUCA | 1452 | |
| GAUGUGUCUGCGGCGUUUUAUCA | 1299 | HBV:380U21 siRNA inv | ACUAUUUUGCGGCGUCUGUGU | 1453 | |
| CAUCCUGCUGCUAUGCGUGAUCU | 1300 | HBV:413U21 siRNA inv | UCUACUCCGUAUCGUCGUCCU | 1454 | |
| GGUAUGUUGCGCGUUUGUCCUCU | 1301 | HBV:462U21 siRNA inv | UCUCCUGUUUGCCCGUUGUAU | 1455 | |
| CGUGUGCACUUCGCUUGACCUCU | 1302 | HBV:1580U21 siRNA inv | UCUCCACUUCGCUUGACGUGU | 1456 | |
| GACUUCGCUUCACCUCUGCACGU | 1303 | HBV:1586U21 siRNA inv | UGGACGUCUCGACUUCGCUUC | 1457 | |
| GGAGGCUGUAGGGAUAAAUUGGU | 1304 | HBV:1780U21 siRNA inv | UGGUUAAAUACGGAUGUCGGA | 1458 | |
| CUCCAAGCUGUGCCUUGGGUGGC | 1305 | HBV:1874U21 siRNA inv | CGGUGGGUUCCGUGUCGAACG | 1459 | |
| CGCUAGAAGAAGAACUCCCUCGC | 1306 | HBV:2369U21 siRNA inv | CGCUCCCUGAAGAAGAAGAUC | 1460 | |
| GAAGAAGAACUCGCUCGCGUCGC | 1307 | HBV:2374U21 siRNA inv | CGCUCCGCUCCCUGAAGAAGA | 1461 | |
| GGUGGACUUCUCUCAAUUUUCUA | 1297 | HBV:280L21 siRNA (262C) inv | CGACCUGAAGAGAGUUAAAAG | 1462 | |
| GGACUUCUCUGAAUUUUCUAGGG | 1298 | HBV:283L21 siRNA (265C) inv | CCUGAAGAGAGUUAAAAGAUC | 1463 | |
| GAUGUGUCUGCGGCGUUUUAUGA | 1299 | HBV:398L21 siRNA (380C) inv | CUACAGAGACGCCGGAAAAUA | 1464 | |
| GAUCCUGCUGCUAUGCCUGAUCU | 1300 | HBV:431L21 siRNA (413C) inv | GUAGGACGACGAUACGGAGUA | 1465 | |
| GGUAUGUUGCCCGUUUGUCGUCU | 1301 | HBV:480L21 siRNA (462C) inv | CGAUAGAACGGGGAAAGAGGA | 1466 | |
| CGUGUGGACUUCGCUUGACGUCU | 1302 | HBV:1598L21 siRNA (1580C) inv | GGAGACGUGAAGCGAAGUGGA | 1467 | |
| GACUUCGCUUGACCUCUGCACGU | 1303 | HBV:1604L21 siRNA (1586C) inv | GUGAAGCGAAGUGGAGACGUG | 1468 | |
| GGAGGCUGUAGGGAUAAAUUGGU | 1304 | HBV:1798L21 siRNA (1780C) inv | CCUCCGAGAUCCGUAUUUAAC | 1469 | |
| CUCGAAGCUGUGCGUUGGGUGGC | 1305 | HBV:1892L21 siRNA (1874C) inv | GAGGUUCGAGACGGAACCCAC | 1470 | |
| CCCUAGAAGAAGAACUCCCUCGC | 1306 | HBV:2387L21 siRNA (2369C) inv | GGGAUCUUCUUCUUGAGGGAG | 1471 | |
| GAAGAAGAACUCCCUCGCCUCGC | 1307 | HBV:2392L21 siRNA (2374C) inv | CUUCUUCUUGAGGGAGCGGAG | 1472 | |
| GGUGGACUUCUCUGAAUUUUCUA | 1297 | HBV:262U21 siRNA stab7 | BuGGAcuucucucAAuuuucTTB | 1473 | |
| GAUGUGUCUGCGGCGUUUUAUCA | 1299 | HBV:380U21 siRNA stab7 | BuGuGucuGcGGcGuuuuAuTTB | 1474 | |
| CAUCCUGCUGCUAUGCCUCAUCU | 1300 | HBV:413U21 siRNA stab7 | BuccuGcuGcuAuGccucAuTTB | 1475 | |
| AUCCUGCUGCUAUGCCUCAUCUU | 1294 | HBV:414U21 siRNA stab7 | BccuGcuGcuAuGccucAucTTB | 1476 | |
| GGUAUGUUGCCCGUUUGUCCUCU | 1301 | HBV:462U21 siRNAstab7 | BuAuGuuGcccGuuuGuccuTTB | 1477 | |
| CGUGUGCACUUCGCUUCACCUCU | 1302 | HBV:1580U21 siRNA stab7 | BuGuGcAcuucGcuucAccuTTB | 1478 | |
| CACUUCGCUUCACCUCUGCACGU | 1303 | HBV:1580U21 siRNA stab7 | BcuccuGcuucAccucuGcAcTTB | 1479 | |
| GGAGGCUGUAGGCAUAAAUUGGU | 1304 | HBV:1780U21 siRNA stab7 | BAGGcuGuAGGcAuAAAuuGTTB | 1480 | |
| GGUGGACUUCUCUCAAUUUUCUA | 1297 | HBV:280L21 siRNA (262C) | stab8 | gaaaauugagagaaguccaT_{S}T | 1481 |
| GAUGUGUCUGCGGCGUUUUAUCA | 1299 | HBV:398L21 siRNA (380C) | stab8 | auaaaacgccgcagacacaT_{S}T | 1482 |
| GAUCCUGCUGCUAUGCCUCAUCU | 1300 | HBV:431L21 siRNA (413C) | stab8 | augaggcauagcagcaggaT_{S}T | 1483 |
| AUCCUGCUGCUAUGCCUCAUCUU | 1294 | HBV:394L21 siRNA (414C) | stab8 | gaugaggcauagcagcaggT_{S}T | 1484 |
| GGUAUGUUGCCCGUUUGUCCUCU | 1301 | HBV:480L21 siRNA (462C) | stab8 | aggacaaacgggcaacauaT_{S}T | 1485 |
| CGUGUGCACUUCGCUUCACCUCU | 1302 | HBV:1598L21 siRNA (1580C) | stab8 | aggugaagcgaagugcacaT_{S}T | 1486 |
| CACUUCGCUUCACCUCUGCACGU | 1303 | HBV:1604L21 siRNA (1586C) | stab8 | gugcagaggugaagcgaagT_{S}T | 1487 |
| GGAGGCUGUAGGCAUAAAUUGGU | 1304 | HBV:1798L21 siRNA (1780C) | stab8 | caauuuaugccuacagccuT_{S}T | 1488 |
| GGUGGACUUCUCUCAAUUUUCUA | 1297 | HBV:262U21 siRNA inv stab7 | BAucuuuuAAcucucuucAGTTB | 1489 | |
| GAUGUGUCUGCGGCGUUUUAUCA | 1299 | HBV:380U21 siRNA invstab7 | BAcuAuuuuGcGGcGucuGuTTB | 1490 | |
| CAUCCUGCUGCUAUGCCUCAUCU | 1300 | HBV:413U21 siRNA inv stab7 | BucuAcuccGuAucGucGucTTB | 1491 | |
| AUCCUGCUGCUAUGCCUCAUCUU | 1294 | HBV:414U21 siRNA inv stab7 | BcuAcuccGuAucGucGuccTTB | 1492 | |
| GGUAUGUUGCCCGUUUGUCCUCU | 1301 | HBV:462U21 siRNA inv stab7 | BucuccuGuuuGcccGuuGuTTB | 1493 | |
| CGUGUGCACUUCGCUUCACCUCU | 1302 | HBV:1580U21 siRNA inv stab7 | BucuccAcuucGcuucAcGuTTB | 1494 | |
| CACUUCGCUUCACCUCUGCACGU | 1303 | HBV:1586U21 siRNA inv stab7 | BuGcAcGucuccAcuucGcuTTB | 1495 | |
| GGAGGCUGUAGGCAUAAAUUGGU | 1304 | HBV:1780U21 siRNA inv stab7 | BuGGuuAAAuAcGGAuGucGTTB | 1496 | |
| GGUGGACUUCUCUGAAUUUUCUA | 1297 | HBV:280L21 siRNA (262C) inv stab8 | ccaccugaagagaguuaaaT_{S}T | 1497 | |
| GAUGUGUCUGCGGCGUUUUAUCA | 1299 | HBV:398L21 siRNA (380C) inv stab8 | cuacacagacgccgcaaaaT_{S}T | 1498 | |
| CAUCCUGCUGCUAUGCCUCAUCU | 1300 | HBV:431L21 siRNA (413C) inv stab8 | guaggacgacgauacggagT_{S}T | 1499 | |
| AUCGUGCUGCUAUGCCUCAUCUU | 1294 | HBV:394L21 siRNA (414C) inv stab8 | ggacgacgauacggaguagT_{S}T | 1500 | |
| GGUAUGUUGCCCGUUUGUCCUCU | 1301 | HBV:480L21 siRNA (462C) inv stab8 | ccauacaacgggcaaacagT_{S}T | 1501 | |
| CGUGUGCACUUCGCUUGACGUCU | 1302 | HBV:1598L21 siRNA (1580C) inv stab8 | gcacacgugaagcgaagugT_{S}T | 1502 | |
| GACUUCGCUUGACCUCUGCACGU | 1303 | HBV:1604L21 siRNA (1586C) inv stab8 | gugaagcgaaguggagacgT_{S}T | 1503 | |
| GGAGGCUGUAGGCAUAAAUUGGU | 1304 | HBV:1798L21 siRNA (1780C) inv stab8 | ccuccgacauccguauuuaT_{S}T | 1504 | |

A = Adenosine
G = Guanosine
C = Cytidine
U = Uridine
T = Thymidine
A = 2′-deoxy Adenosine
G = 2′-deoxy Guanosine
U = 2′-deoxy-2′-fluoro uridine
C = 2′-deoxy-2′-fluoro cytidine
a = 2′-O-methyl uridine
C = 2′-O-methyl cytidine
B = inverted deoxy abasic ribose
L = glyceryl moiety
S = phosphorothioate internucleotide linkage

TABLE IV
A. 2.5 μmol Synthesis Cycle ABI 394 Instrument

| Reagent | Equivalents | Amount | Wait Time* DNA | Wait Time* 2′-0-methyl | Wait Time*RNA |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| Reagent | Equivalents | Amount | Wait Time* DNA | Wait Time* 2′-O-methyl | Wait Time*RNA |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents:DNA/ | Amount: DNA/2′-O- | | Wait Time* 2′-O- | |
|---|---|---|---|---|---|
| Reagent | 2′-O-methyl/Ribo | methyl/Ribo | Wait Time* DNA | methyl | Wait Time* Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

Wait time does not include contact time during delivery.
Tandem synthesis utilizes double coupling of linker molecule

What we claim is:

1. A short interfering nucleic acid (siNA) molecule that down-regulates expression of a HBV gene by RNA interference.

2. A short interfering nucleic acid (siNA) molecule that inhibits HBV replication.

3. The siNA molecule of claim 1, wherein the HBV gene encodes sequence comprising Genbank Accession number AB073834.

4. The siNA molecule of claim 1, wherein said siNA molecule is adapted for use to treat HBV infection.

5. The siNA molecule of claim 1, wherein said siNA molecule comprises a sense region and an antisense region and wherein said antisense region comprises sequence complementary to an RNA sequence encoding HBV and the sense region comprises sequence complementary to the antisense region.

6. The siNA molecule of claim 5, wherein said siNA molecule is assembled from two nucleic acid fragments wherein one fragment comprises the sense region and the second fragment comprises the antisense region of said siNA molecule.

7. The siNA molecule of claim 6, wherein said sense region and said antisense region comprise separate oligonucleotides.

8. The siNA molecule of claim 6, wherein said sense region and said antisense region are covalently connected via a linker molecule.

9. The siNA molecule of claim 8, wherein said linker molecule is a polynucleotide linker.

10. The siNA molecule of claim 8, wherein said linker molecule is a non-nucleotide linker.

11. The siNA molecule of claim 1, wherein the siNA molecule comprises sequence having any of SEQ ID NOs.: 1-1524.

12. The siNA molecule of claim 5, wherein said sense region comprises a 3′-terminal overhang and said antisense region comprises a 3′-terminal overhang.

13. The siNA molecule of claim 12, wherein said 3′-terminal overhangs each comprise about 2 nucleotides.

14. The siNA molecule of claim 12, wherein said antisense region 3′-terminal overhang is complementary to RNA encoding HBV.

15. The siNA molecule of claim 5, wherein said sense region comprises one or more 2′-O-methyl pyrimidine nucleotides and one or more 2′-deoxy purine nucleotides.

16. The siNA molecule of claim 5, wherein any pyrimidine nucleotides present in said sense region comprise 2′-deoxy-2′-fluoro pyrimidine nucleotides and wherein any purine nucleotides present in said sense region comprise 2′-deoxy purine nucleotides.

17. The siNA molecule of claim 16, wherein any nucleotides comprising a 3′-terminal nucleotide overhang that are present in said sense region are 2′-deoxy nucleotides.

18. The siNA molecule of claim 5, wherein said sense region comprises a 3′-end, a 5′-end, and a terminal cap moiety at 3′-end, the 5′-end, or both of the 5′- and 3′-ends of said sense region.

19. The siNA molecule of claim 18, wherein said terminal cap moiety is an inverted deoxy abasic moiety.

20. The siNA molecule of claim 5, wherein said antisense region comprises one or more 2′-deoxy-2′-fluoro pyrimidine nucleotides and one or more 2′-O-methyl purine nucleotides.

21. The siNA molecule of claim 5, wherein any pyrimidine nucleotides present in said antisense region comprise 2′-deoxy-2′-fluoro pyrimidine nucleotides and wherein any purine nucleotides present in said antisense region comprise 2′-O-methyl purine nucleotides.

22. The siNA molecule of claim 21, wherein any nucleotides comprising a 3′-terminal nucleotide overhang that are present in said antisense region are 2′-deoxy nucleotides.

23. The siNA molecule of claim 5, wherein said antisense region comprises a phosphorothioate internucleotide linkage at the 3′-end of said antisense region.

24. The siNA molecule of claim 5, wherein said antisense region comprises a glyceryl modification at the 3′-end of said antisense region.

25. The siNA molecule of claim 12, wherein said 3′-terminal nucleotide overhangs comprise deoxyribonucleotides.

26. An expression vector comprising a nucleic acid sequence encoding at least one siNA molecule of claim 1 in a manner that allows expression of the nucleic acid molecule.

27. A mammalian cell comprising an expression vector of claim 26.

28. The mammalian cell of claim 27, wherein said mammalian cell is a human cell.

29. The expression vector of claim 26, wherein said at least one siNA molecule comprises a sense region and an antisense region and wherein said antisense region comprises sequence complementary to an RNA sequence encoding HBV and the sense region comprises sequence complementary to the antisense region.

30. The expression vector of claim 26, wherein said at least one siNA molecule comprises two distinct strands having complementary sense and antisense regions.

31. The expression vector of claim 26, wherein said siNA molecule comprises a single strand having complementary sense and antisense regions.
